(19) United States
(12) Patent Application Publication
(10) Pub. No.: US 2008/0026996 A1
(43) Pub. Date: Jan. 31, 2008

(54) Selective Vpac2 Receptor Reptide Agonists

(75) Inventors: Bengt Krister Bokvist, Hamburg (DE); Jesper Lindgren Gromada, Hamburg (DE); Robert Chadwick Cummins, Brownsburg, IN (US); Wolfgang Glaesner, Indianapolis, IN (US); John Philip Mayer, Indianapolis, IN (US); Lianshan Zhang, Carmel, IN (US); Jorge Alsina-Fernandez, Indianapolis, IN (US)

Correspondence Address: ELI LILLY & COMPANY, PATENT DIVISION, P.O. BOX 6288, INDIANAPOLIS, IN, 46206-6288, US

(73) Assignee: ELI LILLY AND COMPANY, Indianapolis, IN (US)

(21) Appl. No.: 11/569,258

(22) PCT Filed: May 19, 2005
(86) PCT No.: PCT/US05/17435

§ 371 Date: Nov. 17, 2006

Related U.S. Application Data
(60) Provisional application No. 60/573,739, filed on May 21, 2004.
(60) Provisional application No. 60/627,880, filed on Nov. 12, 2004.
(60) Provisional application No. 60/656,601, filed on Feb. 25, 2005.

(51) Int. Cl. C07K 14/475; A61K 38/18; A61P 3/10

(52) U.S. Cl. 514/12; 530/324

(57) ABSTRACT

The present invention encompasses peptides that selectively activate the VPAC2 receptor and are useful in the treatment of diabetes.

The present invention is in the field of medicine. More particularly, this invention relates to selective VPAC2 receptor peptide agonists.

Type 2 diabetes, or non-insulin dependent diabetes mellitus (NIDDM), is the most common form of diabetes, affecting 90% of people with diabetes. With NIDDM, patients have impaired β-cell function resulting in insufficient insulin production and/or decreased insulin sensitivity. If NIDDM is not controlled, excess glucose accumulates in the blood, resulting in hyperglycemia. Over time, more serious complications may arise including renal dysfunction, cardiovascular problems, visual loss, lower limb ulceration, neuropathy, and ischemia. Treatments for NIDDM include improving diet, exercise, and weight control as well as using a variety of oral medications. Individuals with NIDDM can initially control their blood glucose levels by taking such oral medications. These medications, however, do not slow the progressive loss of β-cell function that occurs in type 2 diabetes patients and, thus, are not sufficient to control blood glucose levels in the later stages of the disease. Also, treatment with currently available medications exposes NIDDM patients to potential side effects such as hypoglycemia, gastrointestinal problems, fluid retention, oedema, and/or weight gain.

Compounds, such as peptides that are selective for a particular G-protein coupled receptor known as the VPAC2 receptor, were initially identified by modifying vasoactive intestinal peptide (VIP) and/or pituitary adenylate cyclase-activating polypeptide (PACAP). (See, for example, Xia et al., J Pharmacol Exp Ther., 281:629-633 (1997); Tsutsumi et al., Diabetes, 51:1453-1460 (2002), WO 01/23420, WO 2004/006839.) Many of these peptides are not, however, suitable for commercial candidates as a result of stability issues associated with the polypeptides in formulation, as well as issues with the short half-life of these polypeptides.

PACAP belongs to the secretin/glucagon/vasoactive intestinal peptide (VIP) family of peptides and works through three G-protein-coupled receptors that exert their action through the cAMP-mediated and other Ca²⁺-mediated signal transduction pathways. These receptors are known as the PACAP-preferring type 1 (PAC1) receptor (Isobe, et al., Regul. Pept., 110:213-217 (2003); Ogi, et al., Biochem. Biophys. Res. Commun., 196:1511-1521 (1993)) and the two VIP-shared type 2 receptors (VPAC1 and VPAC2) (Sherwood et al., Endocr. Rev., 21:619-670 (2000); Hammar et al., Pharmacol Rev, 50:265-270 (1998); Couvineau, et al., J. Biol. Chem., 278:24759-24766 (2003); Sreedharan, et al., Biochem. Biophys. Res. Commun., 193:546-553 (1993); Lutz, et al., FEBS Lett., 458: 197-203 (1999); Adamou, et al., Biochem. Biophys. Res. Commun., 209: 385-392 (1995)).

PACAP has comparable activities towards all three receptors, while VIP selectively activates the two VPAC receptors (Tsutsumi 2002). Both VIP (Eriksson et al., Peptides, 10: 481-484 (1989)) and PACAP (Filipsson et al., JCEM, 82:3093-3098 (1997)) have been shown to not only stimulate insulin secretion in man when given intravenously but also increase glucagon secretion and hepatic glucose output. As a consequence, PACAP or VIP stimulation generally does not result in a net improvement of glycemia. Activation of multiple receptors by PACAP or VIP also has broad physiological effects on nervous, endocrine, cardiovascular, reproductive, muscular, and immune systems (Gozes et al., Curr. Med. Chem., 6:1019-1034 (1999)). Furthermore, it appears that VIP-induced watery diarrhoea in rats is mediated by only one of the VPAC receptors, VPAC1 (Ito et al., Peptides, 22:1139-1151 (2001); Tsutsumi 2002). In addition, the VPAC1 and PAC1 receptors are expressed on α-cells and hepatocytes and, thus, are most likely involved in the effects on hepatic glucose output.

Recent studies have shown that peptides selective for the VPAC2 receptor are able to stimulate insulin secretion from the pancreas without gastrointestinal (GI) side effects and without enhancing glucagon release and hepatic glucose output (Tsutsumi 2002). Many of the VPAC2 receptor peptide agonists reported to date have, however, less than desirable potency, selectivity, and stability profiles, which could impede their clinical viability.

There is, therefore, a need for new therapies, which overcome the problems associated with current medications for NIDDM. The present invention seeks to provide improved compounds that are selective for the VPAC2 receptor and which induce insulin secretion from the pancreas only in the presence of high blood glucose levels. The compounds of the present invention are peptides, which are believed to also improve beta cell function. These peptides can, therefore, have the physiological effect of inducing insulin secretion without GI side effects or a corresponding increase in hepatic glucose output and also generally have enhanced selectivity, potency, and/or in vivo stability of the peptide compared to known VPAC2 receptor peptide agonists. The compounds of the present invention include selective VPAC2 receptor peptide agonists.

According to a first aspect of the present invention, there is provided a VPAC2 receptor peptide agonist comprising a sequence of the formula:

Formula 10 (SEQ ID NO: 18)
Xaa₁-Xaa₂-Xaa₃-Xaa₄-Xaa₅-Xaa₆-Thr-Xaa₈-Xaa₉-Xaa₁₀-
Thr-Xaa₁₂-Xaa₁₃-Xaa₁₄-Xaa₁₅-Xaa₁₆-Xaa₁₇-Ala-Xaa₁₉-
Xaa₂₀-Xaa₂₁-Xaa₂₂-Xaa₂₃-Xaa₂₄-Xaa₂₅-Xaa₂₆-Xaa₂₇-
Xaa₂₈-Xaa₂₉-Xaa₃₀-Xaa₃₁-Xaa₃₂-Xaa₃₃-Xaa₃₄-Xaa₃₅-
Xaa₃₆-Xaa₃₇-Xaa₃₈-Xaa₃₉-Xaa₄₀

wherein:
Xaa₁ is: His, dH, or is absent;
Xaa₂ is: dA, Ser, Val, Gly, Thr, Leu, dS, Pro, or Aib;
Xaa₃ is: Asp or Glu;
Xaa₄ is: Ala, Ile, Tyr, Phe, Val, Thr, Leu, Trp, Gly, dA, Aib, or NMeA;
Xaa₅ is: Val, Leu, Phe, Ile, Thr, Trp, Tyr, dV, Aib, or NMeV;
Xaa₆ is: Phe, Ile, Leu, Thr, Val, Trp, or Tyr;
Xaa₈ is: Asp, Glu, Ala, Lys, Leu, Arg, or Tyr;
Xaa₉ is: Asn, Gln, Asp, or Glu;
Xaa₁₀ is: Tyr, Trp, or Tyr(OMe);
Xaa₁₂ is: Arg, Lys, Glu, hR, Orn, Lys (isopropyl), Aib, Cit, or Ala;
Xaa₁₃ is: Leu, Phe, Glu, Ala, or Aib;
Xaa₁₄ is: Arg, Leu, Lys, Ala, hR, Orn, Lys (isopropyl), Phe, Gln, Aib, or Cit;
Xaa₁₅ is: Lys, Ala, Arg, Glu, Leu, hR, Orn, Lys (isopropyl), Phe, Gln, Aib, K(Ac), or Cit;
Xaa₁₆ is: Gln, Lys, Glu, Ala, hR, Orn, Lys (isopropyl), or Cit;
Xaa₁₇ is: Val, Ala, Leu, Ile, Met, Nle, Lys, or Aib;
Xaa₁₉ is: Val, Ala, Glu, Phe, Gly, His, Ile, Lys, Leu, Met, Asn, Pro, Gln, Arg, Ser, Thr, Trp, Tyr, Cys, or Asp;
Xaa₂₀ is: Lys, Gln, hR, Arg, Ser, His, Orn, Lys (isopropyl), Ala, Aib, Trp, Thr, Leu, Ile, Phe, Tyr, Val, K(Ac), or Cit;
Xaa₂₁ is: Lys, His, Arg, Ala, Phe, Aib, Leu, Gln, Orn, hR, K(Ac) or Cit;
Xaa₂₂ is: Tyr, Trp, Phe, Thr, Leu, Ile, Val, Tyr(OMe), Ala, or Aib;
Xaa₂₃ is: Leu, Phe, Ile, Ala, Trp, Thr, Val, or Aib;
Xaa₂₄ is: Gln, Glu, or Asn;
Xaa₂₅ is: Ser, Asp, Phe, Ile, Leu, Thr, Val, Trp, Gln, Asn, Tyr, Aib, or Glu;
Xaa₂₆ is: Ile, Leu, Thr, Val, Trp, Tyr, Phe or Aib;
Xaa₂₇ is: Lys, hR, Arg, Gln, Ala, Asp, Glu, Phe, Gly, His, Ile, Met, Asn, Pro, Ser, Thr, Val, Trp, Tyr, Lys (isopropyl), Cys, Leu, Orn, or dK;
Xaa₂₈ is: Asn, Asp, Gln, Lys, Arg, Aib, Orn, hR, Cit, Pro, or dK;
Xaa₂₉ is: Lys, Ser, Arg, Asn, hR, Ala, Asp, Glu, Phe, Gly, His, Ile, Leu, Met, Pro, Gln, Thr, Val, Trp, Tyr, Cys, Orn, Cit, Aib or is absent;
Xaa₃₀ is: Arg, Lys, Ile, Ala, Asp, Glu, Phe, Gly, His, Leu, Met, Asn, Pro, Gln, Ser, Thr, Val, Trp, Tyr, Cys, hR, Cit, Aib, Orn, or is absent;
Xaa₃₁ is: Tyr, His, Phe, Thr, Cys, or is absent;
Xaa₃₂ is: Ser, Cys, or is absent;
Xaa₃₃ is: Trp or is absent;
Xaa₃₄ is: Cys or is absent;
Xaa₃₅ is: Glu or is absent;
Xaa₃₆ is: Pro or is absent;
Xaa₃₇ is: Gly or is absent;
Xaa₃₈ is: Trp or is absent;
Xaa₃₉ is: Cys or is absent; and
Xaa₄₀ is: Arg or is absent

provided that if Xaa₂₉, Xaa₃₀, Xaa₃₁, Xaa₃₂, Xaa₃₃, Xaa₃₄, Xaa₃₅, Xaa₃₆, Xaa₃₇, Xaa₃₈, or Xaa₃₉ is absent, the next amino acid present downstream is the next amino acid in the peptide agonist sequence,

and a C-terminal extension wherein the N-terminus of the C-terminal extension is linked to the C-terminus of the peptide of Formula 10 and wherein the C-terminal extension comprises an amino acid sequence of the formula:

Formula 11 (SEQ ID NO: 19)
Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂-Xaa₁₃

wherein:
Xaa₁ is: Gly, Cys, or absent;
Xaa₂ is: Gly, Arg, Cys, or absent;
Xaa₃ is: Pro, Thr, Ser, Ala, Cys, or absent;
Xaa₄ is: Ser, Pro, His, Cys, or absent;
Xaa₅ is: Ser, Arg, Thr, Trp, Lys, Cys, or absent;
Xaa₆ is: Gly, Ser, Cys, or absent;
Xaa₇ is: Ala, Asp, Arg, Glu, Lys, Gly, Cys, or absent;
Xaa₈ is: Pro, Ser, Ala, Cys, or absent;
Xaa₉ is: Pro, Ser, Ala, Cys, or absent;
Xaa₁₀ is: Pro, Ser, Ala, Arg, Lys, His, Cys, or absent;
Xaa₁₁ is: Ser, Cys, His, Pro, Lys, Arg, or absent;
Xaa₁₂ is: His, Ser, Arg, Lys, or absent; and
Xaa₁₃ is: His, Ser, Arg, Lys, or absent;

provided that at least five of Xaa₁ to Xaa₁₃ of the C-terminal extension are present and provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ is absent, the next amino acid present downstream is the next amino acid in the C-terminal extension and wherein the C-terminal amino acid may be amidated.

Preferably, at least six of Xaa₁ to Xaa₁₃ of the C-terminal extension are present. More preferably at least seven, eight, nine ten, eleven, twelve or all of Xaa₁ to Xaa₁₃ of the C-terminal extension are present.

Preferably, the VPAC2 receptor peptide agonist comprises a sequence of the formula:

Formula 12 (SEQ ID NO: 20)
Xaa₁-Xaa₂-Xaa₃-Xaa₄-Xaa₅-Xaa₆-Thr-Xaa₈-Xaa₉-Xaa₁₀-
Thr-Xaa₁₂-Xaa₁₃-Xaa₁₄-Xaa₁₅-Xaa₁₆-Xaa₁₇-Ala-Xaa₁₉-
Xaa₂₀-Xaa₂₁-Xaa₂₂-Xaa₂₃-Xaa₂₄-Xaa₂₅-Xaa₂₆-Xaa₂₇-
Xaa₂₈-Xaa₂₉-Xaa₃₀-Xaa₃₁-Xaa₃₂

wherein:
Xaa₁ is: His, dH, or is absent;
Xaa₂ is: dA, Ser, Val, Gly, Thr, Leu, dS, Pro, or Aib;
Xaa₃ is: Asp or Glu;
Xaa₄ is: Ala, Ile, Tyr, Phe, Val, Thr, Leu, Trp, Gly, dA, Aib, or NMeA;
Xaa₅ is: Val, Leu, Phe, Ile, Thr, Trp, Tyr, dV, Aib, or NMeV;
Xaa₆ is: Phe, Ile, Leu, Thr, Val, Trp, or Tyr;
Xaa₈ is: Asp, Glu, Ala, Lys, Leu, Arg, or Tyr;
Xaa₉ is: Asn, Gln, or Glu;
Xaa₁₀ is: Tyr, Trp, or Tyr(OMe);
Xaa₁₂ is: Arg, Lys, hR, Orn, Aib, Cit, or Ala;
Xaa₁₃ is: Leu, Phe, Glu, Ala, or Aib;
Xaa₁₄ is: Arg, Leu, Lys, Ala, hR, Orn, Phe, Gln, Aib, or Cit;
Xaa₁₅ is: Lys, Ala, Arg, Glu, Leu, hR, Orn, Phe, Gln, Aib, K(Ac), or Cit;
Xaa₁₆ is: Gln, Lys, Ala, hR, Orn, or Cit;
Xaa₁₇ is: Val, Ala, Leu, Ile, Met, Nle, Lys, or Aib;
Xaa₁₉ is: Ala, Gly, or Leu;
Xaa₂₀ is: Lys, Gln, hR, Arg, Ser, Orn, Ala, Aib, Trp, Thr, Leu, Ile, Phe, Tyr, Val, K(Ac), or Cit;
Xaa₂₁ is: Lys, Arg, Ala, Phe, Aib, Leu, Gln, Orn, hR, K(Ac) or Cit;
Xaa₂₂ is: Tyr, Trp, Phe, Thr, Leu, Ile, Val, Tyr(OMe), Ala, or Aib;
Xaa₂₃ is: Leu, Phe, Ile, Ala, Trp, Thr, Val, or Aib;
Xaa₂₄ is: Gln, or Asn;
Xaa₂₅ is: Ser, Asp, Phe, Ile, Leu, Thr, Val, Trp, Gln, Asn, Tyr, Aib, or Glu;
Xaa₂₆ is: Ile, Leu, Thr, Val, Trp, Tyr, Phe or Aib;
Xaa₂₇ is: Lys, hR, Arg, Gln, Orn, or dK;
Xaa₂₈ is: Asn, Gln, Lys, Arg, Aib, Orn, hR, Cit, Pro, or dK;
Xaa₂₉ is: Lys, Ser, Arg, Asn, hR, Orn, Cit, Aib or is absent;
Xaa₃₀ is: Arg, Lys, Ile, hR, Cit, Aib, Orn, or is absent;
Xaa₃₁ is: Tyr, His, Phe, or is absent; and
Xaa₃₂ is: Cys, or is absent;

provided that if Xaa₂₉, Xaa₃₀, or Xaa₃₁ is absent, the next amino acid present downstream is the next amino acid in the peptide agonist sequence,

and a C-terminal extension wherein the N-terminus of the C-terminal extension is linked to the C-terminus of the peptide of Formula 12 and wherein the C-terminal extension comprises an amino acid sequence of the formula:

Formula 11 (SEQ ID NO: 19)
Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂-Xaa₁₃

wherein:
Xaa₁ is: Gly, Cys, or absent;
Xaa₂ is: Gly, Arg, Cys, or absent;
Xaa₃ is: Pro, Thr, Ser, Ala, Cys, or absent;
Xaa₄ is: Ser, Pro, His, Cys, or absent;
Xaa₅ is: Ser, Arg, Thr, Trp, Lys, Cys, or absent;
Xaa₆ is: Gly, Ser, Cys, or absent;
Xaa₇ is: Ala, Asp, Arg, Glu, Lys, Gly, Cys, or absent;
Xaa₈ is: Pro, Ser, Ala, Cys, or absent;
Xaa₉ is: Pro, Ser, Ala, Cys, or absent;
Xaa₁₀ is: Pro, Ser, Ala, Arg, Lys, His, Cys, or absent;
Xaa₁₁ is: Ser, Cys, His, Pro, Lys, Arg, or absent;
Xaa₁₂ is: His, Ser, Arg, Lys, or absent; and
Xaa₁₃ is: His, Ser, Arg, Lys, or absent;

provided that at least five of Xaa₁ to Xaa₁₃ of the C-terminal extension are present and provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ is absent, the next amino acid present downstream is the next amino acid in the C-terminal extension and wherein the C-terminal amino acid may be amidated.

The VPAC2 receptor peptide agonist preferably comprises a sequence of the formula:

Formula 13 (SEQ ID NO: 21)
His-Xaa₂-Xaa₃-Xaa₄-Xaa₅-Phe-Thr-Xaa₈-Xaa₉-Tyr-Thr-
Xaa₁₂-Leu-Xaa₁₄-Xaa₁₅-Xaa₁₆-Xaa₁₇-Ala-Xaa₁₉-Xaa₂₀-
Xaa₂₁-Xaa₂₂-Leu-Xaa₂₄-Xaa₂₅-Xaa₂₆-Xaa₂₇-Xaa₂₈-
Xaa₂₉-Xaa₃₀-Xaa₃₁

wherein:
Xaa₂ is: dA, Ser, Val, dS, or Aib;
Xaa₃ is: Asp or Glu;
Xaa₄ is: Ala, dA, or Aib;
Xaa₅ is: Val, Leu, dV, or Aib;
Xaa₈ is: Asp, Glu, or Ala;
Xaa₉ is: Asn, Gln, or Glu;
Xaa₁₂ is: Ala, Arg, Lys, hR, or Orn;
Xaa₁₄ is: Arg, Leu, Lys, Ala, hR, Orn, Phe, Gln, Aib, or Cit;
Xaa₁₅ is: Lys, Ala, Arg, Leu, Orn, Phe, Gln, Aib, or K(Ac);
Xaa₁₆ is: Gln, or Lys;
Xaa₁₇ is: Val, Ala, Leu, Ile, Met, Nle, or Lys;
Xaa₁₉ is: Ala, or Leu;
Xaa₂₀ is: Lys, Gln, hR, Arg, Ser, Ala, Aib, Trp, Thr, Leu, Ile, Phe, Tyr, Val, or K(Ac);
Xaa₂₁ is: Lys, Arg, Ala, Phe, Aib, Leu, Gln, K(Ac), or Orn;
Xaa₂₂ is: Tyr, Trp, Phe, Leu, Ile, or Val;
Xaa₂₄ is: Gln, or Asn;
Xaa₂₅ is: Ser, Phe, Ile, Leu, Thr, Val, Trp, Gln, Asn, Tyr, or Aib;
Xaa₂₆ is: Ile, Leu, Thr, Val, Trp, Tyr, Phe or Aib;
Xaa₂₇ is: Lys, hR, Arg, dK, or Orn;
Xaa₂₈ is: Asn, Gln, Lys, hR, Aib, Orn, dK, or Pro;
Xaa₂₉ is: Lys, Ser, Arg, hR, Orn, or is absent;
Xaa₃₀ is: Arg, Lys, or is absent; and
Xaa₃₁ is: Tyr, Phe, or is absent;

provided that if Xaa₂₉, or Xaa₃₀ is absent, the next amino acid present downstream is the next amino acid in the peptide agonist sequence,

and a C-terminal extension wherein the N-terminus of the C-terminal extension is linked to the C-terminus of the peptide of Formula 13 and wherein the C-terminal extension comprises an amino acid sequence of the formula:

Formula 11 (SEQ ID NO: 19)
Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂-Xaa₁₃

wherein:
Xaa₁ is: Gly, Cys, or absent;
Xaa₂ is: Gly, Arg, Cys, or absent;
Xaa₃ is: Pro, Thr, Ser, Ala, Cys, or absent;
Xaa₄ is: Ser, Pro, His, Cys, or absent;
Xaa₅ is: Ser, Arg, Thr, Trp, Lys, Cys, or absent;
Xaa₆ is: Gly, Ser, Cys, or absent;
Xaa₇ is: Ala, Asp, Arg, Glu, Lys, Gly, Cys, or absent;
Xaa₈ is: Pro, Ser, Ala, Cys, or absent;
Xaa₉ is: Pro, Ser, Ala, Cys, or absent;
Xaa₁₀ is: Pro, Ser, Ala, Arg, Lys, His, Cys, or absent;
Xaa₁₁ is: Ser, Cys, His, Pro, Lys, Arg, or absent;
Xaa₁₂ is: His, Ser, Arg, Lys, or absent; and
Xaa₁₃ is: His, Ser, Arg, Lys, or absent;

provided that at least five of Xaa₁ to Xaa₁₃ of the C-terminal extension are present and provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ is absent, the next amino acid present downstream is the next amino acid in the C-terminal extension and wherein the C-terminal amino acid may be amidated.

Preferably, the VPAC2 receptor peptide agonist sequence further comprises a histidine residue at the N-terminal extension region of the peptide sequence before Xaa₁.

Preferably, the C-terminal extension of the VPAC2 receptor peptide agonist comprises an amino acid sequence of the formula:

Formula 7 (SEQ ID NO: 15)
Xaa₁-Xaa₂-Xaa₃-Xaa₄-Xaa₅-Xaa₆-Xaa₇-Xaa₈-Xaa₉-
Xaa₁₀-Xaa₁₁

wherein:
Xaa₁ is: Gly, Cys, or absent;
Xaa₂ is: Gly, Arg, or absent;
Xaa₃ is: Pro, Thr, or absent;
Xaa₄ is: Ser, or absent;
Xaa₅ is: Ser, or absent;
Xaa₆ is: Gly, or absent;
Xaa₇ is: Ala, or absent;
Xaa₈ is: Pro, or absent;
Xaa₉ is: Pro, or absent;
Xaa₁₀ is: Pro, or absent; and
Xaa₁₁ is: Ser, Cys, or absent;

provided that at least five of Xaa₁ to Xaa₁₁ of the C-terminal extension are present and provided that if Xaa₁, Xaa₂, Xaa₃, Xaa₄, Xaa₅, Xaa₆, Xaa₇, Xaa₈, Xaa₉, or Xaa₁₀ is absent, the next amino acid present downstream is the next amino acid in the C-terminal extension and wherein the C-terminal amino acid may be amidated.

More preferably, the C-terminal extension of the VPAC2 receptor peptide agonist is selected from:

| | |
|---|---|
| SEQ ID NO: 10 | GGPSSGAPPPS |
| SEQ ID NO: 11 | GGPSSGAPPPS-NH₂ |
| SEQ ID NO: 22 | GGPSSGAPPPC |
| SEQ ID NO: 23 | GGPSSGAPPPC-NH₂ |
| SEQ ID NO: 24 | GRPSSGAPPPS |
| SEQ ID NO: 25 | GRPSSGAPPPS-NH₂ |

It is especially preferred that the C-terminal extension is GGPSSGAPPPS (SEQ ID NO: 10) or GGPSSGAPPPS-NH₂ (SEQ ID NO: 11).

Preferably, the VPAC2 receptor peptide agonist of the present invention comprises a sequence of the Formula 10 (SEQ ID NO. 18), Formula 12 (SEQ ID NO 20) or Formula 13 (SEQ ID NO 21) wherein Xaa₃ is Asp or Glu, Xaa₈ is Asp or Glu, Xaa₁₂ is Arg, hR, Lys, or Orn, Xaa₁₄ is Arg, Gln, Aib, hR, Orn, Cit, Lys, Ala, or Leu, Xaa₁₅ is Lys, Aib, or Orn, Xaa₁₆ is Gln or Lys, Xaa₁₇ is Val, Leu, Ala, Ile, Lys, or Nle, Xaa₂₀ is Lys, Val, Leu, Aib, Ala, or Gln, Xaa₂₁ is Lys, Aib, Orn, Ala, or Gln, Xaa₂₇ is Lys, Orn, or hR, Xaa₂₈ is Asn, Gln, Lys, hR, Aib, Orn, or Pro and Xaa₂₉ is Lys, Orn, hR, or is absent.

More preferably, the VPAC2 receptor peptide agonist of the present invention comprises a sequence of the Formula 10 (SEQ ID NO. 18), Formula 12 (SEQ ID NO 20) or Formula 13 (SEQ ID NO 21) wherein Xaa₁₂ is Arg, hR, or Orn, Xaa₁₄ is Arg, Aib, Gln, Ala, Leu, Lys, or Orn, Xaa₁₅ is Lys or Aib, Xaa₁₇ is Val or Leu, Xaa₂₁ is Lys, Aib, or Gln and Xaa₂₈ is Asn or Gln.

Preferably, the VPAC2 receptor peptide agonist of the present invention comprises a sequence of the Formula 10 (SEQ ID NO. 18), Formula 12 (SEQ ID NO 20) or Formula 13 (SEQ ID NO 21) wherein Xaa₃₀ and/or Xaa₃₁ are absent. Alternatively, Xaa₂₉, Xaa₃₀ and Xaa₃₁ are all absent.

Preferably, the VPAC2 receptor peptide agonist of the present invention comprises a sequence of the Formula 10 (SEQ ID NO. 18), Formula 12 (SEQ ID NO 20) or Formula 13 (SEQ ID NO 21) wherein either Xaa₁₄ or Xaa₁₅ is Aib.

Also preferably, the VPAC2 receptor peptide agonist of the present invention comprises a sequence of the Formula 10 (SEQ ID NO. 18), Formula 12 (SEQ ID NO 20) or Formula 13 (SEQ ID NO 21) wherein either Xaa₂₀ or Xaa₂₁ is Aib.

More preferably, either Xaa₁₄ or Xaa₁₅ is Aib and either Xaa₂₀ or Xaa₂₁ is Aib.

Preferably, the VPAC2 receptor peptide agonist of the present invention comprises a sequence of the Formula 10 (SEQ ID NO. 18), Formula 12 (SEQ ID NO 20) or Formula 13 (SEQ ID NO 21) wherein Xaa₂₈ is Gln and Xaa₂₉ is Lys or is absent.

More preferably, Xaa₂₈ is Gln and Xaa₂₉ is Lys or is absent, and either Xaa₁₄ or Xaa₁₅ is Aib and either Xaa₂₀ or Xaa₂₁ is Aib.

Preferably, the VPAC2 receptor peptide agonist of the present invention comprises a sequence of the Formula 10 (SEQ ID NO. 18), Formula 12 (SEQ ID NO 20) or Formula 13 (SEQ ID NO 21) wherein Xaa₁₂ is hR or Orn, Xaa₂₇ is hR or Orn and Xaa₂₉ is hR or Orn.

More preferably, Xaa₁₂ is hR or Orn, Xaa₂₇ is hR or Orn and Xaa₂₉ is hR or Orn, and either Xaa₁₄ or Xaa₁₅ is Aib and either Xaa₂₀ or Xaa₂₁ is Aib.

Preferably, the VPAC2 receptor peptide agonist of the present invention further comprises a N-terminal modification at the N-terminus of the peptide agonist wherein the N-terminal modification is selected from:
a) addition of D-histidine, isoleucine, methionine, or norleucine;
b) addition of a peptide comprising the sequence Ser-Trp-Cys-Glu-Pro-Gly-Trp-Cys-Arg wherein the Arg is linked to the N-terminus of the peptide agonist;
c) addition of C₁-C₁₆ alkyl optionally substituted with one or more substituents independently selected from aryl, C₁-C₆ alkoxy, —NH₂, —OH, halogen and —CF₃;
d) addition of —C(O)R¹ wherein R¹ is a C₁-C₁₆ alkyl optionally substituted with one or more substituents independently selected from aryl, C₁-C₆ alkoxy, —NH₂, —OH, halogen, —SH and —CF₃; an aryl or aryl C₁-C₄ alkyl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃; —NR²R³ wherein R² and R³ are independently hydrogen, C₁-C₆ alkyl, aryl or aryl C₁-C₄ alkyl; or —OR⁴ wherein R⁴ is C₁-C₁₆ alkyl optionally substituted with one or more substituents independently selected from aryl, C₁-C₆ alkoxy, —NH₂, —OH, halogen and —CF₃, aryl or aryl C₁-C₄ alkyl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃;
e) addition of —SO₂R⁵ wherein R⁵ is aryl, aryl C₁-C₄ alkyl or C₁-C₁₆ alkyl;
f) formation of a succinimide group optionally substituted with C₁-C₆ alkyl or —SR⁶, wherein R⁶ is hydrogen or C₁-C₆ alkyl; and
g) addition of methionine sulfoxide.

Preferably, the N-terminal modification is the addition of a group selected from: acetyl, propionyl, butyryl, pentanoyl, hexanoyl, methionine, methionine sulfoxide, 3-phenylpropionyl, phenylacetyl, benzoyl, norleucine, D-histidine, isoleucine and 3-mercaptopropionyl and more preferably is the addition of acetyl or hexanoyl. It is especially preferred that the N-terminal modification is the addition of hexanoyl.

It will be appreciated by the skilled person that various combinations of the VPAC2 receptor peptide agonist sequence, C-terminal extension sequence and N-terminal modifications described above may be made based on the above disclosure.

According to a second aspect of the present invention, the preferred VPAC2 receptor peptide agonists comprise an amino acid sequence selected from:

| Agonist | |
|---|---|
| # | Sequence |
| P6 | HSDAVFTDNYTRLRKQVAAKKYLQSIKNKRYGGPSSGAPPPS |
| SEQ ID NO: 28 | |
| P7 | HSDAVFTDNYTRLRKQVAAKKYLQSIKNKRGGT |
| SEQ ID NO: 29 | |
| P8 | HSDAVFTDNYTRLRKQVAAKKYLQSIKNKKGGT |
| SEQ ID NO: 30 | |
| P9 | HSDAVFTDNYTRLRKQVAAKKYLQSIKNKKGGPSSGAPPPS |
| SEQ ID NO: 31 | |
| P18 | HSDAVFTDNYTRLRKQVAAhRKYLQSIKNKRYGGPSSGAPPPS |
| SEQ ID NO: 32 | |
| P19 | HSDAVFTDNYTRLRKQVAAIKYLQSIKNKRYGGPSSGAPPPS |
| SEQ ID NO: 33 | |
| P20 | HSDAVFTDNYTRLRKQVAARKYLQSIKNKRYGGPSSGAPPPS |
| SEQ ID NO: 34 | |
| P21 | HSDAVFTDNYTRLRKQVAASKYLQSIKNKRYGGPSSGAPPPS |
| SEQ ID NO: 35 | |
| P22 | HSDAVFTDNYTRLRKQVAAKKYLQSIhRNKRYGGPSSGAPPPS |
| SEQ ID NO: 36 | |
| P23 | HSDAVFTDNYTRLRKQVAAKKYLQSIRNKRYGGPSSGAPPPS |
| SEQ ID NO: 37 | |
| P24 | HSDAVFTDNYTRLRKQVAAKKYLQSIKNhRRYGGPSSGAPPPS |
| SEQ ID NO: 38 | |
| P25 | HSDAVFTDNYTRLRKQVAAKKYLQSIKNRRYGGPSSGAPPPS |
| SEQ ID NO: 39 | |
| P26 | HSDAVFTDNYTRFRKQVAAKKYLQSIKNKRYGGPSSGAPPPS |
| SEQ ID NO: 40 | |
| P27 | HSDAVFTDNWTRLRKQVAAKKYLQSIKNKRYGGPSSGAPPPS |
| SEQ ID NO: 41 | |
| P28 | HSDAVFTDNYTRLRKQVAAKKYLQSIKNKRHGGPSSGAPPPS |
| SEQ ID NO: 42 | |
| P31 | C6-HSDAVFTDNYTRLRKQVAAKKYLQSIKNKRYGGPSSGAPPPS |
| SEQ ID NO: 43 | |
| P33 | Ac-HSDAVFTDNYTRLRKQVAAKKYLQSIKNKRYGGPSSGAPPPS |
| SEQ ID NO: 44 | |
| P34 | HSDAVFTDNYTRLRKQVAAKKYLQSIKNKRF |
| SEQ ID NO: 45 | |
| P37 | HSDAVFTDNYTRLRKQVAAKKYLQSIKNKRYC |
| SEQ ID NO: 46 | |
| P43 | HGDAVFTDNYTRLRKQVAAKKYLQSIKNKRYGGPSSGAPPPS |
| SEQ ID NO: 47 | |
| P44 | HVDAVFTDNYTRLRKQVAAKKYLQSIKNKRYGGPSSGAPPPS |
| SEQ ID NO: 48 | |
| P45 | HTDAVFTDNYTRLRKQVAAKKYLQSIKNKRYGGPSSGAPPPS |
| SEQ ID NO: 49 | |
| P46 | HLDAVFTDNYTRLRKQVAAKKYLQSIKNKRYGGPSSGAPPPS |
| SEQ ID NO: 50 | |
| P47 | HdADAVFTDNYTRLRKQVAAKKYLQSIKNKRYGGPSSGAPPPS |
| SEQ ID NO: 51 | |
| P48 | HdSDAVFTDNYTRLRKQVAAKKYLQSIKNKRYGGPSSGAPPPS |
| SEQ ID NO: 52 | |
| P49 | HPDAVFTDNYTRLRKQVAAKKYLQSIKNKRYGGPSSGAPPPS |
| SEQ ID NO: 53 | |
| P50 | HSDIVFTDNYTRLRKQVAAKKYLQSIKNKRYGGPSSGAPPPS |
| SEQ ID NO: 54 | |
| P51 | HSDYVFTDNYTRLRKQVAAKKYLQSIKNKRYGGPSSGAPPPS |
| SEQ ID NO: 55 | |
| P52 | HSDFVFTDNYTRLRKQVAAKKYLQSIKNKRYGGPSSGAPPPS |
| SEQ ID NO: 56 | |
| P53 | HSDVVFTDNYTRLRKQVAAKKYLQSIKNKRYGGPSSGAPPPS |
| SEQ ID NO: 57 | |
| ID58 | |
| P54 | HSDTVFTDNYTRLRKQVAAKKYLQSIKNKRYGGPSSGAPPPS |
| SEQ NO: 58 | |
| P55 | HSDLVFTDNYTRLRKQVAAKKYLQSIKNKRYGGPSSGAPPPS |
| SEQ ID NO: 59 | |
| P56 | HSDWVFTDNYTRLRKQVAAKKYLQSIKNKRYGGPSSGAPPPS |
| SEQ ID NO: 60 | |
| P58 | HSDAFFTDNYTRLRKQVAAKKYLQSIKNKRYGGPSSGAPPPS |
| SEQ ID NO: 61 | |
| P60 | HSDAIFTDNYTRLRKQVAAKKYLQSIKNKRYGGPSSGAPPPS |
| SEQ ID NO: 62 | |
| P61 | HSDALFTDNYTRLRKQVAAKKYLQSIKNKRYGGPSSGAPPPS |
| SEQ ID NO: 63 | |
| P62 | HSDATFTDNYTRLRKQVAAKKYLQSIKNKRYGGPSSGAPPPS |
| SEQ ID NO: 64 | |
| P63 | HSDAVITDNYTRLRKQVAAKKYLQSIKNKRYGGPSSGAPPPS |
| SEQ ID NO: 65 | |
| P64 | HSDAVLTDNYTRLRKQVAAKKYLQSIKNKRYGGPSSGAPPPS |
| SEQ ID NO: 66 | |
| P65 | HSDAVTTDNYTRLRKQVAAKKYLQSIKNKRYGGPSSGAPPPS |
| SEQ ID NO: 67 | |
| P66 | HSDAVVTDNYTRLRKQVAAKKYLQSIKNKRYGGPSSGAPPPS |
| SEQ ID NO: 68 | |
| P67 | HSDAVWTDNYTRLRKQVAAKKYLQSIKNKRYGGPSSGAPPPS |
| SEQ ID NO: 69 | |
| P68 | HSDAVYTDNYTRLRKQVAAKKYLQSIKNKRYGGPSSGAPPPS |
| SEQ ID NO: 70 | |
| P69 | HSDAWFTDNYTRLRKQVAAKKYLQSIKNKRYGGPSSGAPPPS |
| SEQ ID NO: 71 | |
| P70 | HSDAYFTDNYTRLRKQVAAKKYLQSIKNKRYGGPSSGAPPPS |
| SEQ ID NO: 72 | |
| P71 | HSDAVFTDNYTRLRRQVAARRYLQSIRNRRYGGPSSGAPPPS |
| SEQ ID NO: 73 | |
| P72 | HSDAVFTDNYTRLRRQVAAKKYLQSIKNKRYGGPSSGAPPPS |
| SEQ ID NO: 74 | |
| P73 | HSDAVFTDNYTRLRKQVAARKYLQSIKNKRYGGPSSGAPPPS |
| SEQ ID NO: 75 | |
| P74 | HSDAVFTDNYTRLRKQVAAKKYLQSIQNKRYGGPSSGAPPPS |
| SEQ ID NO: 76 | |
| P75 | HSDAVFTDNYTRLRKQVAAKKYLQSIKNNRYGGPSSGAPPPS |
| SEQ ID NO: 77 | |
| P76 | HSDAVFTDNYTRLRKQVAAKKYLQSIKNKIYGGPSSGAPPPS |
| SEQ ID NO: 78 | |
| P82 | HSDAVFTDNYTRLRKQVAAKKYLQSIKRGGPSSGAPPPS |
| SEQ ID NO: 79 | |
| P83 | HSDAVFTDNYTRLRKQVAAKKYLQSIKNGGPSSGAPPPS |
| SEQ ID NO: 80 | |
| P84 | dHSDAVFTDNYTRLRKQVAAKKYLQSIKNKRYGGPSSGAPPPS |
| SEQ ID NO: 81 | |
| P85 | HSDAVFTDNYTRLRKQVAAKKYLQSIKKGGPSSGAPPPS |
| SEQ ID NO: 82 | |
| P87 | HSDAVFTDNYTREKEKVAAKKYLQSIKNKRYGGPSSGAPPPS |
| SEQ ID NO: 83 | |
| P88 | HSDAVFTDNYTRAAAKVAAKKYLQSIKNKRYGGPSSGAPPPS |
| SEQ ID NO: 84 | |
| P89 | HSDAVFTDNYTRLLAKVAAKKYLQSIKNKRYGGPSSGAPPPS |
| SEQ ID NO: 85 | |
| P92 | HSDAVFTDNYTRLRKQVAAKKYLQSIKNGRPSSGAPPPS |
| SEQ ID NO: 86 | |
| P93 | HSDAVFTDNYTRLLLKVAAKKYLQSIKNKRYGGPSSGAPPPS |
| SEQ ID NO: 87 | |
| P94 | HSDAVFTDNYTRAKAKVAAKKYLQSIKNKRYGGPSSGAPPPS |
| SEQ ID NO: 88 | |
| P98 | C6-HSDAVFTDNYTRLRRQVAARRYLQSIRNRRYGGPSSGAPPPS |
| SEQ ID NO: 89 | |
| P99 | C6-HVDAVFTDNYTRLRKQVAAKKYLQSIKNKRYGGPSSGAPPPS |
| SEQ ID NO: 90 | |
| P100 | M-HSDAVFTDQYTRLRKQVAAKKYLQSIKQKRYGGPSSGAPPPS |
| SEQ ID NO: 91 | |
| P101 | C6-HdADAVFTDNYTRLRKQVAAKKYLQSIKNKRYGGPSSGAPPPS |
| SEQ ID NO: 92 | |
| P102 | HSDGVFTDNYTRLRKQVAAKKYLQSIKNKRYGGPSSGAPPPS |
| SEQ ID NO: 93 | |
| P103 | C6-HSDAVFTDNYTKLKKQVAAKKYLQSIKNKKYGGPSSGAPPPS |
| SEQ ID NO: 94 | |
| P104 | M-HSDAVFTDNYTRLRKQVAAKKYLQSIKNKRYGGPSSGAPPPS |
| SEQ ID NO: 95 | |
| P105 | I-HSDAVFTDNYTRLRKQVAAKKYLQSIKNKRYGGPSSGAPPPS |
| SEQ ID NO: 96 | |
| P106 | C6-HSDAVFTDNYTRLRKQVAAKKYLQSFKNKRYGGPSSGAPPPS |
| SEQ ID NO: 97 | |
| P107 | C6-HSDAVFTDNYTRLRKQVAAKKYLQSLKNKRYGGPSSGAPPPS |
| SEQ ID NO: 98 | |
| P108 | C6-HSDAVFTDNYTRLRKQVAAKKYLQSTKNKRYGGPSSGAPPPS |
| SEQ ID NO: 99 | |
| P109 | C6-HSDAVFTDNYTRLRKQVAAKKYLQSVKNKRYGGPSSGAPPPS |
| SEQ ID NO: 100 | |
| P110 | C6-HSDAVFTDNYTRLRKQVAAKKYLQSWKNKRYGGPSSGAPPPS |
| SEQ ID NO: 101 | |
| P111 | C6-HSDAVFTDNYTRLRKQVAAKKYLQSYKNKRYGGPSSGAPPPS |
| SEQ ID NO: 102 | |
| P112 | C6-HSDAVFTDNYTRLRKQVAAKKYLQFIKNKRYGGPSSGAPPPS |
| SEQ ID NO: 103 | |
| P113 | C6-HSDAVFTDNYTRLRKQVAAKKYLQIIKNKRYGGPSSGAPPPS |
| SEQ ID NO: 104 | |
| P114 | C6-HSDAVFTDNYTRLRKQVAAKKYLQLIKNKRYGGPSSGAPPPS |
| SEQ ID NO: 105 | |
| P115 | C6-HSDAVFTDNYTRLRKQVAAKKYLQTIKNKRYGGPSSGAPPPS |
| SEQ ID NO: 106 | |
| P116 | C6-HSDAVFTDNYTRLRKQVAAKKYLQVIKNKRYGGPSSGAPPPS |
| SEQ ID NO: 107 | |
| P117 | C6-HSDAVFTDNYTRLRKQVAAKKYLQWIKNKRYGGPSSGAPPPS |
| SEQ ID NO: 108 | |
| P119 | C6-HSDAVFTDNYTRLLAKLALQKYLQSIKNKRYGGPSSGAPPPS |
| SEQ ID NO: 109 | |
| P120 | C6-HSDAVFTDNYTRLRKQVAAKKYLQSIKNKRYGGPSSGAPPPC |
| SEQ ID NO: 110 | |
| P121 | C6-HSDAVFTDNYTRLRKQVAAKKYLQSIKNKKGGPSSGAPPPS |
| SEQ ID NO: 111 | |
| P122 | C6-HSDAVFTDNYTRLRKQVAAKKYLQSIKNSRGGPSSGAPPPS |
| SEQ ID NO: 112 | |
| P123 | C6-HSDAVFTDNYTRLRKQVAAKKYLQSIKNKRGGPSSGAPPPS |
| SEQ ID NO: 113 | |
| P124 | C6-HSDAVFTDNYTRLRKQVAAKKYLQQIKNKRYGGPSSGAPPPS |
| SEQ ID NO: 114 | |
| P125 | C6-HSDAVFTDNYTRLRKQVAAKKYLQNIKNKRYGGPSSGAPPPS |
| SEQ ID NO: 115 | |
| P126 | HSDAVFTDNYTRLRKQVAAKKYLQSIKRGRPSSGAPPPS |
| SEQ ID NO: 116 | |
| P127 | C6-HSDAVFTDNYTRLRKQVAAKKYLQYIKNKRYGGPSSGAPPPS |
| SEQ ID NO: 117 | |
| P129 | C6-HSDAVFTDNYTRLRKQVAAKKWLQSIKNKRYGGPSSGAPPPS |
| SEQ ID NO: 118 | |
| P130 | C6-HSDAVFTDNYTRLRKQVAAKKFLQSIKNKRYGGPSSGAPPPS |
| SEQ ID NO: 119 | |
| P131 | C6-HSDAVFTDNYTRLRKQVAAKKTLQSIKNKRYGGPSSGAPPPS |
| SEQ ID NO: 120 | |
| P132 | C6-HSDAVFTDNYTRLRKQVAAKKLLQSIKNKRYGGPSSGAPPPS |
| SEQ ID NO: 121 | |
| P133 | C6-HSDAVFTDNYTRLRKQVAAKKILQSIKNKRYGGPSSGAPPPS |
| SEQ ID NO: 122 | |
| P134 | C6-HSDAVFTDNYTRLRKQVAAKKVLQSIKNKRYGGPSSGAPPPS |
| SEQ ID NO: 123 | |
| P135 | C6-HSDAVFTDNYTRLLAKVAAKKYLQSIKNKRYGGPSSGAPPPS |
| SEQ ID NO: 124 | |
| P138 | C6-HSDAVFTDNYTRLRAQVAAQKYLQSIKNKRYGGPSSGAPPPS |
| SEQ ID NO: 125 | |
| P139 | C6-HSDAVFTDNYTRLRAQVAAKKYLQSIKNKRYGGPSSGAPPPS |
| SEQ ID NO: 126 | |
| P140 | M-HSDAVFTDNYTRLLAKLALQKYLQSIKNKRYGGPSSGAPPPS |
| SEQ ID NO: 127 | |
| P141 | C6-HSDAVFTDNYTRLKAQVAAKKYLQSIKNKRYGGPSSGAPPPS |
| SEQ ID NO: 128 | |
| P142 | C6-HSDAVFTDNYTRLRAQLAAQKYLQSIKNKRYGGPSSGAPPPS |
| SEQ ID NO: 129 | |
| P143 | C6-HSDAVFTDNYTRLRKQMAAQKYLNQLKKGGPSSGAPPPS |
| SEQ ID NO: 130 | |
| P144 | C6-HSDAVFTDNYTRLRKQVAAQKYLNQLKKGGPSSGAPPPS |
| SEQ ID NO: 131 | |
| P146 | C6-HSDAVFTDNYTRLRKQVAAVKYLQSIKNKRYGGPSSGAPPPS |
| SEQ ID NO: 132 | |
| P147 | C6-HSDAVFTDNYTRLRKQVAAYKYLQSIKNKRYGGPSSGAPPPS |
| SEQ ID NO: 133 | |
| P148 | C6-HSDAVFTDNYTRLRKQVAAFKYLQSIKNKRYGGPSSGAPPPS |
| SEQ ID NO: 134 | |
| P149 | C6-HSDAVFTDNYTRLRKQVAAIKYLQSIKNKRYGGPSSGAPPPS |
| SEQ ID NO: 135 | |
| P150 | C6-HSDAVFTDNYTRLRKQVAAQKYLQSIKNKRYGGPSSGAPPPS |
| SEQ ID NO: 136 | |
| P151 | C6-HSDAVFTDNYTRLRKQVAALKYLQSIKNKRYGGPSSGAPPPS |
| SEQ ID NO: 137 | |
| P152 | C6-HSDAVFTDNYTRLRKQVAATKYLQSIKNKRYGGPSSGAPPPS |
| SEQ ID NO: 138 | |
| P153 | C6-HSDAVFTDNYTRLRKQVAAWKYLQSIKNKRYGGPSSGAPPPS |
| SEQ ID NO: 139 | |
| P154 | C6-HSDAVFTDNYTRLRKQVAAKKYLQSIKNGGPSSGAPPPS |
| SEQ ID NO: 140 | |
| P155 | C6-HSDAVFTDNYTRLRKQVALKKYLQSIKNKRYGGPSSGAPPPS |
| SEQ ID NO: 141 | |
| P158 | C6-HSDAVFTANYTRLRKQVAAKKYLQSIKNKRYGGPSSGAPPPS |
| SEQ ID NO: 142 | |
| P159 | C6-HSDAVFTENYTRLRKQVAAKKYLQSIKNKRYGGPSSGAPPPS |
| SEQ ID NO: 143 | |
| P160 | C6-HSDAVFTKNYTRLRKQVAAKKYLQSIKNKRYGGPSSGAPPPS |
| SEQ ID NO: 144 | |
| P161 | C6-HSDAVFTLNYTRLRKQVAAKKYLQSIKNKRYGGPSSGAPPPS |
| SEQ ID NO: 145 | |
| P162 | C6-HSDAVFTRNYTRLRKQVAAKKYLQSIKNKRYGGPSSGAPPPS |
| SEQ ID NO: 146 | |
| P163 | C6-HSDAVFTYNYTRLRKQVAAKKYLQSIKNKRYGGPSSGAPPPS |
| SEQ ID NO: 147 | |
| P164 | C6-HSDAVFTDNYTRLLAKLALQKYLQSIKNKRYGGPSSGAPPPC |
| SEQ ID NO: 148 | |
| P165 | C6-HSDAVFTEEYTRLQKQVAAKQYLQSIKNKRYGGPSSGAPPPS |
| SEQ ID NO: 149 | |
| P166 | C6-HAibDAVFTDNYTRLRKQVAAKKYLQSIKNKRYGGPSSGAPPPS |
| SEQ ID NO: 150 | |
| P167 | C6-HSDAVFTDNYTRLAibKQVAAKKYLQSIKNKRYGGPSSGAPPPS |
| SEQ ID NO: 151 | |
| P168 | C6-HSDAVFTDNYTRLRAibQVAAKKYLQSIKNKRYGGPSSGAPPPS |
| SEQ ID NO: 152 | |
| P169 | C6-HSDAVFTDNYTRLRKQVAAAibKYLQSIKNKRYGGPSSGAPPPS |
| SEQ ID NO: 153 | |
| P170 | C6-HSDAVFTDNYTRLRKQVAAKAibYLQSIKNKRYGGPSSGAPPPS |
| SEQ ID NO: 154 | |
| P171 | C6-HSDAVFTDNYTRLKKQVAAKKYLQSIKNKRYGGPSSGAPPPS |
| SEQ ID NO: 155 | |
| P172 | C6-HSDAVFTDNYTRLQKQVAAKKYLQSIKNKRYGGPSSGAPPPS |
| SEQ ID NO: 156 | |
| P173 | C6-HSDAVFTDNYTRLAKQVAAKKYLQSIKNKRYGGPSSGAPPPS |
| SEQ ID NO: 157 | |
| P174 | C6-HSDAVFTDNYTRLLKQVAAKKYLQSIKNKRYGGPSSGAPPPS |
| SEQ ID NO: 158 | |
| P175 | C6-HSDAVFTDNYTRLFKQVAAKKYLQSIKNKRYGGPSSGAPPPS |
| SEQ ID NO: 159 | |
| P176 | C6-HSDAVFTDNYTRLRRQVAAKKYLQSIKNKRYGGPSSGAPPPS |
| SEQ ID NO: 160 | |
| P177 | C6-HSDAVFTDNYTRLRQQVAAKKYLQSIKNKRYGGPSSGAPPPS |
| SEQ ID NO: 161 | |
| P179 | C6-HSDAVFTDNYTRLRLQVAAKKYLQSIKNKRYGGPSSGAPPPS |
| SEQ ID NO: 162 | |
| P180 | C6-HSDAVFTDNYTRLRFQVAAKKYLQSIKNKRYGGPSSGAPPPS |
| SEQ ID NO: 163 | |
| P181 | C6-HSDAVFTDNYTRLRKQVAAAKYLQSIKNKRYGGPSSGAPPPS |
| SEQ ID NO: 164 | |
| P182 | C6-HSDAVFTDNYTRLRKQVAAKRYLQSIKNKRYGGPSSGAPPPS |
| SEQ ID NO: 165 | |
| P183 | C6-HSDAVFTDNYTRLRKQVAAKQYLQSIKNKRYGGPSSGAPPPS |
| SEQ ID NO: 166 | |
| P184 | C6-HSDAVFTDNYTRLRKQVAAKAYLQSIKNKRYGGPSSGAPPPS |
| SEQ ID NO: 167 | |
| P185 | C6-HSDAVFTDNYTRLRKQVAAKLYLQSIKNKRYGGPSSGAPPPS |
| SEQ ID NO: 168 | |
| P186 | C6-HSDAVFTDNYTRLRKQVAAKFYLQSIKNKRYGGPSSGAPPPS |
| SEQ ID NO: 169 | |
| P187 | C6-HSDAVFTDNYTRLRKQVAAKKYLQAibIKNKRYGGPSSGAPPPS |
| SEQ ID NO: 170 | |
| P188 | C6-HSDAVFTDNYTRLRKQVAAKKYLQSAibKNKRYGGPSSGAPPPS |
| SEQ ID NO: 171 | |
| P189 | C6-HSDAVFTDNYTRLRKQAibAAKKYLQSIKNKRYGGPSSGAPPPS |
| SEQ ID NO: 172 | |
| P191 | C6-HHSDAVFTDNYTRLLAKLALQKYLQSIKNKRYGGPSSGAPPPS |
| SEQ ID NO: 173 | |
| P192 | C6-HSDAVFTDQYTRLLAKLALQKYLQSIKQKRYGGPSSGAPPPS |
| SEQ ID NO: 174 | |
| P193 | C6- |
| SEQ ID NO: 175 | HSDAVFTDNYTRLRK(Ac)QVAAK(Ac)KYLQSIKNKRYGGPSSGAPPPS |
| P194 | C6- |
| SEQ ID NO: 176 | HSDAVFTDNYTRLRK(Ac)QVAAKK(Ac)YLQSIKNKRYGGPSSGAPPPS |
| P195 | C6-HSDAVFTDNYTRLLAQLALQKYLQSIKNKRYGGPSSGAPPPS |
| SEQ ID NO: 177 | |
| P196 | C6-HSDAVFTDNYTRLLAKVALQKYLQSIKNKRYGGPSSGAPPPS |
| SEQ ID NO: 178 | |
| P197 | C6-HSDAVFTDNYTRLLAKLAAQKYLQSIKNKRYGGPSSGAPPPS |
| SEQ ID NO: 179 | |
| P198 | C6-HSDAVFTDNYTRLLAKLALQKYLQSIKNKGGPSSGAPPPC |
| SEQ ID NO: 180 | |
| P199 | Met(O)-HSDAVFTDNYTRLRKQVAAKKYLQSIKNKRYGGPSSGAPPPS |
| SEQ ID NO: 181 | |
| P203 | C6-HSDAVFTDNYTRLRKQVAAKKYLQSIKNKRFGGPSSGAPPPS |
| SEQ ID NO: 182 | |
| P205 | HS(CH₂)₂CO- |
| SEQ ID NO: 183 | HSDAVFTDNYTRLLAKLALQKYLQSIKNKRYGGPSSGAPPPS |
| P206 | HS(CH₂)₂CO- |
| SEQ ID NO: 184 | HSDAVFTDNYTRLRKQVAAKKYLQSIKNKRYGGPSSGAPPPS |
| P207 | C6-HSDdAVFTDNYTRLRKQVAAKKYLQSIKNKRYGGPSSGAPPPS |
| SEQ ID NO: 185 | |
| P208 | C6-HSDNMeAVFTDNYTRLRKQVAAKKYLQSIKNKRYGGPSSGAPPPS |
| SEQ ID NO: 186 | |
| P209 | C6-HSDAibVFTDNYTRLRKQVAAKKYLQSIKNKRYGGPSSGAPPPS |
| SEQ ID NO: 187 | |
| P210 | C6-HSDAdVFTDNYTRLRKQVAAKKYLQSIKNKRYGGPSSGAPPPS |
| SEQ ID NO: 188 | |
| P211 | C6-HSDANMeVFTDNYTRLRKQVAAKKYLQSIKNKRYGGPSSGAPPPS |
| SEQ ID NO: 189 | |
| P212 | C6-HSDAAibFTDNYTRLRKQVAAKKYLQSIKNKRYGGPSSGAPPPS |
| SEQ ID NO: 190 | |
| P213 | C6-HSDAVFTDNYTRLRKQVAAKRYLQSIRNGGPSSGAPPPS |
| SEQ ID NO: 191 | |
| P214 | C6-HSDAVFTDNYTRLRKQVAARRYLQSIRNGGPSSGAPPPS |
| SEQ ID NO: 192 | |
| P215 | C6-HSDAVFTDNYTRLRRQVAAKRYLQSIRNGGPSSGAPPPS |
| SEQ ID NO: 193 | |
| P216 | C6-HSDAVFTDNYTRLRRQVAARKYLQSIRNGGPSSGAPPPS |
| SEQ ID NO: 194 | |
| P220 | C6-HSDAVFTDQYTRLRRQVAARKYLQSIRQGGPSSGAPPPS |
| SEQ ID NO: 195 | |
| P221 | C6-HSDIVFTDNYTRLRKQVAAKKYLQSIKNKRYGGPSSGAPPPS |
| SEQ ID NO: 196 | |
| P222 | C6-HGEGTFTDNYTRLRKQVAAKKYLQSIKNKRYGGPSSGAPPPS |
| SEQ ID NO: 197 | |
| P223 | C6-HSDLVFTDNYTRLRKQVAAKKYLQSIKNKRYGGPSSGAPPPS |
| SEQ ID NO: 198 | |
| P224 | C6-HSEAVFTDNYTRLRKQVAAKKYLQSIKNKRYGGPSSGAPPPS |
| SEQ ID NO: 199 | |
| P228 | C6-HSDAVFTDNYTRLRKQVAAKKAibLQSIKNKRYGGPSSGAPPPS |
| SEQ ID NO: 200 | |
| P229 | C6-HSDAVFTDNYTRLRKQVAAKKALQSIKNKRYGGPSSGAPPPS |
| SEQ ID NO: 201 | |
| P230 | C6-HSDAVFTDNYTRLRKQVAAKKYWQSIKNKRYGGPSSGAPPPS |
| SEQ ID NO: 202 | |
| P231 | C6-HSDAVFTDNYTRLRKQVAAKKYFQSIKNKRYGGPSSGAPPPS |
| SEQ ID NO: 203 | |
| P232 | C6-HSDAVFTDNYTRLRKQVAAKKYTQSIKNKRYGGPSSGAPPPS |
| SEQ ID NO: 204 | |
| P233 | C6-HSDAVFTDNYTRLRKQVAAKKYIQSIKNKRYGGPSSGAPPPS |
| SEQ ID NO: 205 | |
| P234 | C6-HSDAVFTDNYTRLRKQVAAKKYVQSIKNKRYGGPSSGAPPPS |
| SEQ ID NO: 206 | |
| P235 | C6-HSDAVFTDNYTRLRKQVAAKKYAQSIKNKRYGGPSSGAPPPS |
| SEQ ID NO: 207 | |
| P236 | C6-HSDAVFTDNYTRLRKQVAAKKYAibQSIKNKRYGGPSSGAPPPS |
| SEQ ID NO: 208 | |
| P240 | C6-HSDAVFTDNYTRLAibKQLAAAibKYLQSIKNKRYGGPSSGAPPPS |
| SEQ ID NO: 209 | |
| P241 | C6-HSDAVFTDNYTRLAibKQLAAKAibYLQSIKNKRYGGPSSGAPPPS |
| SEQ ID NO: 210 | |
| P242 | C6-HSDAVFTDNYTRLAibKQVAAAibKYLQSIKNKRYGGPSSGAPPPS |
| SEQ ID NO: 211 | |
| P243 | C6-HSDAVFTDNYTRLAibKQVAAQKYLQSIKNKRYGGPSSGAPPPS |
| SEQ ID NO: 212 | |
| P244 | C6-HSDAVFTDNYTRLAibKQVAAKAibYLQSIKNKRYGGPSSGAPPPS |
| SEQ ID NO: 213 | |
| P249 | C6-HSDAVFTDNYTRLAibKQVAAKQYLQSIKRYGGPSSGAPPPS |
| SEQ ID NO: 214 | |
| P250 | C6-HSDAVFTDNYTRLQKQVAAAibKYLQSIKNKRYGGPSSGAPPPS |
| SEQ ID NO: 215 | |
| P251 | C6-HSDAVFTDNYTRLQKQVAAKAibYLQSIKNKRYGGPSSGAPPPS |
| SEQ ID NO: 216 | |
| P252 | C6-HSDAVFTDNYTRLQKQVAAQKYLQSIKNKRYGGPSSGAPPPS |
| SEQ ID NO: 217 | |
| P253 | C6-HSDAVFTDNYTRLQKQVAAKQYLQSIKNKRYGGPSSGAPPPS |
| SEQ ID NO: 218 | |
| P258 | C6-HSDAVFTDNYTRLQAibQVAAKKYLQSIKNKRYGGPSSGAPPPS |
| SEQ ID NO: 219 | |
| P259 | C6-HSDAVFTDNYTRLAibKQVAALKYLQSIKNKRYGGPSSGAPPPS |
| SEQ ID NO: 220 | |
| P260 | C6-HSDAVFTDNYTRLAibKQVAAAKYLQSIKNKRYGGPSSGAPPPS |
| SEQ ID NO: 221 | |
| P261 | C6-HSDAVFTDNYTRLRAibQVAAAibKYLQSIKNKRYGGPSSGAPPPS |
| SEQ ID NO: 222 | |
| P262 | C6-HSDAVFTDNYTRLRAibQVAAVKYLQSIKNKRYGGPSSGAPPPS |
| SEQ ID NO: 223 | |
| P263 | C6-HSDAVFTDNYTRLRAibQVAAAKYLQSIKNKRYGGPSSGAPPPS |
| SEQ ID NO: 224 | |
| P264 | C6-HSDAVFTDNYTRLRAibQVAAKAibYLQSIKNKRYGGPSSGAPPPS |
| SEQ ID NO: 225 | |
| P265 | C6-HSDAVFTDNYTRLRAibQVAALKYLQSIKNKRYGGPSSGAPPPS |
| SEQ ID NO: 226 | |
| P269 | C6-HSDAVFTDNYTRLAibKQVAAVKYLQSIKNKRYGGPSSGAPPPS |
| SEQ ID NO: 227 | |
| P270 | C6-HSDAVFTDNYTRLRKQVAAKKYLQSIKQKGGPSSGAPPPS |
| SEQ ID NO: 228 | |
| P271 | C6-HSDAVFTDNYTRLRKQVAAKKYLQSIKNGRPSSGAPPPS |
| SEQ ID NO: 229 | |
| P275 | C6-HSDAVFTDNYTRLRKQVAGKKYLQSIKNKRYGGPSSGAPPPS |
| SEQ ID NO: 230 | |
| P282 | C6-HSDAVFTDNYTRLAibKQVAAAibKYLQSIKNKRYGGPSSGAPPPC- |
| SEQ ID NO: 231 | NH₂ |
| P284 | C6-HSDAVFTDNYTRLRAibQLAAKAibYLQSIKNKRYGGPSSGAPPPS |
| SEQ ID NO: 232 | |
| P285 | C6-HSDAVFTDNYTRLRAibQVAAKAibYLQSIKNKRYGGPSSGAPPPC- |
| SEQ ID NO: 233 | NH₂ |
| P289 | C6-HSDALFTDNYTRLRKQVAAKKYLQSIKNKRYGGPSSGAPPPS |
| SEQ ID NO: 234 | |
| P290 | C6-HSDAVFTDNYTRLRKQLAAKKYLQSIKNKRYGGPSSGAPPPS |
| SEQ ID NO: 235 | |
| P291 | C6-HSDAVFTDNYTRLRAibQLAAAibKYLQSIKNKRYGGPSSGAPPPS |
| SEQ ID NO: 236 | |
| P292 | C6-HSDAVFTDNYTRLRAibQLAAAibKYLQSIKNKGGPSSGAPPPS |
| SEQ ID NO: 237 | |
| P293 | C6-HSDAVFTDNYTRLAibKQVAAAibKYLQSIKQKGGPSSGAPPPS |
| SEQ ID NO: 238 | |
| P294 | C6-HSDAVFTDNYTRLRAibQVAAAibKYLQSIKQKGGPSSGAPPPS |
| SEQ ID NO: 239 | |
| P295 | C6-HSDAVFTDNYTRLRAibQVAAKAibYLQSIKQKGGPSSGAPPPS |
| SEQ ID NO: 240 | |
| P296 | C6-HSDAVFTDNYTRLAibKQVAAAibKYLQSIKQGGPSSGAPPPS |
| SEQ ID NO: 241 | |
| P297 | C6-HSDAVFTDNYTRLRAibQVAAAibKYLQSIKQGGPSSGAPPPS |
| SEQ ID NO: 242 | |
| P298 | C6-HSDAVFTDNYTRLRAibQVAAKAibYLQSIKQGGPSSGAPPPS |
| SEQ ID NO: 243 | |
| P299 | C6-HSDAVFTDNYTRLRAibQVAAAibKYLQSIKNKRYGGPSSGAPPPC- |
| SEQ ID NO: 244 | NH₂ |
| P301 | C6-HSDAVFTDNYTRLAAibQVAAAibKYLQSIKNKRYGGPSSGAPPPS |
| SEQ ID NO: 245 | |
| P302 | C6-HSDAVFTDNYTRLQAibQVAAAibKYLQSIKNKRYGGPSSGAPPPS |
| SEQ ID NO: 246 | |
| P305 | C6-HSDAVFTDNYTRLhRKQVAAKKYLQSIKNKRYGGPSSGAPPPS |
| SEQ ID NO: 247 | |
| P307 | C6-HSDAVFTDNYTRLROrnQVAAKKYLQSIKNKRYGGPSSGAPPPS |
| SEQ ID NO: 248 | |
| P308 | C6-HSDAVFTDNYTRLhROrnQVAAKKYLQSIKNKRYGGPSSGAPPPS |
| SEQ ID NO: 249 | |
| P314 | C6-HSEAVFTENYTRLRAibQVAAAibKYLQSIKNKRYGGPSSGAPPPS |
| SEQ ID NO: 250 | |
| P315 | C6-HSDAVFTDQYTRLRAibQVAAAibKYLQSIKQKRYGGPSSGAPPPS |
| SEQ ID NO: 251 | |
| P316 | C6-HSDAVFTDNYTRLhRAibQVAAAibKYLQSIKNKRYGGPSSGAPPPS |
| SEQ ID NO: 252 | |
| P317 | C6-HSDAVFTDNYTRLLAibQVAAAibKYLQSIKNKRYGGPSSGAPPPS |
| SEQ ID NO: 253 | |
| P318 | C6-HSDAVFTDNYTRLKAibQVAAAibKYLQSIKNKRYGGPSSGAPPPS |
| SEQ ID NO: 254 | |
| P319 | C6-HSDAVFTDNYTRLOrnAibQVAAAibKYLQSIKNKRYGGPSSGAPPPS |
| SEQ ID NO: 255 | |
| P320 | C6-HSDAVFTDNYTRLCitAibQVAAAibKYLQSIKNKRYGGPSSGAPPPS |
| SEQ ID NO: 256 | |
| P321 | C6-HSDAVFTDNYTRLRAibKVAAAibKYLQSIKNKRYGGPSSGAPPPS |
| SEQ ID NO: 257 | |
| P322 | C6-HSDAVFTDNYTRLRAibQIAAAibKYLQSIKNKRYGGPSSGAPPPS |
| SEQ ID NO: 258 | |
| P323 | C6-HSDAVFTDNYTRLRAibQKAAAibKYLQSIKNKRYGGPSSGAPPPS |
| SEQ ID NO: 259 | |
| P324 | C6-HSDAVFTDNYTRLRAibQAAAAibKYLQSIKNKRYGGPSSGAPPPS |
| SEQ ID NO: 260 | |
| P325 | C6-HSDAVFTDNYTRLRAibQNleAAAibKYLQSIKNKRYGGPSSGAPPPS |
| SEQ ID NO: 261 | |
| P326 | C6- |
| SEQ ID NO: 262 | HSDAVFTDNYTOrnLRAibQVAAAibKYLQSIOrnNOrnGGPSSGAPPPS |
| P327 | C6- |
| SEQ ID NO: 263 | HSDAVFTDNYTOrnLRAibQVAAAibKYLQSIOrnNOrnGGPSSGAPPPC- |
| | NH₂ |
| P329 | C6-HSDAVFTDNYThRLRAibQVAAAibKYLQSIhRNhRGGPSSGAPPPS |
| SEQ ID NO: 264 | |
| P330 | C6-HSDAVFTDNYThRLRAibQVAAAibKYLQSIhRNhRGGPSSGAPPPC- |
| SEQ ID NO: 265 | NH₂ |
| P332 | HSDAVFTDNYThRLRAibQVAAAibKYLQSIhRhRGGPSSGAPPPS |
| SEQ ID NO: 266 | |
| P333 | C6-HSDAVFTDNYThRLRAibQVAAAibKYLQSIhRhRGGPSSGAPPPC- |
| SEQ ID NO: 267 | NH₂ |
| P335 | C6-HSDAVFTDNYTOrnLRAibQVAAAibKYLQSIhRhRGGPSSGAPPPS |
| SEQ ID NO: 268 | |
| P336 | C6-HSDAVFTDNYTOrnLRAibQVAAAibKYLQSIhRhRGGPSSGAPPPC- |
| SEQ ID NO: 269 | NH₂ |
| P338 | C6- |
| SEQ ID NO: 270 | HSDAVFTDNYThRLRAibQVAAAibKYLQSIhRNhRhRYGGPSSGAPPPS |
| P339 | C6- |
| SEQ ID NO: 271 | HSDAVFTDNYThRLRAibQVAAAibKYLQSIhRNhRhRYGGPSSGAPPPC- |
| | NH₂ |
| P341 | C6-HSDAVFTDNYTRLRAibQVAAAibAYLQSIKNKRYGGPSSGAPPPS |
| SEQ ID NO: 272 | |
| P342 | C6-HSDAVFTDNYTRLRAibQVAAAibOrnYLQSIKNKRYGGPSSGAPPPS |
| SEQ ID NO: 273 | |
| P344 | C6-HSDAVFTDNYTOrnLRAibQVAAAibKYLQSIKNKRYGGPSSGAPPPS |
| SEQ ID NO: 274 | |
| P345 | C6-HSDAVFTDNYTAibLRAibQVAAAibKYLQSIKNKRYGGPSSGAPPPS |
| SEQ ID NO: 275 | |
| P346 | C6-HSDAVFTDNYTRAibRAibQVAAAibKYLQSIKNKRYGGPSSGAPPPS |
| SEQ ID NO: 276 | |
| P349 | C6-HSDAVFTDNYTRLRAibQVAAAibKYLQSIKAibKGGPSSGAPPPS |
| SEQ ID NO: 277 | |
| P350 | C6-HSDAVFTDNYTRLRAibQVAAAibKYLQSIKPKGGPSSGAPPPS |
| SEQ ID NO: 278 | |
| P351 | C6-HSDAVFTDNYTRLRAibQVAAAibKYLQSIKKGGPSSGAPPPS |
| SEQ ID NO: 279 | |
| P352 | C6-HSDAVFTDNYTRLRAibQVAAAibKYLQSIOrnOrnGGPSSGAPPPS |
| SEQ ID NO: 280 | |
| P353 | C6-HSDAVFTDNYTRLRAibQVAAAibKYLQSIdKdKGGPSSGAPPPS |
| SEQ ID NO: 281 | |
| P354 | C6-HSDAVFTDNYTRLRAibQVAAAibKYLQSIKhRGGPSSGAPPPS |
| SEQ ID NO: 282 | |
| P355 | C6-HSDAVFTDNYTRLRAibQVAAAibKYLQSIKAibGGPSSGAPPPS |
| SEQ ID NO: 283 | |
| P356 | C6-HSDAVFTDNYTRLRAibQVAAAibKYLQSIOrnQOrnGGPSSGAPPPS |
| SEQ ID NO: 284 | |
| P364 | C6-HSDAVFTDNYTOrnLRAibQLAAAibKYLQSIOrnNOrnGGPSSGAPPPS |
| SEQ ID NO: 285 | |
| P365 | C6-HSDAVFTDNYTOrnLRAibQIAAAibKYLQSIOrnNOrnGGPSSGAPPPS |
| SEQ ID NO: 286 | |
| P366 | C6-HSDAVFTDNYTALRAibQVAAAibKYLQSIOrnNOrnGGPSSGAPPPS |
| SEQ ID NO: 287 | |

More preferred VPAC2 peptide receptor agonists according to the second aspect of the present invention comprise an amino acid sequence selected from:

| Agonist | |
|---|---|
| # | Sequence |
| P18 | HSDAVFTDNYTRLRKQVAAhRKYLQSIKNKRYGGPSSGAPPPS |
| P20 | HSDAVFTDNYTRLRKQVAARKYLQSIKNKRYGGPSSGAPPPS |
| P21 | HSDAVFTDNYTRLRKQVAASKYLQSIKNKRYGGPSSGAPPPS |
| P22 | HSDAVFTDNYTRLRKQVAAKKYLQSIhRNKRYGGPSSGAPPPS |
| P23 | HSDAVFTDNYTRLRKQVAAKKYLQSIRNKRYGGPSSGAPPPS |
| P31 | C6-HSDAVFTDNYTRLRKQVAAKKYLQSIKNKRYGGPSSGAPPPS |
| P33 | Ac-HSDAVFTDNYTRLRKQVAAKKYLQSIKNKRYGGPSSGAPPPS |
| P34 | HSDAVFTDNYTRLRKQVAAKKYLQSIKNKRF |
| P44 | HVDAFTKNYTRLRKQVAAKKYLQSIKNKRYGGPSSGAPPPS |
| P47 | HdADAVFTDNYTRLRKQVAAKKYLQSIKNKRYGGPSSGAPPPS |
| P48 | HdSDAVFTDNYTRLRKQVAAKKYLQSIKNKRYGGPSSGAPPPS |
| P61 | HSDALFTDNYTRLRKQVAAKKYLQSIKNKRYGGPSSGAPPPS |
| P72 | HSDAVFTDNYTRLRRQVAAKKYLQSIKNKRYGGPSSGAPPPS |
| P89 | HSDAVFTDNYTRLLAKVAAKKYLQSIKNKRYGGPSSGAPPPS |
| P98 | C6-HSDAVFTDNYTRLRRQVAARRYLQSIRNRRYGGPSSGAPPPS |
| P99 | C6-HVDAVFTDNYTRLRKQVAAKKYLQSIKNKRYGGPSSGAPPPS |
| P100 | M-HSDAVFTDQYTRLRKQVAAKKYLQSIKQKRYGGPSSGAPPPS |
| P101 | C6-HdADAVFTDNYTRLRKQVAAKKYLQSIKNKRYGGPSSGAPPPS |
| P103 | C6-HSDAVFTDNYTKLKKQVAAKKYLQSIKNKKYGGPSSGAPPPS |
| P104 | M-HSDAVFTDNYTRLRKQVAAKKYLQSIKNKRYGGPSSGAPPPS |
| P106 | C6-HSDAVFTDNYTRLRKQVAAKKYLQSFKNKRYGGPSSGAPPPS |
| P107 | C6-HSDAVFTDNYTRLRKQVAAKKYLQSLKNKRYGGPSSGAPPPS |
| P108 | C6-HSDAVFTDNYTRLRKQVAAKKYLQSTKNKRYGGPSSGAPPPS |
| P109 | C6-HSDAVFTDNYTRLRKQVAAKKYLQSVKNKRYGGPSSGAPPPS |
| P110 | C6-HSDAVFTDNYTRLRKQVAAKKYLQSWKNKRYGGPSSGAPPPS |
| P111 | C6-HSDAVFTDNYTRLRKQVAAKKYLQSYKNKRYGGPSSGAPPPS |
| P112 | C6-HSDAVFTDNYTRLRKQVAAKKYLQFIKNKRYGGPSSGAPPPS |
| P113 | C6-HSDAVFTDNYTRLRKQVAAKKYLQIIKNKRYGGPSSGAPPPS |
| P114 | C6-HSDAVFTDNYTRLRKQVAAKKYLQLIKNKRYGGPSSGAPPPS |
| P115 | C6-HSDAVFTDNYTRLRKQVAAKKYLQTIKNKRYGGPSSGAPPPS |
| P116 | C6-HSDAVFTDNYTRLRKQVAAKKYLQVIKNKRYGGPSSGAPPPS |
| P117 | C6-HSDAVFTDNYTRLRKQVAAKKYLQWIKNKRYGGPSSGAPPPS |
| P119 | C6-HSDAVFTDNYTRLLAKLALQKYLQSIKNKRYGGPSSGAPPPS |
| P120 | C6-HSDAVFTDNYTRLRKQVAAKKYLQSIKNKRYGGPSSGAPPPC |
| P121 | C6-HSDAVFTDNYTRLRKQVAAKKYLQSIKNKKGGPSSGAPPPS |
| P122 | C6-HSDAVFTDNYTRLRKQVAAKKYLQSIKNSRGGPSSGAPPPS |
| P123 | C6-HSDAVFTDNYTRLRKQVAAKKYLQSIKNKRGGPSSGAPPPS |
| P124 | C6-HSDAVFTDNYTRLRKQVAAKKYLQQIKNKRYGGPSSGAPPPS |
| P125 | C6-HSDAVFTDNYTRLRKQVAAKKYLQNIKNKRYGGPSSGAPPPS |
| P127 | C6-HSDAVFTDNYTRLRKQVAAKKYLQYIKNKRYGGPSSGAPPPS |
| P129 | C6-HSDAVFTDNYTRLRKQVAAKKWLQSIKNKRYGGPSSGAPPPS |
| P130 | C6-HSDAVFTDNYTRLRKQVAAKKFLQSIKNKRYGGPSSGAPPPS |
| P132 | C6-HSDAVFTDNYTRLRKQVAAKKLLQSIKNKRYGGPSSGAPPPS |
| P133 | C6-HSDAVFTDNYTRLRKQVAAKKILQSIKNKRYGGPSSGAPPPS |
| P134 | C6-HSDAVFTDNYTRLRKQVAAKKVLQSIKNKRYGGPSSGAPPPS |
| P135 | C6-HSDAVFTDNYTRLLAKVAAKKYLQSIKNKRYGGPSSGAPPPS |
| P138 | C6-HSDAVFTDNYTRLRAQVAAQKYLQSIKNKRYGGPSSGAPPPS |
| P139 | C6-HSDAVFTDNYTRLRAQVAAKKYLQSIKNKRYGGPSSGAPPPS |
| P142 | C6-HSDAVFTDNYTRLRAQLAAQKYLQSIKNKRYGGPSSGAPPPS |
| P143 | C6-HSDAVFTDNYTRLRKQMAAQKYLNQLKKGGPSSGAPPPS |
| P144 | C6-HSDAVFTDNYTRLRKQVAAQKYLNQLKKGGPSSGAPPPS |
| P146 | C6-HSDAVFTDNYTRLRKQVAAVKYLQSIKNKRYGGPSSGAPPPS |
| P147 | C6-HSDAVFTDNYTRLRKQVAAYKYLQSIKNKRYGGPSSGAPPPS |
| P148 | C6-HSDAVFTDNYTRLRKQVAAFKYLQSIKNKRYGGPSSGIWPPS |
| P149 | C6-HSDAVFTDNYTRLRKQVAAIKYLQSIKNKRYGGPSSGAPPPS |
| P150 | C6-HSDAVFTDNYTRLRKQVAAQKYLQSIKNKRYGGPSSGAPPPS |
| P151 | C6-HSDAVFTDNYTRLRKQVAALKYLQSIKNKRYGGPSSGAPPPS |
| P152 | C6-HSDAVFTDNYTRLRKQVAATKYLQSIKNKRYGGPSSGAPPPS |
| P153 | C6-HSDAVFTDNYTRLRKQVAAWKYLQSIKNKRYGGPSSGAPPPS |
| P154 | C6-HSDAVFTDNYTRLRKQVAAKKYLQSIKNGGPSSGAPPPS |
| P155 | C6-HSDAVFTDNYTRLRKQVALKKYLQSIKNKRYGGPSSGAPPPS |
| P158 | C6-HSDAVFTANYTRLRKQVAAKKYLQSIKNKRYGGPSSGAPPPS |
| P159 | C6-HSDAVFTENYTRLRKQVAAKKYLQSIKNKRYGGPSSGAPPPS |
| P164 | C6-HSDAVFTDNYTRLLAKLALQKYLQSIKNKRYGGPSSGAPPPC |
| P165 | C6-HSDAVFTEEYTRLQKQVAAKQYLQSIKNKRYGGPSSGAPPPS |
| P166 | C6-HAibDAVFTDNYTRLRKQVAAKKYLQSIKNKRYGGPSSGAPPPS |
| P167 | C6-HSDAVFTDNYTRLAibKQVAAKKYLQSIKNKRYGGPSSGAPPPS |
| P168 | C6-HSDAVFTDNYTRLRAibQVAAKKYLQSIKNKRYGGPSSGAPPPS |
| P169 | C6-HSDAVFTDNYTRLRKQVAAAibKYLQSIKNKRYGGPSSGAPPPS |
| P170 | C6-HSDAVFTDNYTRLRKQVAAKAibYLQSIKNKRYGGPSSGAPPPS |
| P171 | C6-HSDAVFTDNYTRLKKQVAAKKYLQSIKNKRYGGPSSGAPPPS |
| P172 | C6-HSDAVFTDNYTRLQKQVAAKKYLQSIKNKRYGGPSSGAPPPS |
| P173 | C6-HSDAVFTDNYTRLAKQVAAKKYLQSIKNKRYGGPSSGAPPPS |
| P175 | C6-HSDAVFTDNYTRLFKQVAAKKYLQSIKNKRYGGPSSGAPPPS |
| P176 | C6-HSDAVFTDNYTRLRRQVAAKKYLQSIKNKRYGGPSSGAPPPS |
| P177 | C6-HSDAVFTDNYTRLRQQVAAKKYLQSIKNKRYGGPSSGAPPPS |
| P179 | C6-HSDAVFTDNYTRLRLQVAAKKYLQSIKNKRYGGPSSGAPPPS |
| P180 | C6-HSDAVFTDNYTRLRFQVAAKKYLQSIKNKRYGGPSSGAPPPS |
| P181 | C6-HSDAVFTDNYTRLRKQVAAAKYLQSIKNKRYGGPSSGAPPPS |
| P182 | C6-HSDAVFTDNYTRLRKQVAAKRYLQSIKNKRYGGPSSGAPPPS |
| P183 | C6-HSDAVFTDNYTRLRKQVAAKQYLQSIKNKRYGGPSSGAPPPS |
| P184 | C6-HSDAVFTDNYTRLRKQVAAKAYLQSIKNKRYGGPSSGAPPPS |
| P185 | C6-HSDAVFTDNYTRLRKQVAAKLYLQSIKNKRYGGPSSGAPPPS |
| P186 | C6-HSDAVFTDNYTRLRKQVAAKFYLQSIKNKRYGGPSSGAPPPS |
| P187 | C6-HSDAVFTDNYTRLRKQVAAKKYLQAibIKNKRYGGPSSGAPPPS |
| P188 | C6-HSDAVFTDNYTRLRKQVAAKKYLQSAibKNKRYGGPSSGAPPPS |
| P192 | C6-HSDAVFTDQYTRLLAKLALQKYLQSIKQKRYGGPSSGAPPPS |
| P193 | C6-HSDAVFTDNYTRLRK(Ac)QVAAK(Ac)KYLQSIKNKRYGGPSSGAPPPS |
| P194 | C6-HSDAVFTDNYTRLRK(Ac)QVAAKK(Ac)YLQSIKNKRYGGPSSGAPPPS |
| P195 | C6-HSDAVFTDNYTRLLAQLALQKYLQSIKNKRYGGPSSGAPPPS |
| P196 | C6-HSDAVFTDNYTRLLAKVALQKYLQSIKNKRYGGPSSGAPPPS |
| P197 | C6-HSDAVFTDNYTRLLAKLAAQKYLQSIKNKRYGGPSSGAPPPS |
| P207 | C6-HSDdAVFTDNYTRLRKQVAAKKYLQSIKNKRYGGPSSGAPPPS |
| P209 | C6-HSDAibVFTDNYTRLRKQVAAKKYLQSIKNKRYGGPSSGAPPPS |
| P210 | C6-HSDAdVFTDNYTRLRKQVAAKKYLQSIKNKRYGGPSSGAPPPS |
| P212 | C6-HSDAAibFTDNYTRLRKQVAAKKYLQSIKNKRYGGPSSGAPPPS |
| P213 | C6-HSDAVFTDNYTRLRKQVAAKRYLQSIRNGGPSSGAPPPS |
| P214 | C6-HSDAVFTDNYTRLRKQVAARRYLQSIRNGGPSSGAPPPS |
| P215 | C6-HSDAVFTDNYTRLRRQVAAKRYLQSIRNGGPSSGAPPPS |
| P216 | C6-HSDAVFTDNYTRLRRQVAARKYLQSIRNGGPSSGAPPPS |
| P240 | C6-HSDAVFTDNYTRLAibKQLAAAibKYLQSIKNKRYGGPSSGAPPPS |
| P241 | C6-HSDAVFTDNYTRLAibKQLAAKAibYLQSIKNKRYGGPSSGAPPPS |
| P242 | C6-HSDAVFTDNYTRLAibKQVAAAibKYLQSIKNKRYGGPSSGAPPPS |
| P243 | C6-HSDAVFTDNYTRLAibKQVAAQKYLQSIKNKRYGGPSSGAPPPS |
| P244 | C6-HSDAVFTDNYTRLAibKQVAAKAibYLQSIKNKRYGGPSSGAPPPS |
| P249 | C6-HSDAVFTDNYTRLAibKQVAAKQYLQSIKNKRYGGPSSGAPPPS |
| P250 | C6-HSDAVFTDNYTRLQKQVAAAibKYLQSIKNKRYGGPSSGAPPPS |
| P251 | C6-HSDAVFTDNYTRLQKQVAAKAibYLQSIKNKRYGGPSSGAPPPS |
| P258 | C6-HSDAVFTDNYTRLQAibQVAAKKYLQSIKNKRYGGPSSGAPPPS |
| P259 | C6-HSDAVFTDNYTRLAibKQVAALKYLQSIKNKRYGGPSSGAPPPS |
| P260 | C6-HSDAVFTDNYTRLAibKQVAAAKYLQSIKNKRYGGPSSGAPPPS |
| P261 | C6-HSDAVFTDNYTRLRAibQVAAAibKYLQSIKNKRYGGPSSGAPPPS |
| P262 | C6-HSDAVFTDNYTRLRAibQVAAVKYLQSIKNKRYGGPSSGAPPPS |
| P263 | C6-HSDAVFTDNYTRLRAibQVAAAKYLQSIKNKRYGGPSSGAPPPS |
| P264 | C6-HSDAVFTDNYTRLRAibQVAAKAibYLQSIKNKRYGGPSSGAPPPS |
| P265 | C6-HSDAVFTDNYTRLRAibQVAALKYLQSIKNKRYGGPSSGAPPPS |
| P269 | C6-HSDAVFTDNYTRLAibKQVAAVKYLQSIKNKRYGGPSSGAPPPS |
| P284 | C6-HSDAVFTDNYTRLRAibQLAAKAibYLQSIKNKRYGGPSSGAPPPS |
| P291 | C6-HSDAVFTDNYTRLRAibQLAAAibKYLQSIKNKRYGGPSSGAPPPS |
| P292 | C6-HSDAVFTDNYTRLRAibQLAAAibKYLQSIKNKGGPSSGAPPPS |
| P293 | C6-HSDAVFTDNYTRLAibKQVAAAibKYLQSIKQKGGPSSGAPPPS |
| P294 | C6-HSDAVFTDNYTRLRAibQVAAAibKYLQSIKQKGGPSSGAPPPS |
| P295 | C6-HSDAVFTDNYTRLRAibQVAAKAibYLQSIKQKGGPSSGAPPPS |
| P296 | C6-HSDAVFTDNYTRLAibKQVAAAibKYLQSIKQGGPSSGAPPPS |
| P297 | C6-HSDAVFTDNYTRLRAibQVAAAibKYLQSIKQGGPSSGAPPPS |
| P298 | C6-HSDAVFTDNYTRLRAibQVAAKAibYLQSIKQGGPSSGAPPPS |
| P301 | C6-HSDAVFTDNYTRLAAibQVAAAibKYLQSIKNKRYGGPSSGAPPPS |
| P302 | C6-HSDAVFTDNYTRLQAibQVAAAibKYLQSIKNKRYGGPSSGAPPPS |
| P305 | C6-HSDAVFTDNYTRLhRKQVAAKKYLQSIKNKRYGGPSSGAPPPS |
| P307 | C6-HSDAVFTDNYTRLROrnQVAAKKYLQSIKNKRYGGPSSGAPPPS |
| P314 | C6-HSEAVFTENYTRLRAibQVAAAibKYLQSIKNKRYGGPSSGAPPPS |
| P317 | C6-HSDAVFTDNYTRLLAibQVAAAibKYLQSIKNKRYGGPSSGAPPPS |
| P318 | C6-HSDAVFTDNYTRLKAibQVAAAibKYLQSIKNKRYGGPSSGAPPPS |
| P319 | C6-HSDAVFTDNYTRLOrnAibQVAAAibKYLQSIKNKRYGGPSSGAPPPS |
| P321 | C6-HSDAVFTDNYTRLRAibKVAAAibKYLQSIKNKRYGGPSSGAPPPS |
| P322 | C6-HSDAVFTDNYTRLRAibQIAAAibKYLQSIKNKRYGGPSSGAPPPS |
| P323 | C6-HSDAVFTDNYTRLRAibQKAAAibKYLQSIKNKRYGGPSSGAPPPS |
| P324 | C6-HSDAVFTDNYTRLRAibQAAAAibKYLQSIKNKRYGGPSSGAPPPS |
| P325 | C6-HSDAVFTDNYTRLRAibQNleAAAibKYLQSIKNKRYGGPSSGAPPPS |
| P326 | C6-HSDAVFTDNYTOrnLRAibQVAAAibKYLQSIOrnNOrnGGPSSGAPPPS |
| P329 | C6-HSDAVFTDNYThRLRAibQVAAAibKYLQSIhRNhRGGPSSGAPPPS |
| P349 | C6-HSDAVFTDNYTRLRAibQVAAAibKYLQSIKAibKGGPSSGAPPPS |
| P350 | C6-HSDAVFTDNYTRLRAibQVAAAibKYLQSIKPKGGPSSGAPPPS |
| P351 | C6-HSDAVFTDNYTRLRAibQVAAAibKYLQSIKKGGPSSGAPPPS |
| P353 | C6-HSDAVFTDNYTRLRAibQVAAAibKYLQSIdKdKGGPSSGAPPPS |
| P354 | C6-HSDAVFTDNYTRLRAibQVAAAibKYLQSIKhRGGPSSGAPPPS |
| P355 | C6-HSDAVFTDNYTRLRAibQVAAAibKYLQSIKAibGGPSSGAPPPS |
| P364 | C6-HSDAVFTDNYTOrnLRAibQLAAAibKYLQSIOrnNOrnGGPSSGAPPPS |
| P365 | C6-HSDAVFTDNYTOrnLRAibQIAAAibKYLQSIOrnNOrnGGPSSGAPPPS |
| P366 | C6-HSDAVFTDNYTALRAibQVAAAibKYLQSIOrnNOrmGGPSSGAPPPS |

Even more preferred VPAC2 peptide receptor agonists according to the second aspect of the present invention comprise an amino acid sequence selected from:

| | |
|---|---|
| Peptide | Sequence |

| | |
|---|---|
| P167 | C6-HSDAVFTDNYTRLAibKQVAAKKYLQSIKNKRYGGPSSGAPPPS |
| P168 | C6-HSDAVFTDNYTRLRAibQVAAKKYLQSIKNKRYGGPSSGAPPPS |
| P169 | C6-HSDAVFTDNYTRLRKQVAAAibKYLQSIKNKRYGGPSSGAPPPS |
| P170 | C6-HSDAVFTDNYTRLRKQVAAKAibYLQSIKNKRYGGPSSGAPPPS |
| P240 | C6-HSDAVFTDNYTRLAibKQLAAAibKYLQSIKNKRYGGPSSGAPPPS |
| P241 | C6-HSDAVFTDNYTRLAibKQLAAKAibYLQSIKNKRYGGPSSGAPPPS |
| P242 | C6-HSDAVFTDNYTRLAibKQVAAAibKYLQSIKNKRYGGPSSGAPPPS |
| P243 | C6-HSDAVFTDNYTRLAibKQVAAQKYLQSIKNKRYGGPSSGAPPPS |
| P244 | C6-HSDAVFTDNYTRLAibKQVAAKAibYLQSIKNKRYGGPSSGAPPPS |
| P249 | C6-HSDAVFTDNYTRLAibKQVAAKQYLQSIKNKRYGGPSSGAPPPS |
| P250 | C6-HSDAVFTDNYTRLQKQVAAAibKYLQSIKNKRYGGPSSGAPPPS |
| P251 | C6-HSDAVFTDNYTRLQKQVAAKAibYLQSIKNKRYGGPSSGAPPPS |
| P258 | C6-HSDAVFTDNYTRLQAibQVAAKKYLQSIKNKRYGGPSSGAPPPS |
| P259 | C6-HSDAVFTDNYTRLAibKQVAALKYLQSIKNKRYGGPSSGAPPPS |
| P260 | C6-HSDAVFTDNYTRLAibKQVAAAKYLQSIKNKRYGGPSSGAPPPS |
| P261 | C6-HSDAVFTDNYTRLRAibQVAAAibKYLQSIKNKRYGGPSSGAPPPS |
| P262 | C6-HSDAVFTDNYTRLRAibQVAAVKYLQSIKNKRYGGPSSGAPPPS |
| P263 | C6-HSDAVFTDNYTRLRAibQVAAAKYLQSIKNKRYGGPSSGAPPPS |
| P264 | C6-HSDAVFTDNYTRLRAibQVAAKAibYLQSIKNKRYGGPSSGAPPPS |
| P265 | C6-HSDAVFTDNYTRLRAibQVAALKYLQSIKNKRYGGPSSGAPPPS |
| P269 | C6-HSDAVFTDNYTRLAibKQVAAVKYLQSIKNKRYGGPSSGAPPPS |
| P284 | C6-HSDAVFTDNYTRLRAibQLAAKAibYLQSIKNKRYGGPSSGAPPPS |
| P291 | C6-HSDAVFTDNYTRLRAibQLAAAibKYLQSIKNKRYGGPSSGAPPPS |
| P292 | C6-HSDAVFTDNYTRLRAibQLAAAibKYLQSIKNKGGPSSGAPPPS |
| P293 | C6-HSDAVFTDNYTRLAibKQVAAAibKYLQSIKQKGGPSSGAPPPS |
| P294 | C6-HSDAVFTDNYTRLRAibQVAAAibKYLQSIKQKGGPSSGAPPPS |
| P295 | C6-HSDAVFTDNYTRLRAibQVAAKAibYLQSIKQKGGPSSGAPPPS |
| P296 | C6-HSDAVFTDNYTRLAibKQVAAAibKYLQSIKQGGPSSGAPPPS |
| P297 | C6-HSDAVFTDNYTRLRAibQVAAAibKYLQSIKQGGPSSGAPPPS |
| P298 | C6-HSDAVFTDNYTRLRAibQVAAKAibYLQSIKQGGPSSGAPPPS |
| P301 | C6-HSDAVFTDNYTRLAAibQVAAAibKYLQSIKNKRYGGPSSGAPPPS |
| P302 | C6-HSDAVFTDNYTRLQAibQVAAAibKYLQSIKNKRYGGPSSGAPPPS |
| P314 | C6-HSEAVFTENYTRLRAibQVAAAibKYLQSIKNKRYGGPSSGAPPPS |
| P317 | C6-HSDAVFTDNYTRLLAibQVAAAibKYLQSIKNKRYGGPSSGAPPPS |
| P318 | C6-HSDAVFTDNYTRLKAibQVAAAibKYLQSIKNKRYGGPSSGAPPPS |
| P319 | C6-HSDAVFTDNYTRLOrnAibQVAAAibKYLQSIKNKRYGGPSSGAPPPS |
| P321 | C6-HSDAVFTDNYTRLRAibKVAAAibKYLQSIKNKRYGGPSSGAPPPS |
| P322 | C6-HSDAVFTDNYTRLRAibQIAAAibKYLQSIKNKRYGGPSSGAPPPS |
| P323 | C6-HSDAVFTDNYTRLRAibQKAAAibKYLQSIKNKRYGGPSSGAPPPS |
| P324 | C6-HSDAVFTDNYTRLRAibQAAAAibKYLQSIKNKRYGGPSSGAPPPS |
| P325 | C6-HSDAVFTDNYTRLRAibQNleAAAibKYLQSIKNKRYGGPSSGAPPPS |
| P326 | C6-HSDAVFTDNYTOrnLRAibQVAAAibKYLQSIOrnNOrnGGPSSGAPPPS |
| P329 | C6-HSDAVFTDNYThRLRAibQVAAAibKYLQSIhRNhGGPSSGAPPPS |
| P349 | C6-HSDAVFTDNYTRLRAibQVAAAibKYLQSIKAibKGGPSSGAPPPS |
| P350 | C6-HSDAVFTDNYTRLRAibQVAAAibKYLQSIKPKGGPSSGAPPPS |
| P351 | C6-HSDAVFTDNYTRLRAibQVAAAibKYLQSIKKGGPSSGAPPPS |
| P353 | C6-HSDAVFTDNYTRLRAibQVAAAibKYLQSIdKdKGGPSSGAPPPS |
| P354 | C6-HSDAVFTDNYTRLRAibQVAAAibKYLQSIKhRGGPSSGAPPPS |
| P355 | C6-HSDAVFTDNYTRLRAibQVAAAibKYLQSIKAibGGPSSGAPPPS |
| P364 | C6-HSDAVFTDNYTOrnLRAibQLAAAibKYLQSIOrnNOrnGGPSSGAPPPS |
| P365 | C6-HSDAVFTDNYTOrnLRAibQIAAAibKYLQSIOrnNOrnGGPSSGAPPPS |
| P366 | C6-HSDAVFTDNYTALRAibQVAAAibKYLQSIOrnNOrnGGPSSGAPPPS |

According to a third aspect of the present invention, there is provided a VPAC2 receptor peptide agonist comprising a sequence of the formula:

FORMULA 14

(SEQ ID NO: 26)

Xaa₁-Xaa₂-Xaa₃-Xaa₄-Xaa₅-Xaa₆-Thr-Xaa₈-Xaa₉-Xaa₁₀-
Thr-Xaa₁₂-Xaa₁₃-Xaa₁₄-Xaa₁₅-Xaa₁₆-Xaa₁₇-Ala-Xaa₁₉-
Xaa₂₀-Xaa₂₁-Xaa₂₂-Xaa₂₃-Xaa₂₄-Xaa₂₅-Xaa₂₆-Xaa₂₇-
Xaa₂₈-Xaa₂₉-Xaa₃₀-Xaa₃₁-Xaa₃₂-Xaa₃₃-Xaa₃₄-Xaa₃₅-
Xaa₃₆-Xaa₃₇-Xaa₃₈-Xaa₃₉-Xaa₄₀

wherein:
wherein:
Xaa₁ is: any naturally occurring amino acid, dH, or is absent;
Xaa₂ is: any naturally occurring amino acid, dA, dS, or Aib;
Xaa₃ is: Asp or Glu;
Xaa₄ is: any naturally occurring amino acid, dA, Aib, or NMeA;
Xaa₅ is: any naturally occurring amino acid, dV, or Aib;
Xaa₆ is: any naturally occurring amino acid;
Xaa₈ is: Asp, Glu, Ala, Lys, Leu, Arg, or Tyr;
Xaa₉ is: Asn, Gln, Asp, or Glu;
Xaa₁₀ is: any naturally occurring aromatic amino acid, or Tyr (OMe);
Xaa₁₂ is: hR, Orn, Lys (isopropyl), Aib, Cit, or any naturally occurring amino acid except Pro;
Xaa₁₃ is: Aib, or any naturally occurring amino acid except Pro;
Xaa₁₄ is: hR, Orn, Lys (isopropyl), Aib, Cit, or any naturally occurring amino acid except Pro;
Xaa₁₅ is: hR, Orn, Lys (isopropyl), Aib, K(Ac), Cit, or any naturally occurring amino acid except Pro;
Xaa₁₆ is: hR, Orn, Lys (isopropyl), Cit, or any naturally occurring amino acid except Pro;
Xaa₁₇ is: Nle, Aib, or any naturally occurring amino acid except Pro;
Xaa₁₉ is: any naturally occurring amino acid except Pro;
Xaa₂₀ is: hR, Orn, Lys (isopropyl), Aib, K(Ac), Cit, or any naturally occurring amino acid except Pro;
Xaa₂₁ is: hR, Orn, Aib, K(Ac), Cit, or any naturally occurring amino acid except Pro;
Xaa₂₂ is: Aib, Tyr (OMe), or any naturally occurring amino acid except Pro;
Xaa₂₃ is: Aib or any naturally occurring amino acid except Pro;
Xaa₂₄ is: any naturally occurring amino acid except Pro;
Xaa₂₅ is: Aib or any naturally occurring amino acid except Pro;
Xaa₂₆ is: any naturally occurring amino acid except Pro;
Xaa₂₇ is: hR, Lys (isopropyl), Orn, dK, or any naturally occurring amino acid except Pro;
Xaa₂₈ is: any naturally occurring amino acid, Aib, hR, Cit, Orn, or dK;
Xaa₂₉ is: any naturally occurring amino acid, hR, Orn, Cit, Aib, or is absent;
Xaa₃₀ is: any naturally occurring amino acid, hR, Orn, Cit, Aib, or is absent; and
Xaa₃₁ to Xaa₄₀ are any naturally occurring amino acid or are absent;

provided that if Xaa₂₉, Xaa₃₀, Xaa₃₁, Xaa₃₂, Xaa₃₃, Xaa₃₄, Xaa₃₅, Xaa₃₆, Xaa₃₇, Xaa₃₈ or Xaa₃₉ is absent, the next amino acid present downstream is the next amino acid in the peptide agonist and that the peptide agonist comprises at least one amino acid substitution selected from:
Xaa₂ is: dA, Val, Gly, Leu, dS, or Aib;
Xaa₄ is: Ile, Tyr, Phe, Val, Thr, Leu, Trp, dA, Aib, or NMeA;
Xaa₅ is: Leu, Phe, Thr, Trp, Tyr, dV, or Aib;
Xaa₆ is: Ile, Leu, Thr, Val, or Trp;
Xaa₈ is: Leu, Arg, or Tyr;
Xaa₉ is: Glu;
Xaa₁₀ is: Trp;
Xaa₁₂ is: Ala, hR, Aib, Lys (isopropyl), or Cit;
Xaa₁₃ is: Phe, Glu, Ala, or Aib;
Xaa₁₄ is: Leu, Lys, Ala, hR, Orn, Lys (isopropyl), Phe, Gln, Aib, or Cit;
Xaa₁₅ is: Ala, Arg, Leu, hR, Orn, Lys (isopropyl), Phe, Gln, Aib, K(Ac), or Cit;
Xaa₁₆ is: Lys, Lys (isopropyl), hR, Orn, or Cit;
Xaa₁₇ is: Lys, or Aib;
Xaa₂₀ is: Gln, hR, Arg, Ser, Orn, Lys (isopropyl), Ala, Aib, Trp, Thr, Leu, Ile, Phe, Tyr, Val, K(Ac), or Cit;
Xaa₂₁ is: Arg, Ala, Phe, Aib, Leu, Gln, Orn, hR, K(Ac) or Cit;
Xaa₂₂ is: Trp, Thr, Leu, Ile, Val, Tyr (OMe), Ala, or Aib;
Xaa₂₃ is: Phe, Ile, Ala, Trp, Thr, Val, or Aib;
Xaa₂₅ is: Phe, Ile, Leu, Val, Trp, Gln, Asn, Tyr, Aib, or Glu;
Xaa₂₆ is: Thr, Trp, Tyr, or Phe;
Xaa₂₇ is: hR, Orn, or dK;
Xaa₂₈ is: Pro, Arg, Aib, Orn, hR, Cit, or dK;
Xaa₂₉ is: hR, Cys, Orn, Cit, or Aib;
Xaa₃₀ is: hR, Cit, Aib, or Orn; and
Xaa₃₁ is: His, or Phe.

Preferably, the VPAC2 receptor peptide agonist according to the third aspect of the present invention comprises a sequence of the formula:

FORMULA 15

(SEQ ID NO: 27)

His-Xaa₂-Xaa₃-Xaa₄-Xaa₅-Phe-Thr-Xaa₈-Xaa₉-Xaa₁₀-
Thr-Xaa₁₂-Xaa₁₃-Xaa₁₄-Xaa₁₅-Xaa₁₆-Xaa₁₇-Ala-Xaa₁₉-
Xaa₂₀-Xaa₂₁-Xaa₂₂-Xaa₂₃-Xaa₂₄-Xaa₂₅-Xaa₂₆-Xaa₂₇-
Xaa₂₈-Xaa₂₉-Xaa₃₀-Xaa₃₁-Xaa₃₂-Xaa₃₃-Xaa₃₄-Xaa₃₅-
Xaa₃₆-Xaa₃₇-Xaa₃₈-Xaa₃₉-Xaa₄₀

wherein:
Xaa₂ is: dA, Ser, Val, Gly, Thr, Leu, dS, Pro, or Aib;
Xaa₃ is: Asp or Glu;
Xaa₄ is: Ala, Ile, Tyr, Phe, Val, Thr, Leu, Trp, Gly, dA, Aib, or NMeA;
Xaa₅ is: Val, Leu, Phe, Ile, Thr, Trp, Tyr, dV, or Aib;
Xaa₈ is: Asp, Glu, Ala, Lys, Leu, Arg, or Tyr;
Xaa₉ is: Asn, Gln, Asp, or Glu;
Xaa₁₀ is: Tyr, Trp, or Tyr(OMe);
Xaa₁₂ is: Arg, Lys, Glu, hR, Orn, Lys (isopropyl), Aib, Cit, or Ala;
Xaa₁₃ is: Leu, Phe, Glu, Ala, or Aib;
Xaa₁₄ is: Arg, Leu, Lys, Ala, hR, Orn, Lys (isopropyl), Phe, Gln, Aib, or Cit;
Xaa₁₅ is: Lys, Ala, Arg, Glu, Leu, hR, Orn, Lys (isopropyl), Phe, Gln, Aib, K(Ac), or Cit;
Xaa₁₆ is: Gln, Lys, Glu, Ala, hR, Orn, Lys (isopropyl), or Cit;
Xaa₁₇ is: Val, Ala, Leu, Ile, Met, Nle, Lys, or Aib;
Xaa₁₉ is: Val, Ala, Glu, Phe, Gly, His, Ile, Lys, Leu, Met, Asn, Gln, Arg, Ser, Thr, Trp, Tyr, Cys, or Asp;
Xaa₂₀ is: Lys, Gln, hR, Arg, Ser, His, Orn, Lys (isopropyl), Ala, Aib, Trp, Thr, Leu, Ile, Phe, Tyr, Val, K(Ac), or Cit;
Xaa₂₁ is: Lys, His, Arg, Ala, Phe, Aib, Leu, Gln, Orn, hR, K(Ac) or Cit;
Xaa₂₂ is: Tyr, Trp, Phe, Thr, Leu, Ile, Val, Tyr(OMe), Ala, or Aib;
Xaa₂₃ is: Leu, Phe, Ile, Ala, Trp, Thr, Val, or Aib;
Xaa₂₄ is: Gln, Glu, or Asn;
Xaa₂₅ is: Ser, Asp, Phe, Ile, Leu, Thr, Val, Trp, Gln, Asn, Tyr, Aib, or Glu;
Xaa₂₆ is: Ile, Leu, Thr, Val, Trp, Tyr, or Phe;
Xaa₂₇ is: Lys, hR, Arg, Gln, Ala, Asp, Glu, Phe, Gly, His, Ile, Met, Asn, Ser, Thr, Val, Trp, Tyr, Lys (isopropyl), Cys, Leu, Orn, or dK;
Xaa₂₈ is: Asn, Asp, Gln, Lys, Arg, Aib, Orn, hR, Cit, Pro, or dK;
Xaa₂₉ is: Lys, Ser, Arg, Asn, hR, Ala, Asp, Glu, Phe, Gly, His, Ile, Leu, Met, Pro, Gln, Thr, Val, Trp, Tyr, Cys, Orn, Cit, Aib or is absent;
Xaa₃₀ is: Arg, Lys, Ile, Ala, Asp, Glu, Phe, Gly, His, Leu, Met, Asn, Pro, Gln, Ser, Thr, Val, Trp, Tyr, Cys, hR, Cit, Aib, Orn, or is absent;
Xaa₃₁ is: Tyr, His, Phe, Thr, Cys, or is absent;
Xaa₃₂ is: Ser, Cys, or is absent;
Xaa₃₃ is: Trp or is absent;
Xaa₃₄ is: Cys or is absent;
Xaa₃₅ is: Glu or is absent;
Xaa₃₆ is: Pro or is absent;
Xaa₃₇ is: Gly or is absent;
Xaa₃₈ is: Trp or is absent;
Xaa₃₉ is: Cys or is absent; and
Xaa₄₀ is: Arg or is absent

provided that if Xaa₂₉, Xaa₃₀, Xaa₃₁, Xaa₃₂, Xaa₃₃, Xaa₃₄, Xaa₃₅, Xaa₃₆, Xaa₃₇, Xaa₃₈, or Xaa₃₉ is absent, the next amino acid present downstream is the next amino acid in the peptide agonist sequence,

and that the peptide agonist comprises at least one amino acid substitution selected from:
Xaa₂ is: dA, Val, Gly, Leu, dS, or Aib;
Xaa₄ is: Ile, Tyr, Phe, Val, Thr, Leu, Trp, dA, Aib, or NMeA;
Xaa₅ is: Leu, Phe, Thr, Trp, Tyr, dV, or Aib;
Xaa₈ is: Leu, Arg, or Tyr;
Xaa₉ is: Glu;
Xaa₁₀ is: Trp;
Xaa₁₂ is: Ala, hR, Aib, Lys (isopropyl), or Cit;
Xaa₁₃ is: Phe, Glu, Ala, or Aib;
Xaa₁₄ is: Leu, Lys, Ala, hR, Orn, Lys (isopropyl), Phe, Gln, Aib, or Cit;
Xaa₁₅ is: Ala, Arg, Leu, hR, Orn, Lys (isopropyl), Phe, Gln, Aib, K(Ac), or Cit;
Xaa₁₆ is: Lys, Lys (isopropyl), hR, Orn, or Cit;
Xaa₁₇ is: Lys, or Aib;
Xaa₂₀ is: Gln, hR, Arg, Ser, Orn, Lys (isopropyl), Ala, Aib, Trp, Thr, Leu, Ile, Phe, Tyr, Val, K(Ac), or Cit;
Xaa₂₁ is: Arg, Ala, Phe, Aib, Leu, Gln, Orn, hR, K(Ac) or Cit;
Xaa₂₂ is: Trp, Thr, Leu, Ile, Val, Tyr (OMe), Ala, or Aib;
Xaa₂₃ is: Phe, Ile, Ala, Trp, Thr, Val, or Aib;
Xaa₂₅ is: Phe, Ile, Leu, Val, Trp, Gln, Asn, Tyr, Aib, or Glu;
Xaa₂₆ is: Thr, Trp, Tyr, or Phe;
Xaa₂₇ is: hR, Orn, or dK;
Xaa₂₈ is: Pro, Arg, Aib, Orn, hR, Cit, or dK;
Xaa₂₉ is: hR, Cys, Orn, Cit, or Aib;
Xaa₃₀ is: hR, Cit, Aib, or Orn; and
Xaa₃₁ is: His, or Phe.

According to a fourth aspect of the present invention, there is provided a VPAC2 receptor peptide agonist of the present invention for use as a medicament.

According to a further aspect of the present invention, there is provided a VPAC2 receptor peptide agonist of the present invention for use in the manufacture of a medicament for use in the treatment non-insulin-dependent diabetes.

According to yet a further aspect of the present invention, there is provided a VPAC2 receptor peptide agonist of the present invention for use in the manufacture of a medicament for use in the treatment of insulin-dependent diabetes.

Alternative embodiments of the present invention are described below.

A first alternative embodiment of the present invention is a VPAC2 receptor peptide agonist comprising a sequence of the formula:

FORMULA 4

(SEQ ID NO: 7)

Xaa₁-Xaa₂-Xaa₃-Xaa₄-Xaa₅-Xaa₆-Thr-Xaa₈-Xaa₉-Xaa₁₀-
Xaa₁₂-Xaa₁₃-Xaa₁₄-Xaa₁₅-Xaa₁₆-Xaa₁₇-Ala-Xaa₁₉-
Xaa₂₀-Xaa₂₁-Xaa₂₂-Leu-Xaa₂₄-Xaa₂₅-Xaa₂₆-Xaa₂₇-
Xaa₂₈-Xaa₂₉-Xaa₃₀-Xaa₃₁-Xaa₃₂-Xaa₃₃-Xaa₃₄-Xaa₃₅-
Xaa₃₆-Xaa₃₇-Xaa₃₈-Xaa₃₉-Xaa₄₀

wherein:
Xaa₁ is: His or is absent;
Xaa₂ is: dA, Ser, Val, Gly, Thr, Leu, dS, or Pro;
Xaa₃ is: Asp, or Glu;
Xaa₄ is: Ala, Ile, Tyr, Phe, Val, Thr, Leu, Trp, or Gly;
Xaa₅ is: Val, Leu, Phe, Ile, Thr, Trp, or Tyr;
Xaa₆ is: Phe, Ile, Leu, Thr, Val, Trp, or Tyr;
Xaa₈ is: Asp or Glu;
Xaa₉ is: Asn, Gln, or Asp;
Xaa₁₀ is: Tyr or Trp;
Xaa₁₂ is: Arg, Lys, Glu, hR, Orn, or Lys (isopropyl);
Xaa₁₃ is: Leu, Phe, Glu, or Ala;
Xaa₁₄ is: Arg, Leu, Lys, Ala, hR, Orn, or Lys (isopropyl);
Xaa₁₅ is: Lys, Ala, Arg, Glu, Leu, hR, Orn, or Lys (isopropyl);
Xaa₁₆ is: Gln, Lys, Glu, Ala, hR, Orn, or Lys (isopropyl);
Xaa₁₇ is: Val, Ala, Leu, Ile, or Met;
Xaa₁₉ is: Val, Ala, Glu, Phe, Gly, His, Ile, Lys, Leu, Met, Asn, Pro, Gln, Arg, Ser, Thr, Trp, Tyr, Cys, or Asp;
Xaa₂₀ is: Lys, Gln, hR, Arg, Ser, His, Orn, or Lys (isopropyl);
Xaa₂₁ is: Lys, His, or Arg;
Xaa₂₂ is: Tyr, Trp, Phe, Thr, Leu, Ile, or Val;
Xaa₂₄ is: Gln, Glu, or Asn;
Xaa₂₅ is: Ser, Asp, Phe, Ile, Leu, Thr, Val, Trp, Gln, Asn, or Tyr;
Xaa₂₆ is: Ile, Leu, Thr, Val, Trp, Tyr, or Phe;
Xaa₂₇ is: Lys, hR, Arg, Gln, Ala, Asp, Glu, Phe, Gly, His, Ile, Met, Asn, Pro, Ser, Thr, Val, Trp, Tyr, Lys (isopropyl), Cys, or Leu;
Xaa₂₈ is: Asn, Asp, Gln, Lys, or Arg;
Xaa₂₉ is: Lys, Ser, Arg, Asn, hR, Gly, Ala, Asp, Glu, Phe, Gly, His, Ile, Leu, Met, Pro, Gln, Thr, Val, Trp, Tyr, Cys, or is absent;
Xaa₃₀ is: Arg, Lys, Ile, Gly, Ala, Asp, Glu, Phe, Gly, His, Leu, Met, Asn, Pro, Gln, Ser, Thr, Val, Trp, Tyr, Cys, or is absent;
Xaa₃₁ is: Tyr, His, Phe, Thr, Cys, or is absent;
Xaa₃₂ is: Ser, Cys, or is absent;
Xaa₃₃ is: Trp or is absent;
Xaa₃₄ is: Cys or is absent;
Xaa₃₅ is: Glu or is absent;
Xaa₃₆ is: Pro or is absent;
Xaa₃₇ is: Gly or is absent;
Xaa₃₈ is: Trp or is absent;
Xaa₃₉ is: Cys or is absent; and
Xaa₄₀ is: Arg or is absent

provided that if Xaa₂₉, Xaa₃₀, Xaa₃₁, Xaa₃₂, Xaa₃₃, Xaa₃₄, Xaa₃₅, Xaa₃₆, Xaa₃₇, Xaa₃₈, or Xaa₃₉ is absent, the next amino acid present downstream is the next amino acid in the sequence;

and a C-terminal extension wherein the N-terminus of the C-terminal extension is linked to the C-terminus of the sequence and wherein the C-terminal extension comprises an amino acid sequence selected from the group consisting of:

a)

FORMULA 7

(SEQ ID NO: 15)

Xaa₁-Xaa₂-Xaa₃-Xaa₄-Xaa₅-Xaa₆-Xaa₇-Xaa₈-Xaa₉-
Xaa₁₀-Xaa₁₁

wherein:
Xaa₁ is: Gly, Cys, or absent;
Xaa₂ is: Gly, Arg, or absent;
Xaa₃ is: Pro, Thr, or absent;
Xaa₄ is: Ser or absent;
Xaa₅ is: Ser or absent;
Xaa₆ is: Gly or absent;
Xaa₇ is: Ala or absent;
Xaa₈ is: Pro, or absent;
Xaa₉ is: Pro, or absent;
Xaa₁₀ is: Pro or absent; and
Xaa₁₁ is: Ser, Cys, or absent;

provided that if Xaa₁, Xaa₂, Xaa₃, Xaa₄, Xaa₅, Xaa₆, Xaa₇, Xaa₈, Xaa₉, or Xaa₁₀ is absent, the next amino acid present downstream is the next amino acid in the C-terminal extension and wherein the C-terminal amino acid may be amidated;

b)

FORMULA 5

(SEQ ID NO: 8)

Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂-Xaa₁₃

wherein:
Xaa₁ is: Gly or absent;
Xaa₂ is: Gly or absent;
Xaa₃ is: Pro, Ser, Ala, or absent;
Xaa₄ is: Ser, Pro, His, or absent;
Xaa₅ is: Ser, Arg, Thr, Trp, Lys, or absent;
Xaa₆ is: Gly, Ser, or absent;
Xaa₇ is: Ala, Asp, Arg, Glu, Lys, Gly, or absent;
Xaa₈ is: Pro, Ser, Ala, or absent;
Xaa₉ is: Pro, Ser, Ala, or absent;
Xaa₁₀ is: Pro, Ser, Ala, Arg, Lys, His, or absent;
Xaa₁₁ is: Ser, His, Pro, Lys, Arg, or absent;
Xaa₁₂ is: His, Ser, Arg, Lys, or absent; and
Xaa₁₃ is: His, Ser, Arg, Lys, or absent;

provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ is absent, the next amino acid present downstream is the next amino acid in the C-terminal extension and wherein the C-terminal amino acid may be amidated; and

c)

FORMULA 6

(SEQ ID NO: 9)

Xaa₁-Xaa₂-Xaa₃-Xaa₄-Xaa₅-Xaa₆-Xaa₇-Xaa₈-Xaa₉-
Xaa₁₀-Xaa₁₁

wherein:
Xaa₁ is: Gly or absent;
Xaa₂ is: Gly or absent;
Xaa₃ is: Pro, Ser, Ala, or absent;
Xaa₄ is: Ser or absent;
Xaa₅ is: Ser or absent;
Xaa₆ is: Gly or absent;
Xaa₇ is: Ala or absent;
Xaa₈ is: Pro, Ser, Ala;
Xaa₉ is: Pro, Ser, Ala, or absent;
Xaa₁₀ is: Pro, Ser, Ala, or absent; and
Xaa₁₁ is: Ser or absent;

provided that if Xaa₁, Xaa₂, Xaa₃, Xaa₄, Xaa₅, Xaa₆, Xaa₇, Xaa₈, Xaa₉, or Xaa₁₀ is absent, the next amino acid present downstream is the next amino acid in the sequence and wherein the C-terminal amino acid may be amidated.

Preferably, an alternative selective VPAC2 receptor peptide agonist of the present invention has the amino acid sequence of Formula 4 (SEQ ID NO: 7), modified so that from one, two, three, four, five, six, seven, eight, nine, or ten amino acids differ from the amino acid in the corresponding position of SEQ ID NO: 1.

Another alternative embodiment of the present invention is a VPAC2 receptor peptide agonist comprising a sequence of the formula:

FORMULA 2

(SEQ ID NO: 5)

Xaa₁-Xaa₂-Asp-Xaa₄-Xaa₅-Xaa₆-Thr-Xaa₈-Asn-Xaa₁₀-
Thr-Xaa₁₂-Xaa₁₃-Xaa₁₄-Xaa₁₅-Xaa₁₆-Xaa₁₇-Ala-Xaa₁₉-
Xaa₂₀-Xaa₂₁-Xaa₂₂-Leu-Xaa₂₄-Xaa₂₅-Xaa₂₆-Xaa₂₇-
Xaa₂₈-Xaa₂₉-Xaa₃₀-Xaa₃₁

wherein:
Xaa₁ is: His or is absent;
Xaa₂ is: dA, Ser, Val, Gly, Thr, Leu, dS, or Pro;
Xaa₄ is: Ala, Ile, Tyr, Phe, Val, Thr, Leu, Trp, or Gly;
Xaa₅ is: Val, Leu, Phe, Ile, Thr, Trp, or Tyr;
Xaa₆ is: Phe, Ile, Leu, Thr, Val, Trp, or Tyr;
Xaa₈ is: Asp;
Xaa₁₀ is: Tyr or Trp;
Xaa₁₂ is: Arg or Lys;
Xaa₁₃ is: Leu, Phe, Glu, or Ala;
Xaa₁₄ is: Arg, Leu, Lys or Ala;
Xaa₁₅ is: Lys, Ala, Arg, Glu, or Leu;
Xaa₁₆ is: Gln, Lys, or Ala;
Xaa₁₇ is: Val, Ala, Leu, or Met;
Xaa₁₉ is: Ala or Leu;
Xaa₂₀ is: Lys, Gln, hR, Arg, or Ser;
Xaa₂₁ is: Lys or Arg;
Xaa₂₂ is: Tyr, Trp, Phe, Thr, Leu, Ile, or Val;
Xaa₂₄ is: Gln or Asn;
Xaa₂₅ is: Ser, Phe, Ile, Leu, Thr, Val, Trp, Gln, Asn, or Tyr;
Xaa₂₆ is: Ile, Leu, Thr, Val, Trp, Tyr, or Phe;
Xaa₂₇ is: Lys, hR, Arg, Gln, or Leu;
Xaa₂₈ is: Asn, Lys, or Arg;
Xaa₂₉ is: Lys, Ser, Arg, Asn, hR, or is absent;
Xaa₃₀ is: Arg, Lys, Ile, or is absent; and
Xaa₃₁ is: Tyr, His, Phe, or is absent,

provided that if Xaa₂₉ is absent then Xaa₃₀ and Xaa₃₁ are also absent and if Xaa₃₀ is absent then Xaa₃₁ is absent;

and a C-terminal extension wherein the N-terminus of the C-terminal extension is linked to the C-terminus of the sequence and wherein the C-terminal extension comprises an amino acid sequence of the Formula 7 (SEQ ID NO: 15);

provided that if Xaa₁, Xaa₂, Xaa₃, Xaa₄, Xaa₅, Xaa₆, Xaa₇, Xaa₈, Xaa₉, or Xaa₁₀ of Formula 7 is absent, the next amino acid present downstream is the next amino acid in the C-terminal extension and wherein the C-terminal amino acid may be amidated.

Preferably, an alternative selective VPAC2 receptor peptide agonist of the present invention has the amino acid sequence of Formula 2 (SEQ ID NO: 5), modified so that from one, two, three, four, five, six, seven, eight, nine, or ten amino acids differ from the amino acid in the corresponding position of SEQ ID NO: 1.

Yet another alternative embodiment of the present invention is a VPAC2 receptor peptide agonist comprising a sequence of the formula:

FORMULA 3

(SEQ ID NO: 6)

His-Xaa₂-Xaa₃-Ala-Val-Phe-Thr-Xaa₈-Xaa₉-Tyr-Thr-
Xaa₁₂-Leu-Arg-Xaa₁₅-Xaa₁₆-Xaa₁₇-Ala-Xaa₁₉-Xaa₂₀-
Xaa₂₁-Tyr-Leu-Xaa₂₄-Xaa₂₅-Xaa₂₆-Xaa₂₇-Xaa₂₈-Xaa₂₉-
Xaa₃₀-Xaa₃₁-Xaa₃₂-Xaa₃₃-Xaa₃₄-Xaa₃₅-Xaa₃₆-Xaa₃₇-
Xaa₃₈-Xaa₃₉-Xaa₄₀

wherein:
Xaa₂ is: Ser or Thr;
Xaa₃ is: Asp or Glu;
Xaa₈ is: Asp or Glu;
Xaa₉ is: Asn, Gln, or Asp;
Xaa₁₂ is: Arg, Lys, or Glu;
Xaa₁₅ is: Lys or Glu;
Xaa₁₆ is: Gln or Glu;
Xaa₁₇ is: Met, Leu, Ile, or Val;
Xaa₁₉ is: Val, Ala, Glu, Phe, Gly, His, Ile, Lys, Leu, Met, Asn, Pro, Gln, Arg, Ser, Thr, Trp, Tyr, Cys, or Asp;
Xaa₂₀ is: Lys or His;
Xaa₂₁ is: Lys or His;
Xaa₂₄ is: Asn, Gln, or Glu;
Xaa₂₅ is: Ser, Asp, or Thr;
Xaa₂₆ is: Ile or Leu;
Xaa₂₇ is: Leu, Lys, Ala, Asp, Glu, Phe, Gly, His, Ile, Met, Asn, Pro, Gln, Arg, Ser, Thr, Val, Trp, Tyr, or Cys;
Xaa₂₈ is: Asn, Asp, Gln, or Lys;
Xaa₂₉ is: Gly, Lys, Ala, Asp, Glu, Phe, Gly, His, Ile, Leu, Met, Asn, Pro, Gln, Arg, Ser, Thr, Val, Trp, Tyr, Cys, or is absent;
Xaa₃₀ is: Gly, Arg, Ala, Asp, Glu, Phe, Gly, His, Ile, Lys, Leu, Met, Asn, Pro, Gln, Ser, Thr, Val, Trp, Tyr, Cys, or is absent;
Xaa₃₁ is: Thr, Tyr, Cys, or is absent;
Xaa₃₂ is: Ser, Cys, or is absent;
Xaa₃₃ is: Trp or is absent;
Xaa₃₄ is: Cys or is absent;
Xaa₃₅ is: Glu or is absent;
Xaa₃₆ is: Pro or is absent;
Xaa₃₇ is: Gly or is absent;
Xaa₃₈ is: Trp or is absent;
Xaa₃₉ is: Cys or is absent;
Xaa₄₀ is: Arg or is absent;

provided that if Xaa₂₉, Xaa₃₀, Xaa₃₁, Xaa₃₂, Xaa₃₃, Xaa₃₄, Xaa₃₅, Xaa₃₆, Xaa₃₇, Xaa₃₈, or Xaa₃₉ is absent, the next amino acid present downstream is the next amino acid in the sequence;

and a C-terminal extension wherein the N-terminus of the C-terminal extension is linked to the C-terminus of the sequence and wherein the C-terminal extension comprises an amino acid sequence selected from the group consisting of:

a) Formula 7 (SEQ ID NO: 15);

provided that if Xaa₁, Xaa₂, Xaa₃, Xaa₄, Xaa₅, Xaa₆, Xaa₇, Xaa₈, Xaa₉, or Xaa₁₀ of Formula 7 is absent, the next amino acid present downstream is the next amino acid in the C-terminal extension and wherein the C-terminal amino acid may be amidated;

b) Formula 5 (SEQ ID NO: 8);

provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ of Formula 5 is absent, the next amino acid present downstream is the next amino acid in the C-terminal extension and wherein the C-terminal amino acid may be amidated; and

c) Formula 6 (SEQ ID NO: 9);

provided that if Xaa₁, Xaa₂, Xaa₃, Xaa₄, Xaa₅, Xaa₆, Xaa₇, Xaa₈, Xaa₉, or Xaa₁₀ of Formula 6 is absent, the next amino acid present downstream is the next amino acid in the sequence and wherein the C-terminal amino acid may be amidated.

For example, if Xaa₂₉ of the peptide sequence is Gly and Xaa₃₀ is absent, the next amino acid bonded to Gly at position 29 is an amino acid listed for position 31 or, if position 31 is also absent, an amino acid listed for position 32 is bonded to Gly at position 29, and so forth. Additionally, for example, if Xaa₂₉ is Gly and Xaa₃₀ through Xaa₄₀ are absent, Gly may be the C-terminal amino acid and may be amidated.

Preferably, an alternative selective VPAC2 receptor peptide agonist of the present invention has the amino acid sequence of Formula 3 (SEQ ID NO: 6), modified so that from one, two, three, four, five, six, seven, eight, nine, or ten amino acids differ from the amino acid in the corresponding position of SEQ ID NO: 1.

Another alternative embodiment of the present invention is a VPAC2 receptor peptide agonist comprising a sequence of the formula:

Formula 1

(SEQ ID NO: 4)

His-Xaa₂-Asp-Ala-Val-Phe-Thr-Asp-Asn-Tyr-Thr-
Xaa₁₂-Leu-Xaa₁₄-Xaa₁₅-Xaa₁₆-Xaa₁₇-Ala-Xaa₁₉-Xaa₂₀-
Xaa₂₁-Tyr-Leu-Xaa₂₄-Xaa₂₅-Xaa₂₆-Xaa₂₇-Asn-Xaa₂₉-
Xaa₃₀-Xaa₃₁

wherein:
Xaa₂ is: Ser, Val, dA, or dS;
Xaa₁₂ is: Arg, Lys, hR, Orn, or Lys (isopropyl);
Xaa₁₄ is: Arg, Leu, Lys, hR, Orn, or Lys (isopropyl);
Xaa₁₅ is: Lys, Ala, Arg, hR, Orn, or Lys (isopropyl);
Xaa₁₆ is: Gln, Lys, Ala, hR, Orn, or Lys (isopropyl);
Xaa₁₇ is: Met, Val, Ala, or Leu;
Xaa₁₉ is: Val, Ala or Leu;
Xaa₂₀ is: Lys, Gln, Arg, hR, Orn, or Lys (isopropyl);
Xaa₂₁ is: Lys or Arg;
Xaa₂₄ is: Asn or Gln;
Xaa₂₅ is: Ser, Phe, Ile, Leu, Thr, Val, Trp, Gln, Asn, or Tyr;
Xaa₂₆ is: Ile, Leu, Thr, Val, Trp, Tyr, or Phe;
Xaa₂₇ is: Leu, hR, Arg, Lys, or Lys (isopropyl);
Xaa₂₉ is: Lys, Ser, Arg, hR, or absent;
Xaa₃₀ is: Arg, Lys, or absent; and
Xaa₃₁ is: Tyr, Phe, or absent,

provided that at least one Xaa selected from the group consisting of: Xaa₂, Xaa₁₄, Xaa₁₅, Xaa₁₆, Xaa₁₇, Xaa₂₀, Xaa₂₅, Xaa₂₆, Xaa₂₇, and Xaa₃₁ is an amino acid that differs from the amino acid at the corresponding position in SEQ ID NO: 1,

provided that if Xaa₂₉ is absent then Xaa₃₀ and Xaa₃₁ are also absent, and if Xaa₃₀ is absent then Xaa₃₁ is also absent;

and a C-terminal extension wherein the N-terminus of the C-terminal extension is linked to the C-terminus of the sequence and wherein the C-terminal extension comprises an amino acid sequence selected from the group consisting of:

a) Formula 5 (SEQ ID NO: 8);

provided that if Xaa₁, Xaa₂, Xaa₃, Xaa₄, Xaa₅, Xaa₆, Xaa₇, Xaa₈, Xaa₉, Xaa₁₀, Xaa₁₁, Xaa₁₂, or Xaa₁₃ of Formula 5 is absent, the next amino acid present downstream is the next amino acid in the C-terminal extension and wherein the C-terminal amino acid may be amidated; and

b) Formula 6 (SEQ ID NO: 9);

provided that if Xaa₁, Xaa₂, Xaa₃, Xaa₄, Xaa₅, Xaa₆, Xaa₇, Xaa₈, Xaa₉, or Xaa₁₀ of Formula 6 is absent, the next amino acid present downstream is the next amino acid in the sequence and wherein the C-terminal amino acid may be amidated.

Preferably, an alternative selective VPAC2 receptor peptide agonist of the present invention has the amino acid sequence of Formula 1 (SEQ ID NO: 4), modified so that from one, two, three, four, five, six, seven, eight, nine, or ten amino acids differ from the amino acid in the corresponding position of SEQ ID NO: 1.

A further alternative embodiment of the present invention is a VPAC2 receptor peptide agonist comprising a sequence of the Formula 1 (SEQ ID NO: 4), wherein the sequence has at least one amino acid substitution selected from the group consisting of:
Xaa₂ is: Val or dA;
Xaa₁₄ is: Leu;
Xaa₁₅ is: Ala;
Xaa₁₆ is: Lys;
Xaa₁₇ is: Ala;
Xaa₂₀ is: Gln;
Xaa₂₅ is: Phe, Ile, Leu, Val, Trp, or Tyr;
Xaa₂₆ is: Thr, Trp, or Tyr;
Xaa₂₇ is: hR; and
Xaa₃₁ is: Phe,

and provided that if Xaa₂₉ is absent then Xaa₃₀ and Xaa₃₁ are also absent and if Xaa₃₀ is absent then Xaa₃₁ is absent.

The peptide of Formula 1 (SEQ ID NO: 4) can further comprise a C-terminal extension wherein the N-terminus of the C-terminal extension is linked to the C-terminus of the peptide of Formula 1 (SEQ ID NO: 4) and wherein the C-terminal extension comprises an amino acid sequence selected from the group consisting of:

a) Formula 5 (SEQ ID NO: 8);

provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ of Formula 5 is absent, the next amino acid present downstream is the next amino acid in the C-terminal extension and wherein the C-terminal amino acid may be amidated; and

b) Formula 6 (SEQ ID NO: 9);

provided that if Xaa₁, Xaa₂, Xaa₃, Xaa₄, Xaa₅, Xaa₆, Xaa₇, Xaa₈, Xaa₉, or Xaa₁₀ of Formula 6 is absent, the next amino acid present downstream is the next amino acid in the sequence and wherein the C-terminal amino acid may be amidated.

Another alternative embodiment of the present invention is a VPAC2 receptor peptide agonist comprising a sequence of the formula:

Formula 8

(SEQ ID NO: 16)

Xaa₁-Xaa₂-Xaa₃-Xaa₄-Xaa₅-Xaa₆-Thr-Xaa₈-Xaa₉-Xaa₁₀-
Thr-Xaa₁₂-Xaa₁₃-Xaa₁₄-Xaa₁₅-Xaa₁₆-Xaa₁₇-Ala-Xaa₁₉-
Xaa₂₀-Xaa₂₁-Xaa₂₂-Leu-Xaa₂₄-Xaa₂₅-Xaa₂₆-Xaa₂₇-
Xaa₂₈-Xaa₂₉-Xaa₃₀-Xaa₃₁-Xaa₃₂-Xaa₃₃-Xaa₃₄-Xaa₃₅-
Xaa₃₆-Xaa₃₇-Xaa₃₈-Xaa₃₉-Xaa₄₀

wherein:
Xaa₁ is: His or is absent;
Xaa₂ is: dA, Ser, Val, Gly, Thr, Leu, dS, Pro, or Aib;
Xaa₃ is: Asp or Glu;
Xaa₄ is: Ala, Ile, Tyr, Phe, Val, Thr, Leu, Trp, Gly, dA, or Aib;
Xaa₅ is: Val, Leu, Phe, Ile, Thr, Trp, Tyr, dV, or Aib;
Xaa₆ is: Phe, Ile, Leu, Thr, Val, Trp, or Tyr;
Xaa₈ is: Asp, Glu, or Ala;
Xaa₉ is: Asn, Gln, Asp, or Glu;
Xaa₁₀ is: Tyr, Trp, or Tyr(OMe);
Xaa₁₂ is: Arg, Lys, Glu, hR, Orn, or Lys (isopropyl);
Xaa₁₃ is: Leu, Phe, Glu, or Ala;
Xaa₁₄ is: Arg, Leu, Lys, Ala, hR, Orn, Lys (isopropyl), Phe, Gln, or Aib;
Xaa₁₅ is: Lys, Ala, Arg, Glu, Leu, hR, Orn, Lys (isopropyl), Phe, Gln, or Aib;
Xaa₁₆ is: Gln, Lys, Glu, Ala, hR, Orn, or Lys (isopropyl);
Xaa₁₇ is: Val, Ala, Leu, Ile, Met, or Nle;
Xaa₁₉ is: Val, Ala, Glu, Phe, Gly, His, Ile, Lys, Leu, Met, Asn, Pro, Gln, Arg, Ser, Thr, Trp, Tyr, Cys, or Asp;
Xaa₂₀ is: Lys, Gln, hR, Arg, Ser, His, Orn, or Lys (isopropyl), Ala, Aib, Trp, Thr, Leu, Ile, Phe, Tyr, or Val;
Xaa₂₁ is: Lys, His, Arg, Ala, Phe, Aib, Leu, or Gln;
Xaa₂₂ is: Tyr, Trp, Phe, Thr, Leu, Ile, or Val;
Xaa₂₄ is: Gln, Glu, or Asn;
Xaa₂₅ is: Ser, Asp, Phe, Ile, Leu, Thr, Val, Trp, Gln, Asn, Tyr, or Aib;
Xaa₂₆ is: Ile, Leu, Thr, Val, Trp, Tyr, Phe or Aib;
Xaa₂₇ is: Lys, hR, Arg, Gln, Ala, Asp, Glu, Phe, Gly, His, Ile, Met, Asn, Pro, Ser, Thr, Val, Trp, Tyr, Lys (isopropyl), Cys, or Leu;
Xaa₂₈ is: Asn, Asp, Gln, Lys, or Arg;
Xaa₂₉ is: Lys, Ser, Arg, Asn, hR, Gly, Ala, Asp, Glu, Phe, Gly, His, Ile, Leu, Met, Pro, Gln, Thr, Val, Trp, Tyr, Cys, or is absent;
Xaa₃₀ is: Arg, Lys, Ile, Gly, Ala, Asp, Glu, Phe, Gly, His, Leu, Met, Asn, Pro, Gln, Ser, Thr, Val, Trp, Tyr, Cys, or is absent;
Xaa₃₁ is: Tyr, His, Phe, Thr, Cys, or is absent;
Xaa₃₂ is: Ser, Cys, or is absent;
Xaa₃₃ is: Trp or is absent;
Xaa₃₄ is: Cys or is absent;
Xaa₃₅ is: Glu or is absent;
Xaa₃₆ is: Pro or is absent;
Xaa₃₇ is: Gly or is absent;
Xaa₃₈ is: Trp or is absent;
Xaa₃₉ is: Cys or is absent; and
Xaa₄₀ is: Arg or is absent

provided that if Xaa₂₉, Xaa₃₀, Xaa₃₁, Xaa₃₂, Xaa₃₃, Xaa₃₄, Xaa₃₅, Xaa₃₆, Xaa₃₇, Xaa₃₈, or Xaa₃₉ is absent, the next amino acid present downstream is the next amino acid in the sequence;

and a C-terminal extension wherein the N-terminus of the C-terminal extension is linked to the C-terminus of the sequence and wherein the C-terminal extension comprises an amino acid sequence selected from the group consisting of:

a) Formula 7 (SEQ ID NO: 15);

provided that if Xaa₁, Xaa₂, Xaa₃, Xaa₄, Xaa₅, Xaa₆, Xaa₇, Xaa₈, Xaa₉, or Xaa₁₀ of Formula 7 is absent, the next amino acid present downstream is the next amino acid in the C-terminal extension and wherein the C-terminal amino acid may be amidated;

b) Formula 5 (SEQ ID NO: 8);

provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ of Formula 5 is absent, the next amino acid present downstream is the next amino acid in the C-terminal extension and wherein the C-terminal amino acid may be amidated; and

c) Formula 6 (SEQ ID NO: 9);

provided that if Xaa₁, Xaa₂, Xaa₃, Xaa₄, Xaa₅, Xaa₆, Xaa₇, Xaa₈, Xaa₉, or Xaa₁₀ of Formula 6 is absent, the next amino acid present downstream is the next amino acid in the sequence and wherein the C-terminal amino acid may be amidated.

Preferably, an alternative selective VPAC2 receptor peptide agonist of the present invention has the amino acid sequence of Formula 8 (SEQ ID NO: 16), modified so that from one, two, three, four, five, six, seven, eight, nine, or ten amino acids differ from the amino acid in the corresponding position of SEQ ID NO: 1.

Another alternative embodiment of the present invention is a VPAC2 receptor peptide agonist comprising a sequence of the formula:

Formula 9

(SEQ ID NO: 17)

Xaa₁-Xaa₂-Xaa₃-Xaa₄-Xaa₅-Xaa₆-Thr-Xaa₈-Xaa₉-Xaa₁₀-
Thr-Xaa₁₂-Xaa₁₃-Xaa₁₄-Xaa₁₅-Xaa₁₆-Xaa₁₇-Ala-Xaa₁₉-
Xaa₂₀-Xaa₂₁-Xaa₂₂-Xaa₂₃-Xaa₂₄-Xaa₂₅-Xaa₂₆-Xaa₂₇-
Xaa₂₈-Xaa₂₉-Xaa₃₀-Xaa₃₁-Xaa₃₂-Xaa₃₃-Xaa₃₄-Xaa₃₅-
Xaa₃₆-Xaa₃₇-Xaa₃₈-Xaa₃₉-Xaa₄₀

wherein:
Xaa₁ is: His or is absent;
Xaa₂ is: dA, Ser, Val, Gly, Thr, Leu, dS, Pro, or Aib;
Xaa₃ is: Asp or Glu;
Xaa₄ is: Ala, Ile, Tyr, Phe, Val, Thr, Leu, Trp, Gly, dA, Aib, or NMeA;
Xaa₅ is: Val, Leu, Phe, Ile, Thr, Trp, Tyr, dV, Aib, or NMeV;
Xaa₆ is: Phe, Ile, Leu, Thr, Val, Trp, or Tyr;
Xaa₈ is: Asp, Glu, Ala, Lys, Leu, Arg, or Tyr;
Xaa₉ is: Asn, Gln, Asp, or Glu;
Xaa₁₀ is: Tyr, Trp, or Tyr(OMe);
Xaa₁₂ is: Arg, Lys, Glu, hR, Orn, or Lys (isopropyl);
Xaa₁₃ is: Leu, Phe, Glu, or Ala;
Xaa₁₄ is: Arg, Leu, Lys, Ala, hR, Orn, Lys (isopropyl), Phe, Gln, or Aib;
Xaa₁₅ is: Lys, Ala, Arg, Glu, Leu, hR, Orn, Lys (isopropyl), Phe, Gln, or Aib;
Xaa₁₆ is: Gln, Lys, Glu, Ala, hR, Orn, or Lys (isopropyl);
Xaa₁₇ is: Val, Ala, Leu, Ile, Met, or Nle;
Xaa₁₉ is: Val, Ala, Glu, Phe, Gly, His, Ile, Lys, Leu, Met, Asn, Pro, Gln, Arg, Ser, Thr, Trp, Tyr, Cys, or Asp;
Xaa₂₀ is: Lys, Gln, hR, Arg, Ser, His, Orn, or Lys (isopropyl), Ala, Aib, Trp, Thr, Leu, Ile, Phe, Tyr, or Val;
Xaa₂₁ is: Lys, His, Arg, Ala, Phe, Aib, Leu, or Gln;
Xaa₂₂ is: Tyr, Trp, Phe, Thr, Leu, Ile, Val, Tyr(OMe), Ala, or Aib;
Xaa₂₃ is: Leu, Phe, Ile, Ala, Trp, Thr, Val, or Aib;
Xaa₂₄ is: Gln, Glu, or Asn;
Xaa₂₅ is: Ser, Asp, Phe, Ile, Leu, Thr, Val, Trp, Gln, Asn, Tyr, or Aib;
Xaa₂₆ is: Ile, Leu, Thr, Val, Trp, Tyr, Phe or Aib;
Xaa₂₇ is: Lys, hR, Arg, Gln, Ala, Asp, Glu, Phe, Gly, His, Ile, Met, Asn, Pro, Ser, Thr, Val, Trp, Tyr, Lys (isopropyl), Cys, or Leu;
Xaa₂₈ is: Asn, Asp, Gln, Lys, or Arg;
Xaa₂₉ is: Lys, Ser, Arg, Asn, hR, Gly, Ala, Asp, Glu, Phe, Gly, His, Ile, Leu, Met, Pro, Gln, Thr, Val, Trp, Tyr, Cys, or is absent;
Xaa₃₀ is: Arg, Lys, Ile, Gly, Ala, Asp, Glu, Phe, Gly, His, Leu, Met, Asn, Pro, Gln, Ser, Thr, Val, Trp, Tyr, Cys, or is absent;
Xaa₃₁ is: Tyr, His, Phe, Thr, Cys, or is absent;
Xaa₃₂ is: Ser, Cys, or is absent;
Xaa₃₃ is: Trp or is absent;
Xaa₃₄ is: Cys or is absent;
Xaa₃₅ is: Glu or is absent;
Xaa₃₆ is: Pro or is absent;
Xaa₃₇ is: Gly or is absent;
Xaa₃₈ is: Trp or is absent;
Xaa₃₉ is: Cys or is absent; and
Xaa₄₀ is: Arg or is absent

provided that if Xaa₂₉, Xaa₃₀, Xaa₃₁, Xaa₃₂, Xaa₃₃, Xaa₃₄, Xaa₃₅, Xaa₃₆, Xaa₃₇, Xaa₃₈, or Xaa₃₉ is absent, the next amino acid present downstream is the next amino acid in the sequence;

and a C-terminal extension wherein the N-terminus of the C-terminal extension is linked to the C-terminus of the sequence and wherein the C-terminal extension comprises an amino acid sequence selected from the group consisting of:

a) Formula 7 (SEQ ID NO: 15);

provided that if Xaa₁, Xaa₂, Xaa₃, Xaa₄, Xaa₅, Xaa₆, Xaa₇, Xaa₈, Xaa₉, or Xaa₁₀ of Formula 7 is absent, the next amino acid present downstream is the next amino acid in the C-terminal extension and wherein the C-terminal amino acid may be amidated;

b) Formula 5 (SEQ ID NO: 8);

provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ of Formula 5 is absent, the next amino acid present downstream is the next amino acid in the C-terminal extension and wherein the C-terminal amino acid may be amidated; and

c) Formula 6 (SEQ ID NO: 9);

provided that if Xaa₁, Xaa₂, Xaa₃, Xaa₄, Xaa₅, Xaa₆, Xaa₇, Xaa₈, Xaa₉, or Xaa₁₀ of Formula 6 is absent, the next amino acid present downstream is the next amino acid in the sequence and wherein the C-terminal amino acid may be amidated.

Preferably, an alternative selective VPAC2 receptor peptide agonist of the present invention has the amino acid sequence of Formula 9 (SEQ ID NO: 17), modified so that from one, two, three, four, five, six, seven, eight, nine, or ten amino acids differ from the amino acid in the corresponding position of SEQ ID NO: 1.

Additional alternative embodiments of the present invention include a VPAC2 receptor peptide agonist further comprising a N-terminal modification linked to the N-terminus of the peptide sequence wherein the N-terminal modification involves acylation, alkylation, acetylation, a carbobenzoyl group, a succinimide group, a sulfonamide group, a carbamate group, or a urea group. N-terminal modification includes, but is not limited to eighteen carbons (C-18), ten carbons (C-10), and six carbons (C-6). N-terminal modification also includes HS(CH₂)₂CO.

Other alternative embodiments of the present invention include a VPAC2 receptor peptide agonist further comprising a N-terminal modification linked to the N-terminus of the peptide sequence wherein the N-terminal modification is selected from the group consisting of D-histidine and isoleucine.

Alternative embodiments of the present invention also include a VPAC2 receptor peptide agonist further comprising a N-terminal modification linked to the N-terminus of the peptide sequence wherein the N-terminal modification is selected from the group consisting of acetyl, propionyl, butyryl, pentanoyl, hexanoyl, Met, 3-phenylpropionyl, phenylacetyl, benzoyl, and norleucine.

The VPAC2 receptor peptide agonists of the present invention, therefore, have the advantage that they have enhanced selectivity, potency and/or stability over known VPAC2 receptor peptide agonists. In particular, the addition of the extension sequence of exendin-4 as the c-capping sequence surprisingly increased the VPAC2 receptor selectivity as well as increasing proteolytic stability.

A “selective VPAC2 receptor peptide agonist” of the present invention is a peptide that selectively activates the VPAC2 receptor to induce insulin secretion. Preferably, the sequence for a selective VPAC2 receptor peptide agonist of the present invention has from about twenty-five to about thirty-five naturally occurring and/or non-naturally occurring amino acids. More preferably, this sequence has from twenty-eight to thirty-one naturally occurring and/or non-naturally occurring amino acids.

Optionally, the selective VPAC2 receptor peptide agonist can also have an N-terminal modification. Examples include adding one or more naturally occurring or non-naturally occurring amino acids or acylation of the N-terminus.

The N-terminal modification for the peptides of the present invention may comprise the addition of one or more naturally occurring or non-naturally occurring amino acids to the VPAC2 receptor peptide agonist sequence, preferably not more than ten amino acids, with one amino acid being more preferred. Naturally occurring amino acids which may be added to the N-terminus include methionine and isoleucine. A modified amino acid added to the N-terminus may be D-histidine. Alternatively, the following amino acids may be added to the N-terminus: SEQ ID NO: 618 Ser-Trp-Cys-Glu-Pro-Gly-Trp-Cys-Arg, wherein the Arg is linked to the N-terminus of the peptide agonist. Preferably, any amino acids added to the N-terminus are linked to the N-terminus by a peptide bond.

The term “linked to” as used herein, with reference to the term N-terminal modification, includes the addition or attachment of amino acids or chemical groups directly to the N-terminus of the VPAC2 receptor agonist. The addition of the above N-terminal modifications is usually achieved under normal coupling conditions for peptide bond formation.

The N-terminus of the peptide agonist may also be modified by the addition of an alkyl group (R), preferably a C₁-C₁₆ alkyl group, to form (R)NH—.

Alternatively, the N-terminus of the peptide agonist may be modified by the addition of a group of the formula —C(O)R¹ to form an amide of the formula R¹C(O)NH—. The addition of a group of the formula —C(O)R¹ may be achieved by reaction with an organic acid of the formula R¹COOH. Modification of the N-terminus of an amino acid sequence using acylation is demonstrated in the art (e.g. Gozes et al., J. Pharmacol Exp Ther, 273:161-167 (1995)). Addition of a group of the formula —C(O)R¹ may result in the formation of a urea group (see WO 01/23240, WO 2004/006839) or a carbamate group at the N-terminus.

The N-terminus of the peptide agonist may be modified by the addition of a group of the formula —SO₂R⁵, to form a sulfonamide group at the N-terminus.

The N-terminus of the peptide agonist may also be modified by reacting with succinic anhydride to form a succinimide group at the N-terminus. The succinimide group incorporates the nitrogen at the N-terminal of the peptide.

The N-terminus may alternatively be modified by the addition of methionine sulfoxide.

Selective VPAC2 receptor peptide agonists may also have an optional C-terminal extension. The “C-terminal extension” of the present invention comprises a sequence having from one to thirteen naturally occurring or non-naturally occurring amino acids linked to the C-terminus of the sequence at the N-terminus of the C-terminal extension via a peptide bond.

As used herein, the term “linked to” with reference to the term C-terminal extension, includes the addition or attachment of amino acids or chemical groups directly to the C-terminus of the peptide of the Formula 10, 12 or 13.

Most of the sequences of the present invention, including the N-terminal modifications and the C-terminal extensions contain the standard single letter codes for the twenty naturally occurring amino acids. The other codes used are defined as follows:

Ac=Acetyl

C6=hexanoyl

d=the D isoform (nonnaturally occurring) of the respective amino acid,
e.g., dA=D-alanine, dS=D-serine, dK=D-lysine

hR=homoarginine

_=position not occupied

Aib=amino isobutyric acid

CH2=ethylene

Met(O)=methionine sulfoxide

OMe=methoxy

Nle=Nor-leucine

NMe=N-methyl attached to the alpha amino group of an amino acid,
e.g., NMeA=N-methyl alanine, NMeV=N-methyl valine

Orn=ornithine

Cit=citrulline

K(Ac)=ε-acetyl lysine.

M=methionine

I=isoleucine

The term “VPAC2” is used to refer to and in conjunction with the particular receptor (see Lutz, 1999; Adamou, 1995) that the agonists of the present invention activate. This term also is used to refer to and in conjunction with the agonists of the present invention.

VIP naturally occurs as a single sequence having 28 amino acids. However, PACAP exists as either a 38 amino acid peptide (PACAP-38) or as a 27 amino acid peptide (PACAP-27) with an amidated carboxyl (Miyata, et al., Biochem Biophys Res Commun, 170:643-648 (1990)). The sequences for VIP, PACAP-27, and PACAP-38 are as follows:

| | | |
|---|---|---|
| Peptide | Seq. ID # | Sequence |

| | | |
|---|---|---|
| VIP | SEQ ID NO: 1 | HSDAVFTDNYTRLRKQMAVKKYLNSILN |
| PACAP-27 | SEQ ID NO: 2 | HSDGIFTDSYSRYRKQMAVKKYLAAVL- |
| | | NH₂ |
| PACAP-38 | SEQ ID NO: 3 | HSDGIFTDSYSRYRKQMAVKKYLAAVLGK |
| | | RYQRVKNK-NH₂ |

The term “naturally occurring amino acid” as used herein means the twenty amino acids coded for by the human genetic code (i.e. the twenty standard amino acids). These twenty amino acids are: Alanine, Arginine, Asparagine, Aspartic Acid, Cysteine, Glutamine, Glutamic Acid, Glycine, Histidine, Isoleucine, Leucine, Lysine, Methionine, Phenylalanine, Proline, Serine, Threonine, Tryptophan, Tyrosine and Valine.

Examples of “non-naturally occurring amino acids” include both synthetic amino acids and those modified by the body. These include D-amino acids, arginine-like amino acids (e.g., homoarginine), and other amino acids having an extra methylene in the side chain (“homo” amino acids), and modified amino acids (e.g norleucine, lysine (isopropyl)—wherein the side chain amine of lysine is modified by an isopropyl group). Also included are amino acids such as ornithine and amino isobutyric acid. Preferably, however, the selective VPAC2 receptor peptide agonists of the present invention most frequently comprise naturally occurring amino acids except as otherwise specifically provided herein.

“Selective” as used herein refers to a VPAC2 receptor peptide agonist with increased selectivity for the VPAC2 receptor compared to other known receptors. The degree of selectivity is determined by a ratio of VPAC2 receptor binding affinity to VPAC1 receptor binding affinity and by a ratio of VPAC2 receptor binding affinity to PAC1 receptor binding affinity. Preferably, the agonists of the present invention have a selectivity ratio where the affinity for the VPAC2 receptor is at least 50 times greater than for the VPAC1 and/or for PAC1 receptors. More preferably, the affinity is at least 100 times greater for VPAC2 than for VPAC1 and/or for PAC1. Even more preferably, the affinity is at least 200 times greater for VPAC2 than for VPAC1 and/or for PAC1. Still more preferably, the affinity is at least 500 times greater for VPAC2 than for VPAC1 and/or for PAC1. Yet more preferably, the affinity is at least 1000 times greater for VPAC2 than for VPAC1 and/or for PAC1. Binding affinity is determined as described below in Example 4.

“Percent (%) sequence identity” as used herein is used to denote sequences which when aligned have similar (identical or conservatively replaced) amino acids in like positions or regions, where identical or conservatively replaced amino acids are those which do not alter the activity or function of the protein as compared to the starting protein. For example, two amino acid sequences with at least 85% identity to each other have at least 85% similar (identical or conservatively replaced residues) in a like position when aligned optimally allowing for up to 3 gaps, with the proviso that in respect of the gaps a total of not more than 15 amino acid residues is affected. Percent sequence identity may be calculated by determining the number of residues that differ between a peptide encompassed by the present invention and a reference peptide such as VIP, taking that number and dividing it by the number of amino acids in the reference peptide (e.g. 28 amino acids for VIP), multiplying the result by 100, and subtracting that resulting number from 100. For example, a sequence having 28 amino acids with four amino acids that are different from VIP would have a percent (%) sequence identity of 86% (e.g. 100−((4/28)×100)). For a sequence that is longer than 28 amino acids, the number of residues that differ from the VIP sequence will include the additional amino acids over 28 for purposes of the aforementioned calculation. For example, a sequence having 31 amino acids, with four amino acids different from the 28 amino acids in the VIP sequence and with three additional amino acids at the carboxy terminus which are not present in the VIP sequence, would have a total of seven amino acids that differ from VIP. Thus, this sequence would have a percent (%) sequence identity of 75% (e.g. 100−((7/28)×100)). The degree of sequence identity may be determined using methods well known in the art (see, for example, Wilbur, W. J. and Lipman, D. J. “Rapid Similarity Searches of Nucleic Acid and Protein Data Banks”, “Proceedings of the National Academy of Sciences USA 80, 726-730 (1983)” and Myers E. and Miller W. “Optimal Alignments in Linear Space” Comput. Appl. Biosci. 4:11-17 (1988)). One program which may be used in determining the degree of similarity is the MegAlign Lipman-Pearson one pair method (using default parameters) which can be obtained from DNAstar Inc, 1128, Selfpark Street, Madison, Wis., 53715, USA as part of the Lasergene system. Another program, which may be used, is Clustal W. This is a multiple sequence alignment package developed by Thompson et al (Nucleic Acids Research, 1994, Vol. 22, No. 22, 4673-4680) for DNA or protein sequences. This tool is useful for performing cross-species comparisons of related sequences and viewing sequence conservation. Clustal W is a general purpose multiple sequence alignment program for DNA or proteins. It produces biologically meaningful multiple sequence alignments of divergent sequences. It calculates the best match for the selected sequences, and lines them up so that the identities, similarities and differences can be seen. Evolutionary relationships can be seen via viewing Cladograms or Phylograms.

The sequence for selective VPAC2 receptor peptide agonists of the present invention are selective for the VPAC2 receptor and preferably has a sequence identity in the range of 60% to 70%, 60% to 65%, 65% to 70%, 70% to 80%, 70% to 75%, 75% to 80%, 80% to 90%, 80% to 85%, 85% to 90%, 90% to 97%, 90% to 95%, or 95% to 97%, with VIP (SEQ ID NO: 1). More preferably, the sequence has about 61%, 64%, 68%, 71%, 75%, 79%, 82%, 86%, 89%, 93%, or 96% sequence identity with VIP.

The term “C₁-C₁₆ alkyl” as used herein means a monovalent saturated straight, branched or cyclic chain hydrocarbon radical having from 1 to 16 carbon atoms. Thus the term “C₁-C₁₆ alkyl” includes, for example, methyl, ethyl, n-propyl, isopropyl, n-butyl, isobutyl, sec-butyl, tert-butyl, n-heptyl, n-octyl, cyclopropyl, cyclobutyl, cyclopentyl and cyclohexyl. The C₁-C₁₆ alkyl group may be optionally substituted with one or more substituents.

The term “C₁-C₆ alkyl” as used herein means a monovalent saturated straight-chain, branched or cyclic chain hydrocarbon radical having from 1 to 6 carbon atoms. Thus the term “C₁-C₆ alkyl” includes, for example, methyl, ethyl, n-propyl, isopropyl, n-butyl, isobutyl, sec-butyl, tert-butyl, cyclopropyl, cyclobutyl, cyclopentyl and cyclohexyl. The C₁-C₆ alkyl group may be optionally substituted with one or more substituents.

The term “C₂-C₆ alkenyl” as used herein means a monovalent straight, branched or cyclic chain hydrocarbon radical having at least one double bond and having from 2 to 6 carbon atoms. Thus the term “C₂-C₆ alkenyl” includes vinyl, prop-2-enyl, but-3-enyl, pent-4-enyl and isopropenyl. The C₂-C₆ alkenyl group may be optionally substituted with one or more substituents.

The term “C₂-C₆ alkynyl” as used herein means a monovalent straight or branched chain hydrocarbon radical having at least one triple bond and having from 2 to 6 carbon atoms. Thus the term “C₂-C₆ alkynyl” includes prop-2-ynyl, but-3-ynyl and pent-4-ynyl. The C₂-C₆ alkynyl may be optionally substituted with one or more substituents.

The term “halo” or “halogen” means fluorine, chlorine, bromine or iodine.

The term “aryl” when used alone or as part of a group is a 5 to 10 membered aromatic or heteroaromatic group including a phenyl group, a 5 or 6-membered monocyclic heteroaromatic group, each member of which may be optionally substituted with 1, 2, 3, 4 or 5 substituents (depending upon the number of available substitution positions), a naphthyl group or an 8-, 9- or 10-membered bicyclic heteroaromatic group, each member of which may be optionally substituted with 1, 2, 3, 4, 5 or 6 substituents (depending on the number of available substitution positions). Within this definition of aryl, suitable substitutions include C₁-C₆ alkyl, C₂-C₆ alkenyl, C₂-C₆ alkynyl, amino, hydroxy, halogen, —SH and CF₃.

The term “aryl C₁-C₄ alkyl” as used herein means a C₁-C₄ alkyl group substituted with an aryl. Thus the term “aryl C₁-C₄ alkyl” includes benzyl, 1-phenylethyl (α-methylbenzyl), 2-phenylethyl, 1-naphthalenemethyl or 2-naphthalenemethyl.

The term “naphthyl” includes 1-naphthyl, and 2-naphthyl. 1-naphthyl is preferred.

The term “benzyl” as used herein means a monovalent unsubstituted phenyl radical linked to the point of substitution by a —CH₂— group.

The term “5- or 6-membered monocyclic heteroaromatic group” as used herein means a monocyclic aromatic group with a total of 5 or 6 atoms in the ring wherein from 1 to 4 of those atoms are each independently selected from N, O and S. Preferred groups have 1 or 2 atoms in the ring which are each independently selected from N, O and S. Examples of 5-membered monocyclic heteroaromatic groups include pyrrolyl (also called azolyl), furanyl, thienyl, pyrazolyl (also called 1H-pyrazolyl and 1,2-diazolyl), imidazolyl, oxazolyl (also called 1,3-oxazolyl), isoxazolyl (also called 1,2-oxazolyl), thiazolyl (also called 1,3-thiazolyl), isothiazolyl (also called 1,2-thiazolyl), triazolyl, oxadiazolyl, thiadiazolyl, tetrazolyl, oxatriazolyl and thiatriazolyl. Examples of 6-membered monocyclic heteroaromatic groups include pyridinyl, pyrimidyl, pyrazinyl, pyridazinyl and triazinyl.

The term “8-, 9- or 10-membered bicyclic heteroaromatic group” as used herein means a fused bicyclic aromatic group with a total of 8, 9 or 10 atoms in the ring system wherein from 1 to 4 of those atoms are each independently selected from N, O and S. Preferred groups have from 1 to 3 atoms in the ring system which are each independently selected from N, O and S. Suitable 8-membered bicyclic heteroaromatic groups include imidazo[2,1-b][1,3]thiazolyl, thieno[3,2-b]thienyl, thieno[2,3-d][1,3]thiazolyl and thieno[2,3-d]imidazolyl. Suitable 9-membered bicyclic heteroaromatic groups include indolyl, isoindolyl, benzofuranyl (also called benzo[b]furanyl), isobenzofuranyl (also called benzo[c]furanyl), benzothienyl (also called benzo[b]thienyl), isobenzothienyl (also called benzo[c]thienyl), indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl and imidazo[1,2-a]pyridine. Suitable 10-membered bicyclic heteroaromatic groups include quinolinyl, isoquinolinyl, cinnolinyl, quinazolinyl, quinoxalinyl, 1,5-naphthyridyl, 1,6-naphthyridyl, 1,7-naphthyridyl and 1,8-naphthyridyl.

The term “C₁-C₆ alkoxy” as used herein means a monovalent unsubstituted saturated straight-chain or branched-chain hydrocarbon radical having from 1 to 6 carbon atoms linked to the point of substitution by a divalent O radical. Thus the term “C₁-C₆ alkoxy” includes, for example, methoxy, ethoxy, n-propoxy, isopropoxy, n-butoxy, isobutoxy, sec-butoxy and tert-butoxy. The C₁-C₆ alkoxy group may be optionally substituted with one or more substituents.

The term “N-terminal modification” as used herein includes the addition or attachment of amino acids or chemical groups directly to the N-terminal of a peptide and the formation of chemical groups, which incorporate the nitrogen at the N-terminal of a peptide.

In a preferred embodiment, the VPAC2 receptor peptide agonist comprises a sequence of the of Formula 10 (SEQ ID NO: 18), Formula 12 (SEQ ID NO: 20) or Formula 13 (SEQ ID NO: 21) wherein there is at least one amino acid substitution selected from:
Xaa₃ is: Glu;
Xaa₈ is: Glu;
Xaa₁₂ is: hR, Orn or Lys;
Xaa₁₄ is: Aib, Gln, Ala, Leu, Lys, Orn, Cit, or hR;
Xaa₁₅ is: Aib, or Orn;
Xaa₁₆ is: Lys;
Xaa₁₇ is: Leu, Ala, Ile, Lys, or Nle;
Xaa₂₀ is: Aib, Gln, Leu, Ala, or Val;
Xaa₂₁ is: Aib, Orn, Ala, or Gln;
Xaa₂₇ is: Orn or hR;
Xaa₂₈ is: Gln, Lys, hR, Aib, Pro, or Orn; and
Xaa₂₉ is: Orn or hR.

and a C-terminal extension wherein the N-terminus of the C-terminal extension is linked to the C-terminus of the peptide of Formula 10 (SEQ ID NO: 18), Formula 12 (SEQ ID NO: 20) or Formula 13 (SEQ ID NO: 21) and wherein the C-terminal extension comprises an amino acid sequence of Formula 7 (SEQ ID NO: 15).

It is more preferred that the VPAC2 receptor peptide agonist comprises at least two of the above amino acid substitutions.

According to another embodiment of the present invention, the VPAC2 receptor peptide agonist comprises a sequence of the Formula 10 (SEQ ID NO: 18), Formula 12 (SEQ ID NO: 20) or Formula 13 (SEQ ID NO: 21) wherein Xaa₁₄ is Leu, Xaa₁₅ is Ala, Xaa₁₆ is Lys, Xaa₁₇ is Leu, and Xaa₂₀ is Gln.

According to a preferred embodiment of the present invention, there is provided a VPAC2 receptor peptide agonist comprising an amino acid sequence of Formula 10 (SEQ ID NO: 18), Formula 12 (SEQ ID NO: 20) or Formula 13 (SEQ ID NO: 21) wherein Xaa₃ is Asp or Glu, Xaa₈ is Asp or Glu, Xaa₁₂ is Arg, hR, Lys, or Orn, Xaa₁₄ is Arg, Gln, Aib, hR, Orn, Cit, Lys, Ala, or Leu, Xaa₁₅ is Lys, Aib, or Orn, Xaa₁₆ is Gln or Lys, Xaa₁₇ is Val, Leu, Ala, Ile, Lys, or Nle, Xaa₂₀ is Lys, Val, Leu, Aib, Ala, or Gln, Xaa₂₁ is Lys, Aib, Orn, Ala, or Gln, Xaa₂₇ is Lys, Orn, or hR, Xaa₂₈ is Asn, Gln, Lys, hR, Aib, Pro, or Orn and Xaa₂₉ is Lys, Orn, hR, or absent, and a C-terminal extension comprising an amino acid sequence of Formula 7 (SEQ ID NO: 15).

It is more preferred that the C-terminal extension is selected from: GGPSSGAPPPS (SEQ ID NO: 10), GGPSSGAPPPS-NH₂ (SEQ ID NO: 11), GGPSSGAPPPC (SEQ ID NO: 22), GGPSSGAPPPC-NH₂ (SEQ ID NO: 23), GRPSSGAPPPS (SEQ ID NO: 24) and GRPSSGAPPPS-NH₂ (SEQ ID NO: 25).

According to another preferred embodiment of the present invention, there is provided a VPAC2 receptor peptide agonist comprising an amino acid sequence of Formula 10 (SEQ ID NO: 18), Formula 12 (SEQ ID NO: 20) or Formula 13 (SEQ ID NO: 21) wherein Xaa₃₀ and Xaa₃₁ are absent, and a C-terminal extension comprising an amino acid sequence of Formula 7 (SEQ ID NO: 15).

Alternatively, in yet another preferred embodiment of the present invention, the VPAC2 receptor peptide agonist comprises an amino acid sequence of Formula 10 (SEQ ID NO: 18), Formula 12 (SEQ ID NO: 20) or Formula 13 (SEQ ID NO: 21) wherein Xaa₂₉, Xaa₃₀ and Xaa₃₁ are absent, and a C-terminal extension comprising an amino acid sequence of Formula 7 (SEQ ID NO: 15).

It is more preferred that the C-terminal extension is selected from: GGPSSGAPPPS (SEQ ID NO: 10) or GGPSSGAPPPS-NH₂ (SEQ ID NO: 11).

According to another preferred embodiment of the present invention, there is provided a VPAC receptor peptide agonist comprising an amino acid sequence of Formula 10 (SEQ ID NO: 18), Formula 12 (SEQ ID NO: 20) or Formula 13 (SEQ ID NO: 21) and a C-terminal extension wherein the N-terminus of the C-terminal extension is linked to the C-terminus of the peptide of Formula 10 (SEQ ID NO: 18), Formula 12 (SEQ ID NO: 20) or Formula 13 (SEQ ID NO: 21) wherein either Xaa₁₄ or Xaa₁₅ is Aib and either Xaa₂₀ or Xaa₂₁ is Aib and wherein the C-terminal extension comprises an amino acid sequence of Formula 7 (SEQ ID NO: 15).

According to yet another preferred embodiment of the present invention, there is provided a VPAC receptor peptide agonist comprising an amino acid sequence of Formula 10 (SEQ ID NO: 18), Formula 12 (SEQ ID NO: 20) or Formula 13 (SEQ ID NO: 21) and a C-terminal extension wherein the N-terminus of the C-terminal extension is linked to the C-terminus of the peptide of Formula 10 (SEQ ID NO: 18), Formula 12 (SEQ ID NO: 20) or Formula 13 (SEQ ID NO: 21) wherein either Xaa₁₄ or Xaa₁₅ is Aib and either Xaa₂₀ or Xaa₂₁ is Aib and Xaa₂₈ is Gln and Xaa₂₉ is Lys or absent, and wherein the C-terminal extension comprises an amino acid sequence of Formula 7 (SEQ ID NO: 15).

In a further preferred embodiment of the present invention, there is provided a VPAC receptor peptide agonist comprising an amino acid sequence of Formula 10 (SEQ ID NO: 18), Formula 12 (SEQ ID NO: 20) or Formula 13 (SEQ ID NO: 21) and a C-terminal extension wherein the N-terminus of the C-terminal extension is linked to the C-terminus of the peptide of Formula 10 (SEQ ID NO: 18), Formula 12 (SEQ ID NO: 20) or Formula 13 (SEQ ID NO: 21) wherein either Xaa₁₄ or Xaa₁₅ is Aib and either Xaa₂₀ or Xaa₂₁ is Aib and Xaa₁₂ of the peptide sequence is hR or Orn, Xaa₂₇ is hR or Orn and Xaa₂₉ is hR or Orn and wherein the C-terminal extension comprises an amino acid sequence of Formula 7 (SEQ ID NO: 15).

In the above preferred embodiments of the present invention, it is especially preferred that the VPAC2 receptor peptide agonist further comprises a N-terminal modification, wherein the N-terminal modification is the addition of a group selected from: acetyl, propionyl, butyryl, pentanoyl, hexanoyl, methionine, methionine sulfoxide, 3-phenylpropionyl, phenylacetyl, benzoyl, norleucine, D-histidine, isoleucine and 3-mercaptopropionyl and more preferably is the addition of acetyl or hexanoyl.

In a preferred embodiment, there is provided a VPAC2 receptor peptide agonist comprising an amino acid sequence of Formula 10 (SEQ ID NO: 18), Formula 12 (SEQ ID NO: 20) or Formula 13 (SEQ ID NO: 21) wherein either Xaa₁₄ or Xaa₁₅ is Aib and either Xaa₂₀ or Xaa₂₁ is Aib, and a C-terminal extension selected from: GGPSSGAPPPS (SEQ ID NO: 10), GGPSSGAPPPS-NH₂ (SEQ ID NO: 11), GGPSSGAPPPC (SEQ ID NO: 22), GGPSSGAPPPC-NH₂ (SEQ ID NO: 23), GRPSSGAPPPS (SEQ ID NO: 24) and GRPSSGAPPPS-NH₂ (SEQ ID NO: 25) and wherein the VPAC2 receptor peptide agonist further comprises a N-terminal modification which modification is the addition of hexanoyl or acetyl.

In another preferred embodiment, there is provided a VPAC2 receptor peptide agonist comprising an amino acid sequence of Formula 10 (SEQ ID NO: 18), Formula 12 (SEQ ID NO: 20) or Formula 13 (SEQ ID NO: 21) wherein either Xaa₁₄ or Xaa₁₅ is Aib and either Xaa₂₀ or Xaa₂₁ is Aib, Xaa₂₈ is Gln and Xaa₂₉ is Lys or absent, and a C-terminal extension selected from: GGPSSGAPPPS (SEQ ID NO: 10), GGPSSGAPPPS-NH₂ (SEQ ID NO: 11), GGPSSGAPPPC (SEQ ID NO: 22), GGPSSGAPPPC-NH₂ (SEQ ID NO: 23), GRPSSGAPPPS (SEQ ID NO: 24) and GRPSSGAPPPS-NH₂ (SEQ ID NO: 25) wherein the VPAC2 receptor peptide agonist further comprises a N-terminal modification which modification is the addition of hexanoyl or acetyl.

In yet another preferred embodiment, there is provided a VPAC2 receptor peptide agonist comprising an amino acid sequence of Formula 10 (SEQ ID NO: 18), Formula 12 (SEQ ID NO: 20) or Formula 13 (SEQ ID NO: 21) wherein either Xaa₁₄ or Xaa₁₅ is Aib and either Xaa₂₀ or Xaa₂₁ is Aib, Xaa₁₂ is hR or Orn, Xaa₂₇ is hR or Orn and Xaa₂₉ is hR or Orn, and a C-terminal extension selected from: GGPSSGAPPPS (SEQ ID NO: 10), GGPSSGAPPPS-NH₂ (SEQ ID NO: 11), GGPSSGAPPPC (SEQ ID NO: 22), GGPSSGAPPPC-NH₂ (SEQ ID NO: 23), GRPSSGAPPPS (SEQ ID NO: 24) and GRPSSGAPPPS-NH₂ (SEQ ID NO: 25) wherein the VPAC2 receptor peptide agonist further comprises a N-terminal modification which modification is the addition of hexanoyl or acetyl.

A preferred alternative sequence for selective VPAC2 receptor peptide agonists of the present invention comprises an amino acid sequence of the Formula 1 (SEQ ID NO: 4), provided that if Xaa₂₉ or Xaa₃₀ of Formula 1 is absent each amino acid downstream is absent and wherein the C-terminal amino acid may be amidated.

Another alternative preferred peptide sequence for selective VPAC2 receptor peptide agonists of the present invention comprises an amino acid sequence of the Formula 2 (SEQ ID NO: 5), provided that if Xaa₂₉ or Xaa₃₀ of Formula 2 is absent each amino acid downstream is absent and wherein the C-terminal amino acid may be amidated.

Another preferred alternative peptide sequence for selective VPAC2 receptor peptide agonists of the present invention comprises an amino acid sequence of the Formula 3 (SEQ ID NO: 6), provided that if Xaa₂₉, Xaa₃₀, Xaa₃₁, Xaa₃₂, Xaa₃₃, Xaa₃₄, Xaa₃₅, Xaa₃₆, Xaa₃₇, Xaa₃₈, or Xaa₃₉ of Formula 3 is absent, the next amino acid present downstream is the next amino acid in the sequence and wherein the C-terminal amino acid may be amidated.

Preferable alternative peptide sequences for selective VPAC2 receptor peptide agonists include:

| | |
|---|---|
| SEQ ID NO: 288 | HSDAVFTDNYTRLRKQMAVKKYLNSIKK-NH₂ |
| SEQ ID NO: 289 | HSDAVFTDNYTRLRKQMAVKKYLNSIKKGGT |
| SEQ ID NO: 290 | HSDAVFTENYTKLRKQLAAKKYLNDLLNGGT |
| SEQ ID NO: 291 | HSDAVFTDNYTKLRKQLAAKKYLNDILNGGT |
| SEQ ID NO: 292 | HSDAVFTENYTKLRKQLAAKKYLNDLKKGGTSWCEP |
| | GWCR |
| SEQ ID NO: 293 | HSDAVFTDNYTRLRKQLAAKKYLNSIKKGGT |
| SEQ ID NO: 294 | HSDAVFTDNYTRLRKQLAAKKYLNDIKNGGT |
| SEQ ID NO: 295 | HSDAVFTDNYTRLRKQLAVKKYLNSIKKGGT |
| SEQ ID NO: 296 | HSDAVFTDNYTRLRKQMAAKKYLNSIKKGGT |
| SEQ ID NO: 297 | HSDAVFTDNYTRLRKQLAVKKYLNDIKNGGT |
| SEQ ID NO: 298 | HSDAVFTDNYTRLRKQLAAKKYLNSIKNGGT |
| SEQ ID NO: 299 | HSDAVFTDNYTRLRKQLAAKKYLNDIKKKRY |
| SEQ ID NO: 300 | HSDAVFTDNYTRLRKQMAVKKYLNSIKK |
| SEQ ID NO: 301 | HSDAVFTDNYTRLRKQMAVKKYLNSIKN |
| SEQ ID NO: 302 | HSDAVFTDNYTRLRKQMAVKKYLNSILK |
| SEQ ID NO: 303 | HSDAVFTDNYTELRKQMAVKKYLNSILN |
| SEQ ID NO: 304 | HSDAVFTDNYTRLRKQMAVKKYLNDILN |
| SEQ ID NO: 305 | HSDAVFTDNYTRLRKQMAAKKYLNSIKN |
| SEQ ID NO: 306 | HSDAVFTDNYTRLRKQMAAKKYLNSILK |
| SEQ ID NO: 307 | HSDAVFTDNYTRLRKQMAAKKYLNSIKK |
| SEQ ID NO: 308 | HSDAVFTDNYTRLRKQMAAKKYLNSIKKKRY |
| SEQ ID NO: 309 | HSDAVFTDNYTRLRKQMAAKKYLNSIKKKR |
| SEQ ID NO: 310 | HSDAVFTDNYTRLRKQMAAKKYLNSIKKK |
| SEQ ID NO: 311 | HSDAVFTDNYTRLRKQMAAKKYLNSIKNKRY |
| SEQ ID NO: 312 | HSDAVFTDNYTRLRKQMAVKKYLNSIKKKRY |
| SEQ ID NO: 313 | HSDAVFTDNYTRLRKQMAVKKYLNSIKKKR |
| SEQ ID NO: 314 | HSDAVFTDNYTRLRKQMAVKKYLNSIKKK |
| SEQ ID NO: 315 | HSDAVFTDNYTRLRKQMAVKKYLNSIKNKRY |
| SEQ ID NO: 316 | HSDAVFTDNYTRLRKQVAAKKYLQSIKK |
| SEQ ID NO: 317 | HSDAVFTDNYTRLRKQIAAKKYLQTIKK |
| SEQ ID NO: 318 | HSDAVFTENYTRLRKQMAVKKYLNSLKK-NH₂ |
| SEQ ID NO: 319 | HSDAVFTDNYTRLRKQLAAKKYLNDILKGGT |
| SEQ ID NO: 320 | HSDAVFTDNYTRLRKQLAAKKYLNDILNGGT |
| SEQ ID NO: 321 | HSDAVFTDNYTRLRKQLAVKKYLNDILKGGT |
| SEQ ID NO: 322 | HSDAVFTDNYTRLRKQVAAKKYLNSIKK |
| SEQ ID NO: 323 | HSDAVFTDNYTRLRKQMAAKKYLNSIKNKR |
| SEQ ID NO: 324 | HSDAVFTDNYTRLRKQVAAKKYLQSIKNKRY |
| SEQ ID NO: 325 | HSDAVFTDNYTRLRKQLAAKKYLNTIKNKRY |
| SEQ ID NO: 326 | HSDAVFTDNYTRLRKQVAAKKYLNSIKNKRY |
| SEQ ID NO: 327 | HSDAVFTDNYTRLRKQMAAKKYLQSIKNKRY |
| SEQ ID NO: 328 | HSDAVFTDNYTRLRKQMAAKKYLNTIKNKRY |
| SEQ ID NO: 329 | HSDAVFTDQYTRLRKQMAAKKYLNSIKNKRY |
| SEQ ID NO: 330 | HSDAVFTDQYTRLRKQLAAKKYLNTIKNKRY |
| SEQ ID NO: 331 | HSDAVTDNYTRLRKQMAAHKYLNSIKNKRY |
| SEQ ID NO: 332 | HSDAVFTDNYTRLRKQMAAKHYLNSIKNKRY |
| SEQ ID NO: 333 | HSDAVFTDQYTRLRKQLAAHKYLNTIKNKRY |
| SEQ ID NO: 334 | HSDAVFTDQYTRLRKQLAAKHYLNTIKNKRY |
| SEQ ID NO: 335 | HSDAVFTDNYTRLRKQVAAKKYLQSIKKKR |
| SEQ ID NO: 336 | HSDAVFTDNYTRLRKQVAAKKYLNSIKKKR |
| SEQ ID NO: 337 | HSDAVFTDNYTRLRKQVAAKKYLNSIKNKRY |
| SEQ ID NO: 338 | HSDAVFTDNYTRLRKQVAVKKYLQSIKKKR |
| SEQ ID NO: 339 | HSDAVFTDNYTRLRKQVAVKKYLQSIKKK |
| SEQ ID NO: 340 | HSDAVFTDNYTRLRKQVAVKKYLQSIKNKRY |
| SEQ ID NO: 341 | HSDAVFTDNYTRLRKQVAAKKYLQSILKKRY |
| SEQ ID NO: 342 | HSDAVFTDNYTRLRKQVAAKKYLQSILKKR |
| SEQ ID NO: 343 | HSDAVFTDNYTRLRKQVAAKKYLQSILKK |
| SEQ ID NO: 344 | HSDAVFTDNYTRLRKQVAAKKYLQSIKNK |
| SEQ ID NO: 345 | HSDAVFTDNYTRLRKQVAVKKYLQSILKKRY |
| SEQ ID NO: 346 | HSDAVFTDNYTRLRKQVAVKKYLQSILKKR |
| SEQ ID NO: 347 | HSDAVFTDNYTRLRKQVAVKKYLQSILKK |
| SEQ ID NO: 348 | HSDAVFTDNYTRLRKQVAVKKYLQSIKNK |
| SEQ ID NO: 349 | HSDAVFTDNYTRLRKQVAAKKYLQSILNKRY |
| SEQ ID NO: 350 | HSDAVFTDNYTRLRKQVAAKKYLQSILNKR |
| SEQ ID NO: 351 | HSDAVFTDNYTRLRKQVAAKKYLQSILNK |
| SEQ ID NO: 352 | HSDAVFTDNYTRLRKQMAEKKYLNSIKNKR |
| SEQ ID NO: 353 | HSDAVFTDNYTRLRKQMAFKKYLNSIKNKR |
| SEQ ID NO: 354 | HSDAVFTDNYTRLRKQMAGKKYLNSIKNKR |
| SEQ ID NO: 355 | HSDAVFTDNYTRLRKQMAHKKYLNSIKNKR |
| SEQ ID NO: 356 | HSDAVFTDNYTRLRKQMAIKKYLNSIKNKR |
| SEQ ID NO: 357 | HSDAVFTDNYTRLRKQMAKKKYLNSIKNKR |
| SEQ ID NO: 358 | HSDAVFTDNYTRLRKQMALKKYLNSIKNKR |
| SEQ ID NO: 359 | HSDAVFTDNYTRLRKQMAMKKYLNSIKNKR |
| SEQ ID NO: 360 | HSDAVFTDNYTRLRKQMANKKYLNSIKNKR |
| SEQ ID NO: 361 | HSDAVFTDNYTRLRKQMAPKKYLNSIKNKR |
| SEQ ID NO: 362 | HSDAVFTDNYTRLRKQMAQKKYLNSIKNKR |
| SEQ ID NO: 363 | HSDAVFTDNYTRLRKQMARKKYLNSIKNKR |
| SEQ ID NO: 364 | HSDAVFTDNYTRLRKQMASKKYLNSIKNKR |
| SEQ ID NO: 365 | HSDAVFTDNYTRLRKQMATKKYLNSIKNKR |
| SEQ ID NO: 366 | HSDAVFTDNYTRLRKQMAVKKYLNSIKNKR |
| SEQ ID NO: 367 | HSDAVFTDNYTRLRKQMAWKKYLNSIKNKR |
| SEQ ID NO: 368 | HSDAVFTDNYTRLRKQMAYKKYLNSIKNKR |
| SEQ ID NO: 369 | HSDAVFTDNYTRLRKQMAAKKYLNSIANKR |
| SEQ ID NO: 370 | HSDAVFTDNYTRLRKQMAAKKYLNSIDNKR |
| SEQ ID NO: 371 | HSDAVFTDNYTRLRKQMAAKKYLNSIENKR |
| SEQ ID NO: 372 | HSDAVFTDNYTRLRKQMAAKKYLNSIFNKR |
| SEQ ID NO: 373 | HSDAVFTDNYTRLRKQMAAKKYLNSIGNKR |
| SEQ ID NO: 374 | HSDAVFTDNYTRLRKQMAAKKYLNSIHNKR |
| SEQ ID NO: 375 | HSDAVFTDNYTRLRKQMAAKKYLNSIINKR |
| SEQ ID NO: 376 | HSDAVFTDNYTRLRKQMAAKKYLNSIMNKR |
| SEQ ID NO: 377 | HSDAVFTDNYTRLRKQMAAKKYLNSINNKR |
| SEQ ID NO: 378 | HSDAVFTDNYTRLRKQMAAKKYLNSIPNKR |
| SEQ ID NO: 379 | HSDAVFTDNYTRLRKQMAAKKYLNSIQNKR |
| SEQ ID NO: 380 | HSDAVFTDNYTRLRKQMAAKKYLNSIRNKR |
| SEQ ID NO: 381 | HSDAVFTDNYTRLRKQMAAKKYLNSISNKR |
| SEQ ID NO: 382 | HSDAVFTDNYTRLRKQMAAKKYLNSITNKR |
| SEQ ID NO: 383 | HSDAVFTDNYTRLRKQMAAKKYLNSIVNKR |
| SEQ ID NO: 384 | HSDAVFTDNYTRLRKQMAAKKYLNSIWNKR |
| SEQ ID NO: 385 | HSDAVFTDNYTRLRKQMAAKKYLNSIYNKR |
| SEQ ID NO: 386 | HSDAVFTDNYTRLRKQMAAKKYLNSIKNAR |
| SEQ ID NO: 387 | HSDAVFTDNYTRLRKQMAAKKYLNSIKNDR |
| SEQ ID NO: 388 | HSDAVFTDNYTRLRKQMAAKKYLNSIKNER |
| SEQ ID NO: 389 | HSDAVFTDNYTRLRKQMAAKKYLNSIKNFR |
| SEQ ID NO: 390 | HSDAVFTDNYTRLRKQMAAKKYLNSIKNGR |
| SEQ ID NO: 391 | HSDAVFTDNYTRLRKQMAAKKYLNSIKNHR |
| SEQ ID NO: 392 | HSDAVFTDNYTRLRKQMAAKKYLNSIKNIR |
| SEQ ID NO: 393 | HSDAVFTDNYTRLRKQMAAKKYLNSIKNLR |
| SEQ ID NO: 394 | HSDAVFTDNYTRLRKQMAAKKYLNSIKNMR |
| SEQ ID NO: 395 | HSDAVFTDNYTRLRKQMAAKKYLNSIKNNR |
| SEQ ID NO: 396 | HSDAVFTDNYTRLRKQMAAKKYLNSIKNPR |
| SEQ ID NO: 397 | HSDAVFTDNYTRLRKQMAAKKYLNSIKNQR |
| SEQ ID NO: 398 | HSDAVFTDNYTRLRKQMAAKKYLNSIKNRR |
| SEQ ID NO: 399 | HSDAVFTDNYTRLRKQMAAKKYLNSIKNSR |
| SEQ ID NO: 400 | HSDAVFTDNYTRLRKQMAAKKYLNSIKNTR |
| SEQ ID NO: 401 | HSDAVFTDNYTRLRKQMAAKKYLNSIKNVR |
| SEQ ID NO: 402 | HSDAVFTDNYTRLRKQMAAKKYLNSIKNWR |
| SEQ ID NO: 403 | HSDAVFTDNYTRLRKQMAAKKYLNSIKNYR |
| SEQ ID NO: 404 | HSDAVFTDNYTRLRKQMAAKKYLNSIKNKA |
| SEQ ID NO: 405 | HSDAVFTDNYTRLRKQMAAKKYLNSIKNKD |
| SEQ ID NO: 406 | HSDAVFTDNYTRLRKQMAAKKYLNSIKNKE |
| SEQ ID NO: 407 | HSDAVFTDNYTRLRKQMAAKKYLNSIKNKF |
| SEQ ID NO: 408 | HSDAVFTDNYTRLRKQMAAKKYLNSIKNKG |
| SEQ ID NO: 409 | HSDAVFTDNYTRLRKQMAAKKYLNSIKNKH |
| SEQ ID NO: 410 | HSDAVFTDNYTRLRKQMAAKKYLNSIKNKI |
| SEQ ID NO: 411 | HSDAVFTDNYTRLRKQMAAKKYLNSIKNKK |
| SEQ ID NO: 412 | HSDAVFTDNYTRLRKQMAAKKYLNSIKNKL |
| SEQ ID NO: 413 | HSDAVFTDNYTRLRKQMAAKKYLNSIKNKM |
| SEQ ID NO: 414 | HSDAVFTDNYTRLRKQMAAKKYLNSIKNKN |
| SEQ ID NO: 415 | HSDAVFTDNYTRLRKQMAAKKYLNSIKNKP |
| SEQ ID NO: 416 | HSDAVFTDNYTRLRKQMAAKKYLNSIKNKQ |
| SEQ ID NO: 417 | HSDAVFTDNYTRLRKQMAAKKYLNSIKNKS |
| SEQ ID NO: 418 | HSDAVFTDNYTRLRKQMAAKKYLNSIKNKT |
| SEQ ID NO: 419 | HSDAVFTDNYTRLRKQMAAKKYLNSIKNKV |
| SEQ ID NO: 420 | HSDAVFTDNYTRLRKQMAAKKYLNSIKNKW |
| SEQ ID NO: 421 | HSDAVFTDNYTRLRKQMAAKKYLNSIKNKY |
| SEQ ID NO: 422 | HSDAVFTDNYTRLRKQVAAKKYLQSIKNKRYSWCEP |
| | GWCR |
| SEQ ID NO: 423 | HSDAVFTDDYTRLRKEVAAKKYLESIKDKRY |
| SEQ ID NO: 424 | HSDAVFTDNYTRLRKQMAAKKYLNSIKNRI |
| SEQ ID NO: 425 | HSDAVFTDNYTRLRKQMAGKKYLNSIKNRI |
| SEQ ID NO: 426 | HSDAVFTDNYTRLRKQMAKKKYLNSIKNRI |
| SEQ ID NO: 427 | HSDAVFTDNYTRLRKQMARKKYLNSIKNRI |
| SEQ ID NO: 428 | HSDAVFTDNYTRLRKQMASKKYLNSIKNRI |
| SEQ ID NO: 429 | HSDAVFTDNYTRLRKQMAAKKYLNSIPNRI |
| SEQ ID NO: 430 | HSDAVFTDNYTRLRKQMAGKKYLNSIPNRI |
| SEQ ID NO: 431 | HSDAVFTDNYTRLRKQMAKKKYLNSIPNRI |
| SEQ ID NO: 432 | HSDAVFTDNYTRLRKQMARKKYLNSIPNRI |
| SEQ ID NO: 433 | HSDAVFTDNYTRLRKQMASKKYLNSIPNRI |
| SEQ ID NO: 434 | HSDAVFTDNYTRLRKQMAAKKYLNSIQNRI |
| SEQ ID NO: 435 | HSDAVFTDNYTRLRKQMAGKKYLNSIQNRI |
| SEQ ID NO: 436 | HSDAVFTDNYTRLRKQMAKKKYLNSIQNRI |
| SEQ ID NO: 437 | HSDAVFTDNYTRLRKQMARKKYLNSIQNRI |
| SEQ ID NO: 438 | HSDAVFTDNYTRLRKQMASKKYLNSIQNRI |
| SEQ ID NO: 439 | HSDAVFTDNYTRLRKQMAAKKYLNSIRNRI |
| SEQ ID NO: 440 | HSDAVFTDNYTRLRKQMAGKKYLNSIRNRI |
| SEQ ID NO: 441 | HSDAVFTDNYTRLRKQMAKKKYLNSIRNRI |
| SEQ ID NO: 442 | HSDAVFTDNYTRLRKQMARKKYLNSIRNRI |
| SEQ ID NO: 443 | HSDAVFTDNYTRLRKQMASKKYLNSIRNRI |
| SEQ ID NO: 444 | HSDAVFTENYTKLRKQLAAKKYLNDLKKGGT-NH2 |
| SEQ ID NO: 445 | HSDAVFTENYTKLRKQLAAKKYLNDLKKGGT |
| SEQ ID NO: 446 | HSDAVFTENYTKLRKQLAAKKYLNDLKKGGT |
| SEQ ID NO: 447 | HSDAVFTENYTKLRKQLAAKKYLNDLKK |
| SEQ ID NO: 448 | HSDAVFTDNYTRLRKQLAAKKYLNDIKKGGT |
| SEQ ID NO: 449 | HSDAVFTDNYTRLRKQLAAKKYLNDIKK-NH2 |
| SEQ ID NO: 450 | HSDAVFTDNYTRLRKQMAVKKYLNDLKKGGT |
| SEQ ID NO: 451 | HSDAVFTDNYTRLRKQMAAKKYLNDIKKGGT |
| SEQ ID NO: 452 | HSDAVFTDNYTRLRKQLAVKKYLNDIKKGGT |
| SEQ ID NO: 453 | HSDAVFTDNYTRLRKQLAAKKYLNDIKKGG |
| SEQ ID NO: 454 | HSDAVFTDNYTRLRKQLAAKKYLNDIKKG |
| SEQ ID NO: 455 | HSDAVFTDNYTRLRKQLAAKKYLNDIKK |
| SEQ ID NO: 456 | HSDAVFTDNYTRLRKQLAAKKYLNDIKKQ |
| SEQ ID NO: 457 | HSDAVFTDNYTRLRKQLAAKKYLNDIKKNQ |
| SEQ ID NO: 458 | HSDAVFTDNYTRLREQMAVKKYLNSILN |
| SEQ ID NO: 459 | HSDAVFTDNYTRLRKQLAVKKYLNSILN |
| SEQ ID NO: 460 | HSDAVFTDNYTRLRKQMAAKKYLNSILN |
| SEQ ID NO: 461 | HSDAVFTENYTKLRKQLAAKKYLNDLKKGGT |
| SEQ ID NO: 462 | HSDAVFTDNYTRLRKQMACKKYLNSIKNKR |
| SEQ ID NO: 463 | HSDAVFTDNYTRLRKQMADKKYLNSIKNKR |
| SEQ ID NO: 464 | HSDAVFTDNYTRLRKQMAAKKYLNSICNKR |
| SEQ ID NO: 465 | HSDAVFTDNYTRLRKQMAAKKYLNSIKNCR |
| SEQ ID NO: 466 | HSDAVFTDQYTRLRKQVAAKKYLQSIKQKRY |
| SEQ ID NO: 467 | HTDAVFTDQYTRLRKQVAAKKYLQSIKQKRY |
| SEQ ID NO: 468 | HSDAVFTDQYTRLRKQMAAKKYLQSIKQKRY |
| SEQ ID NO: 469 | HSDAVFTDQYTRLRKQVAAKKYLQSIKQK |
| SEQ ID NO: 470 | HTEAVFTDQYTRLRKQVAAKKYLQSIKQKRY |
| SEQ ID NO: 471 | HSDAVFTDQYTRLRKQLAVKKYLQDIKQGGT |
| SEQ ID NO: 472 | HSDAVFTDQYTRLRKQMAAKKYLQSIKQKR |
| SEQ ID NO: 473 | HSDAVFTDQYTRLRKQLAAKKYLQTIKQKRY |
| SEQ ID NO: 474 | HSDAVFTDQYTRLRKQMAAKKYLQTIKQKRY |
| SEQ ID NO: 475 | HSDAVFTDQYTRLRKQMAAHKYLQSIKQKRY |
| SEQ ID NO: 476 | HSDAVFTDQYTRLRKQMAAKKYLQSIKQKRY |
| SEQ ID NO: 477 | HSDAVFTDQYTRLRKQMAGKKYLQSIKQKR |
| SEQ ID NO: 478 | HSDAVFTDQYTRLRKQMAKKKYLQSIKQKR |
| SEQ ID NO: 479 | HSDAVFTDQYTRLRKQMARKKYLQSIKQKR |
| SEQ ID NO: 480 | HSDAVFTDQYTRLRKQMASKKYLQSIKQKR |
| SEQ ID NO: 481 | HSDAVFTDQYTRLRKQMAAKKYLQSIPQKR |
| SEQ ID NO: 482 | HSDAVFTDQYTRLRKQMAAKKYLQSIQQKR |
| SEQ ID NO: 483 | HSDAVFTDQYTRLRKQMAAKKYLQSIRQKR |
| SEQ ID NO: 484 | HSDAVFTDQYTRLRKQMAAKKYLQSIKQRR |
| SEQ ID NO: 485 | HSDAVFTDQYTRLRKQMAAKKYLQSIKQKA |
| SEQ ID NO: 486 | HSDAVFTDQYTRLRKQMAAKKYLQSIKQKF |
| SEQ ID NO: 487 | HSDAVFTDQYTRLRKQMAAKKYLQSIKQKH |
| SEQ ID NO: 488 | HSDAVFTDQYTRLRKQMAAKKYLQSIKQKI |
| SEQ ID NO: 489 | HSDAVFTDQYTRLRKQMAAKKYLQSIKQKK |
| SEQ ID NO: 490 | HSDAVFTDQYTRLRKQMAAKKYLQSIKQKL |
| SEQ ID NO: 491 | HSDAVFTDQYTRLRKQMAAKKYLQSIKQKM |
| SEQ ID NO: 492 | HSDAVFTDQYTRLRKQMAAKKYLQSIKQKP |
| SEQ ID NO: 493 | HSDAVFTDQYTRLRKQMAAKKYLQSIKQKQ |
| SEQ ID NO: 494 | HSDAVFTDQYTRLRKQMAAKKYLQSIKQKS |
| SEQ ID NO: 495 | HSDAVFTDQYTRLRKQMAAKKYLQSIKQKT |
| SEQ ID NO: 496 | HSDAVFTDQYTRLRKQMAAKKYLQSIKQKV |
| SEQ ID NO: 497 | HSDAVFTDQYTRLRKQMAAKKYLQSIKQKW |
| SEQ ID NO: 498 | HSDAVFTDQYTRLRKQMAAKKYLQSIKQKY |
| SEQ ID NO: 499 | HSDAVFTDQYTRLRKQMAGKKYLQSIKQRI |
| SEQ ID NO: 500 | HSDAVFTDQYTRLRKQMAKKKYLQSIKQRI |
| SEQ ID NO: 501 | HSDAVFTDQYTRLRKQMASKKYLQSIKQRI |
| SEQ ID NO: 502 | HSDAVFTDQYTRLRKQMAAKKYLQSIPQRI |
| SEQ ID NO: 503 | HSDAVFTDQYTRLRKQMASKKYLQSIRQRI |
| SEQ ID NO: 504 | HSDAVFTDNYTRLRKQVAAKKYLQSIKQKRY |
| SEQ ID NO: 505 | HTDAVFTDNYTRLRKQVAAKKYLQSIKQKRY |
| SEQ ID NO: 506 | HSDAVFTDNYTRLRKQMAAKKYLQSIKQKRY |
| SEQ ID NO: 507 | HSDAVFTDNYTRLRKQVAAKKYLQSIKQK |
| SEQ ID NO: 508 | HTEAVFTDNYTRLRKQVAAKKYLQSIKQKRY |
| SEQ ID NO: 509 | HSDAVFTDNYTRLRKQLAVKKYLQDIKQGGT |
| SEQ ID NO: 510 | HSDAVFTDNYTRLRKQMAAKKYLQSIKQKR |
| SEQ ID NO: 511 | HSDAVFTDNYTRLRKQLAAKKYLQTIKQKRY |
| SEQ ID NO: 512 | HSDAVFTDNYTRLRKQMAAKKYLQTIKQKRY |
| SEQ ID NO: 513 | HSDAVFTDNYTRLRKQMAAHKYLQSIKQKRY |
| SEQ ID NO: 514 | HSDAVFTDNYTRLRKQMAAKHYLQSIKQKRY |
| SEQ ID NO: 515 | HSDAVFTDNYTRLRKQMAGKKYLQSIKQKR |
| SEQ ID NO: 516 | HSDAVFTDNYTRLRKQMAKKKYLQSIKQKR |
| SEQ ID NO: 517 | HSDAVFTDNYTRLRKQMARKKYLQSIKQKR |
| SEQ ID NO: 518 | HSDAVFTDNYTRLRKQMASKKYLQSIKQKR |
| SEQ ID NO: 519 | HSDAVFTDNYTRLRKQMAAKKYLQSIPQKR |
| SEQ ID NO: 520 | HSDAVFTDNYTRLRKQMAAKKYLQSIQQKR |
| SEQ ID NO: 521 | HSDAVFTDNYTRLRKQMAAKKYLQSIRQKR |
| SEQ ID NO: 522 | HSDAVFTDNYTRLRKQMAAKKYLQSIKQRR |
| SEQ ID NO: 523 | HSDAVFTDNYTRLRKQMAAKKYLQSIKQKA |
| SEQ ID NO: 524 | HSDAVFTDNYTRLRKQMAAKKYLQSIKQKF |
| SEQ ID NO: 525 | HSDAVFTDNYTRLRKQMAAKKYLQSIKQKH |
| SEQ ID NO: 526 | HSDAVFTDNYTRLRKQMAAKKYLQSIKQKI |
| SEQ ID NO: 527 | HSDAVFTDNYTRLRKQMAAKKYLQSIKQKK |
| SEQ ID NO: 528 | HSDAVFTDNYTRLRKQMAAKKYLQSIKQKL |
| SEQ ID NO: 529 | HSDAVFTDNYTRLRKQMAAKKYLQSIKQKM |
| SEQ ID NO: 530 | HSDAVFTDNYTRLRKQMAAKKYLQSIKQKP |
| SEQ ID NO: 531 | HSDAVFTDNYTRLRKQMAAKKYLQSIKQKQ |
| SEQ ID NO: 532 | HSDAVFTDNYTRLRKQMAAKKYLQSIKQKS |
| SEQ ID NO: 533 | HSDAVFTDNYTRLRKQMAAKKYLQSIKQKT |
| SEQ ID NO: 534 | HSDAVFTDNYTRLRKQMAAKKYLQSIKQKV |
| SEQ ID NO: 535 | HSDAVFTDNYTRLRKQMAAKKYLQSIKQKW |
| SEQ ID NO: 536 | HSDAVFTDNYTRLRKQMAAKKYLQSIKQKY |
| SEQ ID NO: 537 | HSDAVFTDNYTRLRKQMAGKKYLQSIKQRI |
| SEQ ID NO: 538 | HSDAVFTDNYTRLRKQMAKKKYLQSIKQRI |
| SEQ ID NO: 539 | HSDAVFTDNYTRLRKQMASKKYLQSIKQRI |
| SEQ ID NO: 540 | HSDAVFTDNYTRLRKQMAAKKYLQSIPQRI |
| SEQ ID NO: 541 | HSDAVFTDNYTRLRKQMASKKYLQSIRQRI |
| SEQ ID NO: 542 | HSDAVFTDQYTRLRKQVAAKKYLQSIKNKRY |
| SEQ ID NO: 543 | HTDAVFTDQYTRLRKQVAAKKYLQSIKNKRY |
| SEQ ID NO: 544 | HSDAVFTDQYTRLRKQMAAKKYLQSIKNKRY |
| SEQ ID NO: 545 | HSDAVFTDQYTRLRKQVAAKKYLQSIKNK |
| SEQ ID NO: 546 | HTEAVFTDQYTRLRKQVAAKKYLQSIKNKRY |
| SEQ ID NO: 547 | HSDAVFTDQYTRLRKQLAVKKYLQDIKNGGT |
| SEQ ID NO: 548 | HSDAVFTDQYTRLRKQMAAKKYLQSIKNKR |
| SEQ ID NO: 549 | HSDAVFTDQYTRLRKQLAAKKYLQTIKNKRY |
| SEQ ID NO: 550 | HSDAVFTDQYTRLRKQMAAKKYLQTIKNKRY |
| SEQ ID NO: 551 | HSDAVFTDQYTRLRKQMAAHKYLQSIKNKRY |
| SEQ ID NO: 552 | HSDAVFTDQYTRLRKQMAAKHYLQSIKNKRY |
| SEQ ID NO: 553 | HSDAVFTDQYTRLRKQMAGKKYLQSIKNKR |
| SEQ ID NO: 554 | HSDAVFTDQYTRLRKQMAKKKYLQSIKNKR |
| SEQ ID NO: 555 | HSDAVFTDQYTRLRKQMARKKYLQSIKNKR |
| SEQ ID NO: 556 | HSDAVFTDQYTRLRKQMASKKYLQSIKNKR |
| SEQ ID NO: 557 | HSDAVFTDQYTRLRKQMAAKKYLQSIPNKR |
| SEQ ID NO: 558 | HSDAVFTDQYTRLRKQMAAKKYLQSIQNKR |
| SEQ ID NO: 559 | HSDAVFTDQYTRLRKQMAAKKYLQSIRNKR |
| SEQ ID NO: 560 | HSDAVFTDQYTRLRKQMAAKKYLQSIKNRR |
| SEQ ID NO: 561 | HSDAVFTDQYTRLRKQMAAKKYLQSIKNKA |
| SEQ ID NO: 562 | HSDAVFTDQYTRLRKQMAAKKYLQSIKNKF |
| SEQ ID NO: 563 | HSDAVFTDQYTRLRKQMAAKKYLQSIKNKH |
| SEQ ID NO: 564 | HSDAVFTDQYTRLRKQMAAKKYLQSIKNKI |
| SEQ ID NO: 565 | HSDAVFTDQYTRLRKQMAAKKYLQSIKNKK |
| SEQ ID NO: 566 | HSDAVFTDQYTRLRKQMAAKKYLQSIKNKL |
| SEQ ID NO: 567 | HSDAVFTDQYTRLRKQMAAKKYLQSIKNKM |
| SEQ ID NO: 568 | HSDAVFTDQYTRLRKQMAAKKYLQSIKNKP |
| SEQ ID NO: 569 | HSDAVFTDQYTRLRKQMAAKKYLQSIKNKQ |
| SEQ ID NO: 570 | HSDAVFTDQYTRLRKQMAAKKYLQSIKNKS |
| SEQ ID NO: 571 | HSDAVFTDQYTRLRKQMAAKKYLQSIKNKT |
| SEQ ID NO: 572 | HSDAVFTDQYTRLRKQMAAKKYLQSIKNKV |
| SEQ ID NO: 573 | HSDAVFTDQYTRLRKQMAAKKYLQSIKNKW |
| SEQ ID NO: 574 | HSDAVFTDQYTRLRKQMAAKKYLQSIKNKY |
| SEQ ID NO: 575 | HSDAVFTDQYTRLRKQMAGKKYLQSIKNRI |
| SEQ ID NO: 576 | HSDAVFTDQYTRLRKQMAKKKYLQSIKNRI |
| SEQ ID NO: 577 | HSDAVFTDQYTRLRKQMASKKYLQSIKNRI |
| SEQ ID NO: 578 | HSDAVFTDQYTRLRKQMAAKKYLQSIPNRI |
| SEQ ID NO: 579 | HSDAVFTDQYTRLRKQMASKKYLQSIRNRI |
| SEQ ID NO: 580 | HSDAVFTDQYTRLRKQVAAKKYLQSIKQKRYC |
| SEQ ID NO: 581 | HTDAVFTDQYTRLRKQVAAKKYLQSIKQKRYC |
| SEQ ID NO: 582 | HSDAVFTDQYTRLRKQMAAKKYLQSIKQKRYC |
| SEQ ID NO: 583 | HSDAVFTDQYTRLRKQVAAKKYLQSIKQKC |
| SEQ ID NO: 584 | HTEAVFTDQYTRLRKQVAAKKYLQSIKQKRYC |
| SEQ ID NO: 585 | HSDAVFTDQYTRLRKQLAVKKYLQDIKQGGTC |
| SEQ ID NO: 586 | HSDAVFTDQYTRLRKQMAAKKYLQSIKQKRC |
| SEQ ID NO: 587 | HSDAVFTDQYTRLRKQLAAKKYLQTIKQKRYC |
| SEQ ID NO: 588 | HSDAVFTDQYTRLRKQMAAKKYLQTIKQKRYC |
| SEQ ID NO: 589 | HSDAVFTDQYTRLRKQMAALKYLQSIKQKRYC |
| SEQ ID NO: 590 | HSDAVFTDQYTRLRKQMAAKHYLQSIKQKRYC |
| SEQ ID NO: 591 | HSDAVFTDQYTRLRKQMAGKKYLQSIKQKRC |
| SEQ ID NO: 592 | HSDAVFTDQYTRLRKQMAKKKYLQSIKQKRC |
| SEQ ID NO: 593 | HSDAVFTDQYTRLRKQMARKKYLQSIKQKRC |
| SEQ ID NO: 594 | HSDAVFTDQYTRLRKQMASKKYLQSIKQKRC |
| SEQ ID NO: 595 | HSDAVFTDQYTRLRKQMAAKKYLQSIPQKRC |
| SEQ ID NO: 596 | HSDAVFTDQYTRLRKQMAAKKYLQSIQQKRC |
| SEQ ID NO: 597 | HSDAVFTDQYTRLRKQMAAKKYLQSIRQKRC |
| SEQ ID NO: 598 | HSDAVFTDQYTRLRKQMAAKKYLQSIKQRRC |
| SEQ ID NO: 599 | HSDAVFTDQYTRLRKQMAAKKYLQSIKQKAC |
| SEQ ID NO: 600 | HSDAVFTDQYTRLRKQMAAKKYLQSIKQKFC |
| SEQ ID NO: 601 | HSDAVFTDQYTRLRKQMAAKKYLQSIKQKHC |
| SEQ ID NO: 602 | HSDAVFTDQYTRLRKQMAAKKYLQSIKQKIC |
| SEQ ID NO: 603 | HSDAVFTDQYTRLRKQMAAKKYLQSIKQKKC |
| SEQ ID NO: 604 | HSDAVFTDQYTRLRKQMAAKKYLQSIKQKLC |
| SEQ ID NO: 605 | HSDAVFTDQYTRLRKQMAAKKYLQSIKQKMC |
| SEQ ID NO: 606 | HSDAVFTDQYTRLRKQMAAKKYLQSIKQKPC |
| SEQ ID NO: 607 | HSDAVFTDQYTRLRKQMAAKKYLQSIKQKQC |
| SEQ ID NO: 608 | HSDAVFTDQYTRLRKQMAAKKYLQSIKQKSC |
| SEQ ID NO: 609 | HSDAVFTDQYTRLRKQMAAKKYLQSIKQKTC |
| SEQ ID NO: 610 | HSDAVFTDQYTRLRKQMAAKKYLQSIKQKVC |
| SEQ ID NO: 611 | HSDAVFTDQYTRLRKQMAAKKYLQSIKQKWC |
| SEQ ID NO: 612 | HSDAVFTDQYTRLRKQMAAKKYLQSIKQKYC |
| SEQ ID NO: 613 | HSDAVFTDQYTRLRKQMAGKKYLQSIKQRIC |
| SEQ ID NO: 614 | HSDAVFTDQYTRLRKQMAKKKYLQSIKQRIC |
| SEQ ID NO: 615 | HSDAVFTDQYTRLRKQMASKKYLQSIKQRIC |
| SEQ ID NO: 616 | HSDAVFTDQYTRLRKQMAAKKYLQSIPQRIC |
| SEQ ID NO: 617 | HSDAVFTDQYTRLRKQMASKKYLQSIRQRIC |

More preferably, an alternative sequence for selective VPAC2 receptor peptide agonists of the present invention comprises an amino acid sequence of the formula:

Formula 1′

(SEQ ID NO: 4′)

His-Xaa₂-Asp-Ala-Val-Phe-Thr-Asp-Asn-Tyr-Thr-
Xaa₁₂-Leu-Xaa₁₄-Xaa₁₅-Xaa₁₆-Xaa₁₇-Ala-Xaa₁₉-Xaa₂₀-
Xaa₂₁-Tyr-Leu-Xaa₂₄-Xaa₂₅-Xaa₂₆-Xaa₂₇-Asn-Xaa₂₉-
Xaa₃₀-Xaa₃₁

wherein:
Xaa₂ is: Ser, Val, or dA;
Xaa₁₂ is: Arg or Lys;
Xaa₁₄ is: Arg, Leu, or Lys;
Xaa₁₅ is: Lys, Ala, or Arg;
Xaa₁₆ is: Gln, Lys, or Ala;
Xaa₁₇ is: Met, Val, Ala, or Leu;
Xaa₁₉ is: Val, Ala or Leu;
Xaa₂₀ is: Lys, Gln, or Arg;
Xaa₂₁ is: Lys or Arg;
Xaa₂₄ is: Asn or Gln;
Xaa₂₅ is: Ser, Phe, Ile, Leu, Thr, Val, Trp, Gln, Asn, or Tyr;
Xaa₂₆ is: Ile, Leu, Thr, Val, Trp, or Tyr;
Xaa₂₇ is: Leu, hR, Arg, or Lys;
Xaa₂₉ is: Lys, Ser, Arg, or absent;
Xaa₃₀ is: Arg, Lys, or absent; and
Xaa₃₁ is: Tyr, Phe, or absent

provided that if Xaa₂₉ or Xaa₃₀ is absent each amino acid downstream is absent and wherein the C-terminal amino acid may be amidated.

Another preferred alternative sequence for selective VPAC2 receptor peptide agonists of the present invention comprises an amino acid sequence of the Formula 1 (SEQ ID NO: 4), provided that at least one Xaa selected from the group consisting of: Xaa₂, Xaa₁₄, Xaa₁₅, Xaa₁₆, Xaa₁₇, Xaa₂₀, Xaa₂₅, Xaa₂₆, Xaa₂₇, and Xaa₃₁ is an amino acid that differs from the wild-type amino acid at the corresponding position in VIP (SEQ ID NO: 1), and provided that if Xaa₂₉ or Xaa₃₀ is absent each amino acid downstream is absent, and the C-terminal amino acid may be amidated. One or more of amino acids at the following positions are preferable:
Xaa₂ is: Val or dA;
Xaa₁₄ is: Leu;
Xaa₁₅ is: Ala;
Xaa₁₆ is: Lys;
Xaa₁₇ is: Ala;
Xaa₂₀ is: Gln;
Xaa₂₅ is: Phe, Ile, Leu, Val, Trp, or Tyr;
Xaa₂₆ is: Thr, Trp, or Tyr;
Xaa₂₇ is: hR; and
Xaa₃₁ is: Phe.

More preferably for the peptide agonists of Formula 1 (SEQ ID NO: 4), Xaa₁₄ is leucine when Xaa₁₅ is alanine and Xaa₁₆ is lysine. Even more preferably, Xaa₁₄ is leucine when Xaa₁₅ is alanine, Xaa₁₆ is lysine, Xaa₁₇ is leucine, and Xaa₂₀ is glutamine.

Another preferred alternative sequence for selective VPAC2 receptor peptide agonists of the present invention comprises an amino acid sequence of the formula:

Formula 1″

(SEQ ID NO: 4″)

His-Xaa₂-Asp-Ala-Val-Phe-Thr-Asp-Asn-Tyr-Thr-
Xaa₁₂-Leu-Xaa₁₄-Xaa₁₅-Xaa₁₆-Xaa₁₇-Ala-Xaa₁₉-Xaa₂₀-
Xaa₂₁-Tyr-Leu-Xaa₂₄-Xaa₂₅-Xaa₂₆-Xaa₂₇-Asn-Xaa₂₉-
Xaa₃₀-Xaa₃₁

wherein:
Xaa₂ is: Ser, Val, or dA;
Xaa₁₂ is: Arg, Lys, hR, Orn, or Lys (isopropyl);
Xaa₁₄ is: Arg, Leu, or Lys;
Xaa₁₅ is: Lys, Ala, or Arg;
Xaa₁₆ is: Gln, Lys, or Ala;
Xaa₁₇ is: Met, Val, Ala, or Leu;
Xaa₁₉ is: Val, Ala, or Leu;
Xaa₂₀ is: Lys, Gln, or Arg;
Xaa₂₁ is: Lys or Arg;
Xaa₂₄ is: Asn or Gln
Xaa₂₅ is: Ser, Phe, Ile, Leu, Val, Trp, Tyr, Thr, Gln, or Asn;
Xaa₂₆ is: Ile, Thr, Trp, Tyr, Leu, or Val;
Xaa₂₇ is: Leu, Lys, hR, or Arg; and
Xaa₂₉ is: Lys, Ser, Arg, hR, or absent; and
Xaa₃₀ is: Arg, Lys, or absent
Xaa₃₁ is: Tyr, Phe, or absent;

provided that at least one Xaa selected from the group consisting of: Xaa₂, Xaa₁₄, Xaa₁₅, Xaa₁₆, Xaa₁₇, Xaa₂₀, Xaa₂₅, Xaa₂₆, Xaa₂₇, and Xaa₃₁ is an amino acid that differs from the wild-type amino acid at the corresponding position in VIP (SEQ ID NO: 1), provided that if Xaa₂₉ or Xaa₃₀ is absent each amino acid downstream is absent, and provided that the C-terminal amino acid may be amidated. One or more of amino acids at the following positions are preferable:
Xaa₂ is: Val or dA;
Xaa₁₄ is: Leu;
Xaa₁₅ is: Ala;
Xaa₁₆ is: Lys;
Xaa₁₇ is: Ala;
Xaa₂₀ is: Gln;
Xaa₂₅ is: Phe, Ile, Leu, Val, Trp, or Tyr;
Xaa₂₆ is: Thr, Trp, or Tyr;
Xaa₂₇ is: hR; and
Xaa₃₁ is: Phe.

More preferably, for the agonist of Formula 1″ (SEQ ID NO: 4″), Xaa₁₄ is leucine when Xaa₁₅ is alanine and Xaa₁₆ is lysine. Even more preferably, Xaa₁₄ is leucine when Xaa₁₅ is alanine, Xaa₁₆ is lysine, Xaa₁₇ is leucine, and Xaa₂₀ is glutamine.

Another preferred alternative sequence for selective VPAC2 receptor peptide agonists of the present invention comprises an amino acid sequence of the Formula 4 (SEQ ID NO: 7), provided that if Xaa₂₉, Xaa₃₀, Xaa₃1, Xaa₃₂, Xaa₃₃, Xaa₃₄, Xaa₃₅, Xaa₃₆, Xaa₃₇, Xaa₃₈, or Xaa₃₉ of Formula 4 is absent, the next amino acid present downstream is the next amino acid in the sequence and wherein the C-terminal amino acid may be amidated. For example, if Xaa₂₉ is Lys and Xaa₃₀ is absent, the next amino acid bonded to Lys at position 29 is an amino acid listed for position 31 or, if position 31 is also absent, an amino acid listed for position 32 is bonded to Lys at position 29, and so forth. Additionally, for example, if Xaa₂₉ is Lys and Xaa₃₀ through Xaa₄₀ are absent, Lys may be the C-terminal amino acid and may be amidated.

Another preferred alternative sequence for selective VPAC2 receptor peptide agonists of the present invention comprises an amino acid sequence of the Formula 8 (SEQ ID NO: 16), provided that if Xaa₂₉, Xaa₃₀, Xaa₃₁, Xaa₃₂, Xaa₃₃, Xaa₃₄, Xaa₃₅, Xaa₃₆, Xaa₃₇, Xaa₃₈, or Xaa₃₉ of Formula 8 is absent, the next amino acid present downstream is the next amino acid in the sequence and wherein the C-terminal amino acid may be amidated.

Another preferred alternative sequence for selective VPAC2 receptor peptide agonists of the present invention comprises an amino acid sequence of the Formula 9 (SEQ ID NO: 17), provided that if Xaa₂₉, Xaa₃₀, Xaa₃₁, Xaa₃₂, Xaa₃₃, Xaa₃₄, Xaa₃₅, Xaa₃₆, Xaa₃₇, Xaa₃₈, or Xaa₃₉ of Formula 9 is absent, the next amino acid present downstream is the next amino acid in the sequence and wherein the C-terminal amino acid may be amidated.

Preferably, the agonists of the present invention have a selectivity ratio where the affinity for the VPAC2 receptor is at least 50 times greater than for the VPAC1 and/or for PAC1 receptors. More preferably, this affinity is at least 100 times greater for VPAC2 than for VPAC1 and/or for PAC1. Even more preferably, the affinity is at least 200 times greater for VPAC2 than for VPAC1 and/or for PAC1. Still more preferably, the affinity is at least 500 times greater for VPAC2 than for VPAC1 and/or for PAC1. Yet more preferably, the affinity is at least 1000 times greater for VPAC2 than for VPAC1 and/or for PAC1. Preferably, these agonists have a sequence identity in the range of 60% to 70%, 60% to 65%, 65% to 70%, 70% to 80%, 70% to 75%, 75% to 80%, 80% to 90%, 80% to 85%, 85% to 90%, 90% to 97%, 90% to 95%, or 95% to 97%, with VIP (SEQ ID NO: 1). More preferably, the sequence has 61%, 64%, 68%, 71%, 75%, 79%, 82%, 86%, 89%, 93%, or 96% sequence identity with VIP.

Preferably, the C-terminal extension for an alternative embodiment of the present invention comprises an amino acid sequence of the Formula 5 (SEQ ID NO: 8), provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ of Formula 5 is absent, the next amino acid present downstream is the next amino acid in the C-terminal extension and wherein the C-terminal amino acid may be amidated. For example, if Xaa₁ is Gly and Xaa₂ is absent, the next amino acid bonded to Gly at position 1 is an amino acid listed for position 3 or, if position 3 is also absent, an amino acid listed for position 4 is bonded to Gly at position 1, and so forth. Additionally, for example, if Xaa₁ is Gly and Xaa₂ through Xaa₁₃ are absent, Gly may be the C-terminal amino acid and may be amidated.

Also, the C-terminal extension for an alternative embodiment of the present invention preferably comprises an amino acid sequence of the Formula 6 (SEQ ID NO: 9), provided that if Xaa₁, Xaa₂, Xaa₃, Xaa₄, Xaa₅, Xaa₆, Xaa₇, Xaa₈, Xaa₉, or Xaa₁₀ of Formula 6 is absent, the next amino acid present downstream is the next amino acid in the sequence and wherein the C-terminal amino acid may be amidated. For example, if Xaa₁ is Gly and Xaa₂ is absent, the next amino acid bonded to Gly at position 1 is an amino acid listed for position 3 or, if position 3 is also absent, an amino acid listed for position 4 is bonded to Gly at position 1, and so forth. Additionally, for example, if Xaa₁ is Gly and Xaa₂ through Xaa₁₁ are absent, Gly may be the C-terminal amino acid and may be amidated.

More preferably, the C-terminal extension of an alternative embodiment of the present invention includes the following sequences:

| | |
|---|---|
| SEQ ID # | Sequence |

| | |
|---|---|
| SEQ ID NO: 10 | GGPSSGAPPPS |
| SEQ ID NO: 11 | GGPSSGAPPPS-NH₂ |

Preferably, the C-terminal extension differs from SEQ ID NO: 10 or SEQ ID NO: 11 by no more than eight amino acids, still preferably by no more than seven amino acids, yet still preferably by no more than six amino acids, more preferably by no more than five amino acids, even more preferably by no more than four amino acids, still more preferably by no more than three amino acids, yet more preferably by no more than two amino acids, and most preferably by no more than one amino acid.

Another alternative more preferable C-terminal extension of the present invention can also include variants of these sequences, including:

| SEQ ID # | Sequence |
|---|---|
| SEQ ID NO: 12 | GGPSSGAPPS-NH₂ |
| SEQ ID NO: 13 | GGPSSGAPPPS-OH |
| SEQ ID NO: 14 | GGPSSGAPPS |

These sequences contain the standard single letter codes for the twenty naturally occurring amino acids. SEQ ID NO: 11 and SEQ ID NO: 12 contain sequences that are amidated at the C-terminus of the sequence.

Preferably, the C-terminal extension differs from SEQ ID NO:12, or SEQ ID NO: 14 by no more than eight amino acids, still preferably by no more than seven amino acids, yet still preferably by no more than six amino acids, more preferably by no more than five amino acids, even more preferably by no more than four amino acids, still more preferably by no more than three amino acids, yet more preferably by no more than two amino acids, and most preferably by no more than one amino acid.

Another alternative preferred C-terminal extension of the present invention comprises an amino acid sequence of the Formula 7 (SEQ ID NO: 15), provided that if Xaa₁, Xaa₂, Xaa₃, Xaa₄, Xaa₅, Xaa₆, Xaa₇, Xaa₈, Xaa₉, or Xaa₁₀ of Formula 7 is absent, the next amino acid present downstream is the next amino acid in the C-terminal extension and wherein the C-terminal amino acid may be amidated. For example, if Xaa₁ is Gly and Xaa₂ is absent, the next amino acid bonded to Gly at position 1 is an amino acid listed for position 3 or, if position 3 is also absent, an amino acid listed for position 4 is bonded to Gly at position 1, and so forth. Additionally, for example, if Xaa₁ is Gly and Xaa₂ through Xaa₁₁ are absent, Gly may be the C-terminal amino acid and may be amidated.

Another alternative preferred C-terminal extension of the present invention includes (Lys)ₙ or (Glu)ₙ wherein n is the number of lysine or glutamic acid residues added to the C-terminus and wherein n can be anywhere from one to eight residues.

The present invention includes the following alternative selective VPAC2 receptor peptide agonists:

| | |
|---|---|
| Agonist # | Sequence |

| | |
|---|---|
| P226 SEQ ID | C6-HSDAVFTDNY(OMe)TRLRKQVAAKKYLQSIKNKRY |
| NO: 619 | GGPSSGAPPPS |
| P227 SEQ ID | C6-HSDAVFTDNYTRLRKQVAAKKY(OMe)LQSIKNKRY |
| NO: 620 | GGPSSGAPPPS |

“Insulinotropic activity” refers to the ability to stimulate insulin secretion in response to elevated glucose levels, thereby causing glucose uptake by cells and decreased plasma glucose levels. Insulinotropic activity can be assessed by methods known in the art, including using experiments that measure VPAC2 receptor binding activity or receptor activation (e.g. insulin secretion by insulinoma cell lines or islets, intravenous glucose tolerance test (IVGTT), intraperitoneal glucose tolerance test (IPGTT), and oral glucose tolerance test (OGTT)). Insulinotropic activity is routinely measured in humans by measuring insulin levels or C-peptide levels. Selective VPAC2 receptor peptide agonists of the present invention have insulinotropic activity.

“In vitro potency” as used herein is the measure of the ability of a peptide to activate the VPAC2 receptor in a cell-based assay. In vitro potency is expressed as the “EC₅₀” which is the effective concentration of compound that results in a 50% of maximum increase in activity in a single dose-response experiment. For the purposes of the present invention, in vitro potency is determined using two different assays: DiscoveRx and Alpha Screen. See Example 3 for further details of these assays. Whilst these assays are performed in different ways, the results demonstrate a general correlation between the two assays.

The present invention encompasses the discovery that acylation of or specific amino acids added to the N-terminus of a peptide sequence for a selective VPAC2 receptor peptide agonist provide features that may enhance potency and/or provide stability against DPP-IV cleavage.

The present invention encompasses the discovery that specific amino acids added to the C-terminus of a peptide sequence for a VPAC2 receptor peptide agonist provide features that may protect the peptide as well as may enhance activity, selectivity, and/or potency. For example, these C-terminal extensions may stabilize the helical structure of the peptide and sites within the peptide prone to enzymatic cleavage that are located near the C-terminus. Further, many of the C-terminally extended peptides disclosed herein may be more selective for the VPAC2 receptor and can be more potent than VIP, PACAP, and other known VPAC2 receptor peptide agonists. An example of a preferred C-terminal extension is the extension peptide of exendin-4 as the C-capping sequence. Exendin-4 is found in the salivary excretions from the Gila Monster, Heloderma Suspectum, (J. Biol. Chem., Vol. 267, No. 11, April 15, pp. 7402-7405, 1992).

VIP and some known VPAC2 receptor peptide agonists are susceptible to cleavage by various enzymes and, thus, have a short in vivo half-life. Five regions, identified below, correspond to the same positions in VIP (SEQ ID NO: 1), are discussed relative to the amino acid position in VIP, and are applicable to the sequences noted herein.

Region 1 contains a cleavage site at amino acid position 2 of Formula 10, 12 and 13 for the enzyme dipeptidyl-peptidase IV (DPP-IV). Cleavage of the peptide occurs between position 2 (serine) and position 3 (aspartic acid). The compounds of the present invention are stable against DPP-IV cleavage due to various substitutions at position 2 of Formula 10, 12 and 13 and/or the addition of a N-terminal modification as discussed previously. Examples of amino acids at position 2 that may improve stability against DPP-IV inactivation preferably include valine, D-alanine, or D-serine. More preferably, position 2 is valine or D-alanine. Examples of N-terminal modifications that may improve stability against DPP-IV inactivation include the addition of acetyl, propionyl, butyryl, pentanoyl, hexanoyl, methionine, methionine sulfoxide, 3-phenylpropionyl, phenylacetyl, benzoyl, norleucine, D-histidine, isoleucine and 3-mercaptopropionyl. Preferably, the N-terminal modification is the addition of acetyl or hexanoyl. For these examples and preferred examples of N-terminal modifications, preferred amino acids at position 2 include serine as well as valine, D-alanine, or D-serine, with more preference for position 2 being substituted with valine or D-alanine. Example 8 illustrates the stability of various selective VPAC2 receptor peptide agonists against DPP-IV inactivation encompassed by the present invention.

Regions 2 and 3, which encompass basic amino acids at positions 14 and 15 and positions 20 and 21 respectively in wild-type VIP as well as numerous VPAC2 receptor agonists known in the art, are also susceptible to enzymatic cleavage. The selective VPAC2 receptor agonists of the present invention generally have improved proteolytic stability in vivo due to substitutions in these two regions. These substitutions can render the peptide resistant to cleavage by trypsin-like enzymes, including trypsin. Examples of amino acids at position 14 that confer some resistance to cleavage by trypsin-like enzymes alone or in combination with the amino acids specified for position 15 below include glutamine, amino isobutyric acid, homoarginine, ornithine, citrulline, lysine, alanine and leucine. Also, position 14 may be arginine when position 15 is an amino acid other than lysine. Also, position 14 can be arginine when position 15 is lysine, but this specific combination does not address enzymatic cleavage. Examples of amino acids at position 15 that confer some resistance to cleavage by trypsin-like enzymes alone or in combination with amino acids specified above for position 14 include amino isobutyric acid and ornithine. Also, position 15 may be lysine when position 14 is an amino acid other than arginine. Also, position 15 can be lysine when position 14 is arginine, but this specific combination does not address enzymatic cleavage. Examples of amino acids at position 20 that confer some resistance to cleavage by trypsin-like enzymes alone or in combination with amino acids specified for position 21 include valine, leucine, amino isobutyric acid, alanine and glutamine. Also, position 20 may be lysine when position 21 is an amino acid other than lysine. Also, position 20 can be lysine when position 21 is lysine, but this specific combination does not address enzymatic cleavage. An example of an amino acid at position 21 that confers some resistance to cleavage by trypsin-like peptides alone or in combination with amino acids specified for position 20 include amino isobutyric acid, ornithine, alanine, or glutamine. Also, position 21 may be lysine when position 20 is an amino acid other than lysine. Also, position 21 can be lysine when position 20 is lysine, but this specific combination does not address enzymatic cleavage. The improved stability of a representative number of selective VPAC2 receptor peptide agonists with resistance to peptidase cleavage and encompassed by the present invention is demonstrated in Example 6.

Region 4 encompasses the amino acids at positions 25 and 26 of Formula 10, 12 and 13. Region 4 is another area that is susceptible to enzymatic cleavage. This cleavage site can be completely or partially eliminated through substitution of the amino acid at position 25 and/or the amino acid at position 26. Examples of amino acids at position 25 that confer at least some resistance to enzymatic cleavage include phenylalanine, isoleucine, leucine, threonine, valine, tryptophan, glutamine, asparagine, tyrosine, or amino isobutyric acid. Also, position 25 may be serine when position 26 is an amino acid other than isoleucine. Also, position 25 can be serine when position 26 is isoleucine, but this specific combination does not address enzymatic cleavage. Examples of amino acids at position 26 that confer at least some resistance to enzymatic cleavage alone or in combination with the amino acids specified above for position 25 include leucine, threonine, valine, tryptophan, tyrosine, phenylalanine, or amino isobutyric acid. Also, position 26 may be isoleucine when position 25 is an amino acid other than serine. Also, position 26 can be isoleucine when position 25 is serine, but this specific combination does not address enzymatic cleavage.

Region 4 also encompasses the amino acids at positions 27, 28, 29, 30 and 31 respectively in wild-type VIP as well as in many VPAC2 receptor peptide agonists known in the art. This area is also susceptible to enzymatic cleaving. The addition of a C-terminal extension peptide may render the peptide agonist more stable against neutroendopeptidase (NEP). The addition of the extension peptide may also increase selectivity for the VPAC2 receptor. Trypsin-like enzymes may also attack these positions. If that occurs, the peptide agonist may lose its C-terminal extension with the additional carboxypeptidase activity leading to an inactive form of the peptide.

In addition to selective VPAC2 receptor peptide agonists with resistance to cleavage by various peptidases, the selective VPAC2 peptide receptor agonists of the present invention may also encompass peptides with enhanced selectivity for the VPAC2 receptor, increased potency, and/or increased stability compared with some peptides known in the art. Examples of amino acid positions that may affect such properties include positions: 3, 8, 12, 14, 15, 16, 17, 20, 21, 27, 28, and 29 of Formula 10, 12, or 13. For example, the amino acid at position 3 is preferably aspartic acid or glutamic acid; the amino acid at position 8 is preferably aspartic acid or glutamic acid; the amino acid at position 12 is preferably arginine, homoarginine, ornithine, or lysine; the amino acid at position 14 is preferably arginine, glutamine, amino isobutyric acid, homoarginine, ornithine, citrulline, lysine, alanine, or leucine; the amino acid at position 15 is preferably lysine, amino isobutyric acid, or ornithine; the amino acid at position 16 is preferably glutamine or lysine; the amino acid at position 17 is preferably valine, alanine, leucine, isoleucine, lysine, or norleucine; the amino acid at position 20 is preferably lysine, valine, leucine, amino isobutyric acid, alanine, or glutamine; the amino acid at position 21 is preferably lysine, amino isobutyric acid, ornithine, alanine, or glutamine; the amino acid at position 27 is preferably lysine, ornithine, or homoarginine; the amino acid at position 28 is preferably asparagine, glutamine, lysine, homoarginine, amino isobutyric acid, proline, or ornithine; and, if present, the amino acid at position 29 is preferably lysine, ornithine, or homoarginine. Preferably, these amino acid substitutions may be combined with substitutions at positions that affect the five aforementioned regions susceptible to cleavage by various enzymes.

The increased potency and selectivity for various VPAC2 receptor peptide agonists of the present invention is demonstrated in Examples 3 and 4. For example, Table 1 in Example 3 provides a list of selective VPAC2 receptor peptide agonists and their corresponding in vitro potency results. Preferably, the selective VPAC2 receptor peptide agonists of the present invention have an EC₅₀ value less than 2 nM. More preferably, the EC₅₀ value is less than 1 nM. Even more preferably, the EC₅₀ value is less than 0.5 nM. Still more preferably, the EC₅₀ value is less than 0.1 nM.

Table 2 in Example 4 provides a list of VPAC2 receptor peptide agonists and their corresponding selectivity results for VPAC2, VPAC1, and PAC1. See Example 4 for further details of these assays. These results are provided as a ratio of VPAC2 binding affinity to VPAC1 binding affinity and as a ratio of VPAC2 binding affinity to PAC1 binding affinity. Preferably, the agonists of the present invention have a selectivity ratio where the affinity for the VPAC2 receptor is at least 50 times greater than for the VPAC1 and/or for PAC1 receptors. More preferably, this affinity is at least 100 times greater for VPAC2 than for VPAC1 and/or for PAC1. Even more preferably, the affinity is at least 200 times greater for VPAC2 than for VPAC1 and/or for PAC1. Still more preferably, the affinity is at least 500 times greater for VPAC2 than for VPAC1 and/or for PAC1. Yet more preferably, the ratio is at least 1000 times greater for VPAC2 than for VPAC1 and/or for PAC1.

As used herein, “selective VPAC2 receptor peptide agonists” also include pharmaceutically acceptable salts of the compounds described herein. A selective VPAC2 receptor peptide agonist of this invention can possess a sufficiently acidic, a sufficiently basic, or both functional groups, and accordingly react with any of a number of inorganic bases, and inorganic and organic acids, to form a salt. Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, trifluoroacetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

The selective VPAC2 receptor peptide agonists of the present invention can be administered parenterally. Parenteral administration can include, for example, systemic administration, such as by intramuscular, intravenous, subcutaneous, intradermal, or intraperitoneal injection. These agonists can be administered to the subject in conjunction with an acceptable pharmaceutical carrier, diluent, or excipient as part of a pharmaceutical composition for treating NIDDM. Standard pharmaceutical formulation techniques may be employed such as those described in Remington's Pharmaceutical Sciences, Mack Publishing Company, Easton, Pa. The selective VPAC2 receptor peptide agonists of the present invention may be formulated for administration through the buccal, topical, oral, transdermal, nasal, or pulmonary route.

The selective VPAC2 receptor peptide agonists described herein can be used to treat subjects with a wide variety of diseases and conditions. Agonists encompassed by the present invention exert their biological effects by acting at a receptor referred to as the VPAC2 receptor. Subjects with diseases and/or conditions that respond favorably to VPAC2 receptor stimulation or to the administration of VPAC2 receptor peptide agonists can therefore be treated with the VPAC2 agonists of the present invention. These subjects are said to “be in need of treatment with VPAC2 agonists” or “in need of VPAC2 receptor stimulation”.

The selective VPAC2 receptor peptide agonists of the present invention may be employed to treat diabetes, including both type 1 and type 2 diabetes (non-insulin dependent diabetes mellitus). Also included are subjects requiring prophylactic treatment with a VPAC2 receptor agonist, e.g., subjects at risk for developing NIDDM. Such treatment may also delay the onset of diabetes and diabetic complications. Additional subjects include those with impaired glucose tolerance or impaired fasting glucose, subjects whose body weight is about 25% above normal body weight for the subject's height and body build, subjects having one or more parents with NIDDM, subjects who have had gestational diabetes, and subjects with metabolic disorders such as those resulting from decreased endogenous insulin secretion. The selective VPAC2 receptor peptide agonists may be used to prevent subjects with impaired glucose tolerance from proceeding to develop type 2 diabetes, prevent pancreatic β-cell deterioration, induce β-cell proliferation, improve β-cell function, activate dormant β-cells, differentiate cells into β-cells, stimulate β-cell replication, and inhibit β-cell apoptosis. Other diseases and conditions that may be treated or prevented using compounds of the invention in methods of the invention include: Maturity-Onset Diabetes of the Young (MODY) (Herman, et al., Diabetes 43:40, 1994); Latent Autoimmune Diabetes Adult (LADA) (Zimmet, et al., Diabetes Med. 11:299, 1994); impaired glucose tolerance (IGT) (Expert Committee on Classification of Diabetes Mellitus, Diabetes Care 22 (Supp. 1):S5, 1999); impaired fasting glucose (IFG) (Charles, et al., Diabetes 40:796, 1991); gestational diabetes (Metzger, Diabetes, 40:197, 1991); metabolic syndrome X, dyslipidemia, hyperglycemia, hyperinsulinemia, hypertriglyceridemia, and insulin resistance.

The selective VPAC2 receptor peptide agonists of the present invention may also be effective in the prevention or treatment of such disorders as obesity, atherosclerotic disease, hyperlipidemia, hypercholesteremia, low HDL levels, hypertension, primary pulmonary hypertension, cardiovascular disease (including atherosclerosis, coronary heart disease, coronary artery disease, and hypertension), cerebrovascular disease and peripheral vessel disease; and for the treatment of lupus, polycystic ovary syndrome, carcinogenesis, and hyperplasia, asthma, male and female reproduction problems, sexual disorders, ulcers, sleep disorders, disorders of lipid and carbohydrate metabolism, circadian dysfunction, growth disorders, disorders of energy homeostasis, immune diseases including autoimmune diseases (e.g., systemic lupus erythematosus), as well as acute and chronic inflammatory diseases, rheumatoid arthritis, and septic shock.

The selective VPAC2 receptor peptide agonists of the present invention may also be useful for treating physiological disorders related to, for example, cell differentiation to produce lipid accumulating cells, regulation of insulin sensitivity and blood glucose levels, which are involved in, for example, abnormal pancreatic β-cell function, insulin secreting tumors and/or autoimmune hypoglycemia due to autoantibodies to insulin, autoantibodies to the insulin receptor, or autoantibodies that are stimulatory to pancreatic β-cells, macrophage differentiation which leads to the formation of atherosclerotic plaques, inflammatory response, carcinogenesis, hyperplasia, adipocyte gene expression, adipocyte differentiation, reduction in the pancreatic β-cell mass, insulin secretion, tissue sensitivity to insulin, liposarcoma cell growth, polycystic ovarian disease, chronic anovulation, hyperandrogenism, progesterone production, steroidogenesis, redox potential and oxidative stress in cells, nitric oxide synthase (NOS) production, increased gamma glutamyl transpeptidase, catalase, plasma triglycerides, HDL, and LDL cholesterol levels, and the like.

The selective VPAC2 receptor peptide agonists of the invention may also be used in methods of the invention to treat secondary causes of diabetes (Expert Committee on Classification of Diabetes Mellitus, Diabetes Care 22 (Supp. 1):S5, 1999). Such secondary causes include glucocorticoid excess, growth hormone excess, pheochromocytoma, and drug-induced diabetes. Drugs that may induce diabetes include, but are not limited to, pyriminil, nicotinic acid, glucocorticoids, phenyloin, thyroid hormone, β-adrenergic agents, α-interferon and drugs used to treat HIV infection.

In addition, the selective VPAC2 receptor peptide agonists of the invention may be used for treatment of asthma (Bolin, et al., Biopolymer 37:57-66, 1995; U.S. Pat. No. 5,677,419; showing that polypeptide R3PO is active in relaxing guinea pig tracheal smooth muscle); hypotension induction (VIP induces hypotension, tachycardia, and facial flushing in asthmatic patients (Morice, et al., Peptides 7:279-280, 1986; Morice, et al., Lancet 2:1225-1227, 1983); male reproduction problems (Siow, et al., Arch. Androl. 43(1):67-71, 1999); as an anti-apoptosis/neuroprotective agent (Brenneman, et al., Ann. N.Y. Acad. Sci. 865:207-12, 1998); cardioprotection during ischemic events (Kalfin, et al., J. Pharmacol. Exp. Ther. 1268(2):952-8, 1994; Das, et al., Ann. N.Y. Acad. Sci. 865:297-308, 1998), manipulation of the circadian clock and its associated disorders (Hamar, et al., Cell 109:497-508, 2002; Shen, et al., Proc. Natl. Acad. Sci. 97:11575-80, 2000), and as an anti-ulcer agent (Tuncel, et al., Ann. N.Y. Acad. Sci. 865:309-22, 1998).

An “effective amount” of a selective VPAC2 receptor peptide agonist is the quantity that results in a desired therapeutic and/or prophylactic effect without causing unacceptable side effects when administered to a subject in need of VPAC2 receptor stimulation. A “desired therapeutic effect” includes one or more of the following: 1) an amelioration of the symptom(s) associated with the disease or condition; 2) a delay in the onset of symptoms associated with the disease or condition; 3) increased longevity compared with the absence of the treatment; and 4) greater quality of life compared with the absence of the treatment. For example, an “effective amount” of a VPAC2 agonist for the treatment of NIDDM is the quantity that would result in greater control of blood glucose concentration than in the absence of treatment, thereby resulting in a delay in the onset of diabetic complications such as retinopathy, neuropathy, or kidney disease. An “effective amount” of a selective VPAC2 receptor peptide agonist for the prevention of NIDDM is the quantity that would delay, compared with the absence of treatment, the onset of elevated blood glucose levels that require treatment with anti-hypoglycemic drugs such as sulfonylureas, thiazolidinediones, insulin, and/or bisguanidines.

An “effective amount” of the selective VPAC2 receptor peptide agonist administered to a subject will also depend on the type and severity of the disease and on the characteristics of the subject, such as general health, age, sex, body weight and tolerance to drugs. The dose of selective VPAC2 peptide receptor agonist effective to normalize a patient's blood glucose will depend on a number of factors, among which are included, without limitation, the subject's sex, weight and age, the severity of inability to regulate blood glucose, the route of administration and bioavailability, the pharmacokinetic profile of the peptide, the potency, and the formulation.

A typical dose range for the selective VPAC2 receptor peptide agonists of the present invention will range from about 1 μg per day to about 5000 μg per day. Preferably, the dose ranges from about 1 μg per day to about 2500 μg per day, more preferably from about 1 μg per day to about 1000 μg per day. Even more preferably, the dose ranges from about 5 μg per day to about 100 μg per day. A further preferred dose range is from about 10 μg per day to about 50 μg per day. Most preferably, the dose is about 20 μg per day.

A “subject” is a mammal, preferably a human, but can also be an animal, e.g., companion animals (e.g., dogs, cats, and the like), farm animals (e.g., cows, sheep, pigs, horses, and the like) and laboratory animals (e.g., rats, mice, guinea pigs, and the like).

The selective VPAC2 receptor peptide agonists of the present invention can be prepared by using standard methods of solid-phase peptide synthesis techniques. Peptide synthesizers are commercially available from, for example, Rainin-PTI Symphony Peptide Synthesizer (Tucson, Ariz.). Reagents for solid phase synthesis are commercially available, for example, from Glycopep (Chicago, Ill.). Solid phase peptide synthesizers can be used according to manufacturers instructions for blocking interfering groups, protecting the amino acid to be reacted, coupling, decoupling, and capping of unreacted amino acids.

Typically, an α-N-protected amino acid and the N-terminal amino acid on the growing peptide chain on a resin is coupled at room temperature in an inert solvent such as dimethylformamide, N-methylpyrrolidone or methylene chloride in the presence of coupling agents such as dicyclohexylcarbodiimide and 1-hydroxybenzotriazole and a base such as diisopropylethylamine. The α-N-protecting group is removed from the resulting peptide resin using a reagent such as trifluoroacetic acid or piperidine, and the coupling reaction repeated with the next desired N-protected amino acid to be added to the peptide chain. Suitable amine protecting groups are well known in the art and are described, for example, in Green and Wuts, “Protecting Groups in Organic Synthesis”, John Wiley and Sons, 1991, the entire teachings of which are incorporated by reference. Examples include t-butyloxycarbonyl (tBoc) and fluorenylmethoxycarbonyl (Fmoc).

The selective VPAC2 receptor peptide agonists are also synthesized using standard automated solid-phase synthesis protocols using t-butoxycarbonyl- or fluorenylmethoxycarbonyl-alpha-amino acids with appropriate side-chain protection. After completion of synthesis, peptides are cleaved from the solid-phase support with simultaneous side-chain deprotection using standard hydrogen fluoride methods or trifluoroacetic acid (TFA). Crude peptides are then further purified using Reversed-Phase Chromatography on Vydac C18 columns using acetonitrile gradients in 0.1% trifluoroacetic acid (TFA). To remove acetonitrile, peptides are lyophilized from a solution containing 0.1% TFA, acetonitrile and water. Purity can be verified by analytical reversed phase chromatography. Identity of peptides can be verified by mass spectrometry. Peptides can be solubilized in aqueous buffers at neutral pH.

The peptide agonists of the present invention may also be made by recombinant methods known in the art using both eukaryotic and prokaryotic cellular hosts.

Various preferred features and embodiments of the present invention will now be described with reference to the following non-limiting examples.

### EXAMPLE 1

### Preparation of the Selective VPAC2 Receptor Peptide Agonists by Solid Phase t-Boc Chemistry

Approximately 0.5-0.6 grams (0.38-0.45 mmole) Boc Ser(Bzl)-PAM resin is placed in a standard 60 mL reaction vessel. Double couplings are run on an Applied Biosystems ABI430A peptide synthesizer. The following side-chain protected amino acids (2 mmole cartridges of Boc amino acids) are obtained from Midwest Biotech (Fishers, Ind.) and are used in the synthesis:

Arg-Tosyl (TOS), Asp-δ-cyclohexyl ester (CHXL), Glu-δ-cyclohexyl ester (CHXL), His-benzyloxymethyl (BOM), Lys-2-chlorobenzyloxycarbonyl (2Cl-Z), Ser-O-benzyl ether (OBzl), Thr-O-benzyl ether (OBzl), Trp-formyl (CHO) and Tyr-2-bromobenzyloxycarbonyl (2Br-Z) and Boc Gly PAM resin. Trifluoroacetic acid (TFA), di-isopropylethylamine (DIEA), 0.5 M hydroxybenzotriazole (HOBt) in DMF and 0.5 M dicyclohexylcarbodiimide (DCC) in dichloromethane are purchased from PE-Applied Biosystems (Foster City, Calif.). Dimethylformamide (DMF-Burdick and Jackson) and dichloromethane (DCM-Mallinkrodt) is purchased from Mays Chemical Co. (Indianapolis, Ind.).

Standard double couplings are run using either symmetric anhydride or HOBt esters, both formed using DCC. At the completion of the syntheses, the N-terminal Boc group is removed and the peptidyl resins are treated with 20% piperidine in DMF to deformylate the Trp side chain if Trp is present in the sequence. For the N-terminal acylation, four-fold excess of symmetric anhydride of the corresponding acid is added onto the peptide resin. The symmetric anhydride is prepared by diisopropylcarbodiimde (DIC) activation in DCM. The reaction is allowed to proceed for 4 hours and monitored by ninhydrin test. After washing with DCM, the resins are transferred to a TEFLON reaction vessel and are dried in vacuo.

Cleavages are done by attaching the reaction vessels to a HF (hydrofluoric acid) apparatus (Penninsula Laboratories). 1 mL m-cresol per gram/resin is added and 10 mL HF (purchased from AGA, Indianapolis, Ind.) is condensed into the pre-cooled vessel. 1 mL DMS per gram resin is added when methionine is present. The reactions are stirred one hour in an ice bath. The HF is removed in vacuo. The residues are suspended in ethyl ether. The solids are filtered and are washed with ether. Each peptide is extracted into aqueous acetic acid and either is freeze dried or is loaded directly onto a reverse-phase column.

Purifications are run on a 2.2×25 cm VYDAC C18 column in buffer A (0.1% Trifluoroacteic acid in water, B: 0.1% TFA in acetonitrile). A gradient of 20% to 90% B is run on an HPLC (Waters) over 120 minutes at 10 mL/minute while monitoring the UV at 280 nm (4.0 A) and collecting one minute fractions. Appropriate fractions are combined, frozen and lyophilized. Dried products are analyzed by HPLC (0.46×15 cm METASIL AQ C18) and MALDI mass spectrometry.

### EXAMPLE 2

### Preparation of the Selective VPAC2 Receptor Peptide Agonists by Solid Phase FMoc Chemistry

Approximately 114 mg (50 mMole) FMOC Ser(tBu) WANG resin (purchased from GlycoPep, Chicago, Ill.) is placed in each reaction vessel. The synthesis is conducted on a Rainin Symphony Peptide Synthesizer. Analogs with a C-terminal amide are prepared using 75 mg (50 μmole) Rink Amide AM resin (Rapp Polymere. Tuebingen, Germany).

The following FMOC amino acids are purchased from GlycoPep (Chicago, Ill.), and NovaBiochem (La Jolla, Calif.): Arg-2,2,4,6,7-pentamethyldihydrobenzofuran-5-sulfonyl (Pbf), Asn-trityl (Trt), Asp-β-t-Butyl ester (tBu), Glu-δ-t-butyl ester (tBu), Gln-trityl (Trt), His-trityl (Trt), Lys-t-butyloxycarbonyl (Boc), Ser-t-butyl ether (OtBu), Thr-t-butyl ether (OtBu), Trp-t-butyloxycarbonyl (Boc), Tyr-t-butyl ether (OtBu).

Solvents dimethylformamide (DMF-Burdick and Jackson), N-methylpyrrolidone (NMP-Burdick and Jackson), dichloromethane (DCM-Mallinkrodt) are purchased from Mays Chemical Co. (Indianapolis, Ind.).

Hydroxybenzotrizole (HOBt), di-isopropylcarbodiimde (DIC), di-isopropylethylamine (DIEA), and piperidine (Pip) are purchased from Aldrich Chemical Co (Milwaukee, Wis.).

All amino acids are dissolved in 0.3 M in DMF. Three hour DIC/HOBt activated couplings are run after 20 minutes deprotection using 20% Pip/DMF. Each resin is washed with DMF after deprotections and couplings. After the last coupling and deprotection, the peptidyl resins are washed with DCM and are dried in vacuo in the reaction vessel. For the N-terminal acylation, four-fold excess of symmetric anhydride of the corresponding acid is added onto the peptide resin. The symmetric anhydride is prepared by diisopropylcarbodiimde (DIC) activation in DCM. The reaction is allowed to proceed for 4 hours and monitored by ninhydrin test. The peptide resin is then washed with DCM and dried in vacuo.

The cleavage reaction is mixed for 2 hours with a cleavage cocktail consisting of 0.2 mL thioanisole, 0.2 mL methanol, 0.4 mL triisopropylsilane, per 10 mL trifluoroacetic acid (TFA), all purchased from Aldrich Chemical Co., Milwaukee, Wis. If Cys is present in the sequence, 2% of ethanedithiol is added. The TFA filtrates are added to 40 mL ethyl ether. The precipitants are centrifuged 2 minutes at 2000 rpm. The supernatants are decanted. The pellets are resuspended in 40 mL ether, re-centrifuged, re-decanted, dried under nitrogen and then in vacuo.

0.3-0.6 mg of each product is dissolved in 1 mL 0.1% TFA/acetonitrile (ACN), with 20 μL being analyzed on HPLC [0.46×15 cm METASIL AQ C18, 1 mL/min, 45 C.°, 214 nM (0.2 A), A=0.1% TFA, B=0.1% TFA/50% ACN. Gradient=50% B to 90% B over 30 minutes].

Purifications are run on a 2.2×25 cm VYDAC C18 column in buffer A (0.1% trifluoroacteic acid in water, B: 0.1% TFA in acetonitrile). A gradient of 20% to 90% B is run on an HPLC (Waters) over 120 minutes at 10 mL/minute while monitoring the UV at 280 nm (4.0 A) and collecting 1 minute fractions. Appropriate fractions are combined, frozen and lyophilized. Dried products are analyzed by HPLC (0.46×15 cm METASIL AQ C18) and MALDI mass spectrometry.

### EXAMPLE 3

### In Vitro Potency

DiscoveRx: A CHO-S cell line stably expressing human VPAC2 receptor in a 96-well microtiter plate is seeded with 50,000 cells/well the day before the assay. The cells are allowed to attach for 24 hours in 200 μL culture medium. On the day of the experiment, the medium is removed. Also, the cells are washed twice. The cells are incubated in assay buffer plus IBMX for 15 minutes at room temperature. Afterwards, the stimuli are added and are dissolved in assay buffer. The stimuli are present for 30 minutes. Then, the assay buffer is gently removed. The cell lysis reagent of the DiscoveRx cAMP kit is added. Thereafter, the standard protocol for developing the cAMP signal as described by the manufacturer is used (DiscoveRx Inc., USA). EC₅₀ values for cAMP generation are calculated from the raw signal or are based on absolute cAMP levels as determined by a standard curve performed on each plate. In the case of VPAC1 and PAC1 receptors, CHO-PO cells are transiently transfected with human VPAC1 or PAC1 receptor DNA using commercially available transfection reagents (Lipofectamine from Invitrogen). The cells are seeded at a density of 10,000/well in a 96-well plate and are allowed to grow for 3 days in 200 mL culture medium. At day 3, the assay described above for the VPAC2 receptor cell line is performed.

Results for each agonist are the mean of two independent runs. VPAC1 and PAC1 results are only generated using the DiscoveRx assay. The typically tested concentrations of peptide are: 1000, 300, 100, 10, 1, 0.3, 0.1, 0.01, 0.001, 0.0001 and 0 nM.

Alpha screen: Cells are washed in the culture flask once with PBS. Then, the cells are rinsed with enzyme free dissociation buffer. The dissociated cells are removed. The cells are then spun down and washed in stimulation buffer. For each data point, 50,000 cells suspended in stimulation buffer are used. To this buffer, Alpha screen acceptor beads are added along with the stimuli. This mixture is incubated for 60 minutes. Lysis buffer and Alpha screen donor beads are added and are incubated for 60 to 120 minutes. The Alpha screen signal (indicative of intracellular cAMP levels) is read in a suitable instrument (e.g. AlphaQuest from Perkin-Elmer). Steps including Alpha screen donor and acceptor beads are performed in reduced light. The EC₅₀ for cAMP generation is calculated from the raw signal or is based on absolute cAMP levels as determined by a standard curve performed on each plate.

Results for each agonist are, at minimum, from two analyses performed in a single run. For some agonists, the results are the mean of more than one run. The tested peptide concentrations are: 10000, 1000, 100, 10, 3, 1, 0.1, 0.01, 0.003, 0.001, 0.0001 and 0.00001 nM. The activity (EC₅₀ (nM)) for the human VPAC2, VPAC1, and PAC1 receptors is reported in Table 1

TABLE 1

| | | Human | | |
|---|---|---|---|---|
| | Human | VPAC2 | Human | Human |
| | VPAC2 | Receptor: | VPAC1 | PAC1 |
| | Receptor: | Alpha | Receptor: | Receptor: |
| Agonist # | DiscoveRx¹ | Screen² | DiscoveRx¹ | DiscoveRx¹ |
| PACAP-27 | 0.84 | 2.33 | 0.05 | 0.06 |
| VIP (SEQ ID | 0.70 | 1.00 | 0.02 | 15.4 |
| NO: 1) | | | | |
| VPAC1-P1 | 179.29 | | | |
| P100 | | 0.28 | | |
| P101 | | 0.21 | | |
| P102 | | 27.38 | | |
| P103 | | 0.12 | | |
| P104 | | 0.27 | | |
| P105 | | 8.33 | | |
| P106 | | 0.16 | | |
| P107 | | 0.12 | | |
| P108 | | 0.27 | | |
| P109 | | 0.22 | | |
| P110 | | 0.13 | | |
| P111 | | 0.34 | | |
| P112 | | 0.16 | | |
| P113 | | 0.26 | | |
| P114 | | 0.14 | | |
| P115 | | 0.20 | | |
| P116 | | 0.12 | | |
| P117 | | 0.20 | | |
| P118 | | 0.26 | | |
| P119 | 0.34 | 0.42 | | |
| P120 | | 0.06 | | |
| P121 | | 0.10 | | |
| P122 | | 0.14 | | |
| P123 | | 0.12 | | |
| P124 | | 0.09 | | |
| P125 | | 0.14 | | |
| P127 | | 0.22 | | |
| P129 | | 0.49 | | |
| P130 | | 0.77 | | |
| P131 | | 22.28 | | |
| P132 | | 3.10 | | |
| P133 | | 2.70 | | |
| P134 | | 5.47 | | |
| P135 | | 0.18 | | |
| P138 | | 0.45 | | |
| P139 | | 0.20 | | |
| P14 | 379.01 | | | |
| P140 | | 0.07 | | |
| P141 | | 0.72 | | |
| P142 | | 0.15 | | |
| P143 | | 0.10 | | |
| P144 | | 0.32 | | |
| P146 | | 0.17 | | |
| P147 | | 0.13 | | |
| P148 | | 0.10 | | |
| P149 | | 0.41 | | |
| P150 | | 0.18 | | |
| P151 | | 0.07 | | |
| P152 | | 0.17 | | |
| P153 | | 0.11 | | |
| P154 | | 0.18 | | |
| P155 | | 0.24 | | |
| P158 | | 0.11 | | |
| P159 | | 0.17 | | |
| P160 | | 13.16 | | |
| P161 | | 4.00 | | |
| P162 | | 11.07 | | |
| P163 | | 5.53 | | |
| P164 | | 0.11 | | |
| P165 | | 0.94 | | |
| P166 | | 0.15 | | |
| P167 | | 0.17 | | |
| P168 | | 0.27 | | |
| P169 | | 0.14 | | |
| P170 | | 0.18 | | |
| P171 | | 0.82 | | |
| P172 | | 0.29 | | |
| P173 | | 0.25 | | |
| P174 | | 0.26 | | |
| P175 | | 0.68 | | |
| P176 | | 0.28 | | |
| P177 | | 0.21 | | |
| P179 | | 0.24 | | |
| P18 | 2.88 | 3.31³ | 0.31 | 435.0 |
| P18* | 2.87 | 3.31 | 0.4 | >50.0 |
| P180 | | 1.04 | | |
| P181 | | 0.13 | | |
| P182 | | 0.29 | | |
| P183 | | 0.14 | | |
| P184 | | 0.22 | | |
| P185 | | 0.18 | | |
| P186 | | 0.15 | | |
| P187 | | 0.40 | | |
| P188 | | 0.32 | | |
| P189 | | 0.31 | | |
| P19 | 9.03 | | 2.9 | |
| P192 | | 0.18 | | |
| P193 | | 0.18 | | |
| P194 | | 0.13 | | |
| P195 | | 0.25 | | |
| P196 | | 0.23 | | |
| P197 | | 0.09 | | |
| P199 | | 29.65 | | |
| P20 | 2.13 | 2.77 | 2.0 | >25 |
| P201 | | 7.82 | | |
| P203 | | 0.14 | | |
| P204 | | 2418.74 | | |
| P205 | | 1.14 | | |
| P207 | | 0.86 | | |
| P208 | | 50.15 | | |
| P209 | | 0.10 | | |
| P21 | 6.87 | 11.60 | 10.70 | >100 |
| P210 | | 0.91 | | |
| P211 | | 988.66 | | |
| P212 | | 0.12 | | |
| P213 | | 0.24 | | |
| P214 | | 0.16 | | |
| P215 | | 0.09 | | |
| P216 | | 0.11 | | |
| P22 | 1.18 | 2.23 | 0.9 | 73.5 |
| P220 | | 0.12 | | |
| P221 | | 1.98 | | |
| P222 | | 0.51 | | |
| P223 | | 5.82 | | |
| P224 | | 0.14 | | |
| P228 | | 3.48 | | |
| P229 | | 2.60 | | |
| P23 | 1.41 | 2.30 | 0.9 | 98.3 |
| P230 | | 0.30 | | |
| P231 | | 0.26 | | |
| P232 | | 1.94 | | |
| P233 | | 0.11 | | |
| P234 | | 0.16 | | |
| P235 | | 0.82 | | |
| P236 | | 0.12 | | |
| P24 | 5.78 | | 2.5 | >100 |
| P240 | | 0.51 | | |
| P241 | | 2.17 | | |
| P242 | | 0.80 | | |
| P243 | | 0.28 | | |
| P244 | | 2.03 | | |
| P249 | | 0.93 | | |
| P25 | 3.02 | | 2.7 | 136.8 |
| P250 | | 0.18 | | |
| P251 | | 0.92 | | |
| P252 | | 0.26 | | |
| P253 | | 0.46 | | |
| P258 | | 0.17 | | |
| P259 | | 1.08 | | |
| P26 | 4.81 | | 0.9 | 84.0 |
| P260 | | 0.83 | | |
| P261 | | 0.09 | | |
| P262 | | 0.24 | | |
| P263 | | 0.07 | | |
| P264 | | 0.10 | | |
| P265 | | 0.21 | | |
| P269 | | 1.02 | | |
| P27 | 27.65 | | 5.2 | >100 |
| P270 | | 0.13 | | |
| P271 | | 0.12 | | |
| P275 | | 0.32 | | |
| P279 | | 5.37 | | |
| P28 | 3.92 | | 2.6 | 53.6 |
| P284 | | 0.18 | | |
| P289 | | 0.16 | | |
| P29 | 3.02 | | 18.3 | |
| P290 | | 0.22 | | |
| P291 | | 0.36 | | |
| P292 | | 0.23 | | |
| P293 | | 1.89 | | |
| P294 | | 0.24 | | |
| P295 | | 0.39 | | |
| P296 | | 6.69 | | |
| P297 | | 0.36 | | |
| P298 | | 0.50 | | |
| P301 | | 0.89 | | |
| P302 | | 0.45 | | |
| P305 | | 0.17 | | |
| P307 | | 0.32 | | |
| P308 | | 0.26 | | |
| P31 | 0.11 | 0.13 | 6.0 | 115.8 |
| P314 | | 0.22 | | |
| P315 | | 0.19 | | |
| P316 | | 0.30 | | |
| P317 | | 0.36 | | |
| P318 | | 0.32 | | |
| P319 | | 0.26 | | |
| P320 | | 0.49 | | |
| P321 | | 0.38 | | |
| P322 | | 0.22 | | |
| P323 | | 0.23 | | |
| P324 | | 0.23 | | |
| P325 | | 0.24 | | |
| P326 | | 0.21 | | |
| P329 | | 0.31 | | |
| P33 | 0.86 | 0.73 | 4.7 | 111.5 |
| P333 | | 0.75 | | |
| P335 | | 0.22 | | |
| P338 | | 0.46 | | |
| P34 | 2.28 | 2.44 | 0.8 | 80.5 |
| P341 | | 0.38 | | |
| P342 | | 0.21 | | |
| P344 | | 0.13 | | |
| P345 | | 1.17 | | |
| P346 | | 0.23 | | |
| P349 | | 0.08 | | |
| P35 | 182.07 | | 13.7 | 1000.0 |
| P350 | | 0.13 | | |
| P351 | | 0.11 | | |
| P352 | | 0.14 | | |
| P353 | | 0.18 | | |
| P354 | | 0.14 | | |
| P355 | | 0.10 | | |
| P358 | | 0.10 | | |
| P359 | | 0.09 | | |
| P364 | | 0.11 | | |
| P365 | | 0.13 | | |
| P366 | | 2.60 | | |
| P37 | 1.68 | | 6.4 | 35.6 |
| P42 | 126.42 | | 1.4 | |
| P43 | 5.37 | | 75.6 | 104.0 |
| P44 | 0.92 | | 5.5 | 36.6 |
| P45 | 11.64 | | 1.75 | 315.5 |
| P46 | 121.01 | | 57.5 | >1000 |
| P47 | 1.07 | 1.39 | 3.6 | 136.2 |
| P48 | 9.55 | | 9.93 | 95.9 |
| P49 | 13.41 | | 22.8 | 44.2 |
| P50 | 19.25 | | 110.6 | |
| P51 | 91.09 | | 3.61 | >1000 |
| P52 | 140.05 | | 37.52 | >1000 |
| P53 | 50.47 | | 117.9 | >1000 |
| P54 | 32.78 | | 84.1 | >1000 |
| P55 | 15.32 | | 133.5 | >1000 |
| P56 | 108.54 | | 9.96 | 404.7 |
| P58 | 11.79 | | 44.1 | 35.6 |
| P59 | 1000.00 | | 1000.0 | |
| P6 | 2.56 | 3.19 | 5.0 | >40 |
| P61 | 2.14 | | 2.58 | 11.6 |
| P62 | 13.23 | | 17 | 137.2 |
| P63 | >1000 | | >1000 | 0.9 |
| P64 | >1000 | | 221.3 | 0.1 |
| P65 | >1000 | | >1000 | 0.7 |
| P66 | >1000 | | <1000 | 1.4 |
| P67 | | 94.14 | | |
| P68 | 20.33 | | 41 | 94.7 |
| P69 | 6.39 | | 8.48 | 103.6 |
| P7 | 2.78 | | | |
| P70 | 4.89 | | 27.4 | 20.7 |
| P71 | 3.14 | | 0.2 | 14.6 |
| P72 | 1.79 | | 0.3 | 35.3 |
| P74 | 2.72 | | 0.4 | 145.3 |
| P75 | 3.44 | | 0.65 | 13.4 |
| P76 | 3.88 | | 0.8 | 15.9 |
| P8 | 1.61 | | | |
| P82 | | 15.24 | | |
| P84 | | 185 | | |
| P85 | | 22.2 | | |
| P87 | | >10000 | | |
| P88 | >1000 | 1718.93 | | |
| P89 | 0.7 | 1.23 | | |
| P9 | 2.41 | | | |
| P92 | 8.2 | 10.18 | | |
| P94 | >1000 | 696 | | |
| P98 | | 0.15 | | |
| P99 | | 0.14 | | |

¹EC₅₀ (nM); Mean of two independent runs
²EC₅₀ (nM); Single result from two analyses performed in a single run
³Mean of two separate results for the given assay
⁴NA = Not assayed (for all NA entries)

### EXAMPLE 4

### Selectivity

Binding assays: Membrane prepared from a stable VPAC2 cell line (see Example 3) or from cells transiently transfected with human VPAC1 or PAC1 are used. A filter binding assay is performed using 125I-labeled VIP for VPAC1 and VPAC2 and 125I-labeled PACAP-27 for PAC1 as the tracers.

For this assay, the solutions and equipment include:

Presoak solution: 0.5% Polyethyleneamine in Aqua dest.

Buffer for flushing filter plates: 25 mM HEPES pH 7.4

Blocking buffer: 25 mM HEPES pH 7.4; 0.2% protease free BSA

Assay buffer: 25 mM HEPES pH 7.4; 0.5% protease free BSA

Dilution and assay plate: PS-Microplate, U form

Filtration Plate Multiscreen FB Opaque Plate; 1.0 μM Type B Glasfiber filter

In order to prepare the filter plates, aspirate the presoak solution by vacuum filtration. Flush the plates twice with 200 μL flush buffer. Add 200 μL blocking buffer to the filter plate. The filter plate is then incubated with 200 μL presoak solution for 1 hour at room temperature.

Fill the assay plate with 25 μL assay buffer, 25 μL membranes (2.5 μg) suspended in assay buffer, 25 μL compound (agonist) in assay buffer, and 25 μL tracer (about 40000 cpm) in assay buffer. Incubate the filled plate for 1 hour with shaking.

Conduct the transfer from assay plate to filter plate. Aspirate the blocking buffer by vacuum filtration and wash two times with flush buffer. Transfer 90 μL from the assay plate to the filter plate. Aspirate the 90 μL transferred from assay plate and wash three times with 200 μL flush buffer. Remove the plastic support. Dry for 1 hour at 60° C. Add 30 μL Microscint. Perform the count.

The selectivity (IC₅₀) for human VPAC2, VPAC1, and PAC1 is reported in Table 2.

TABLE 2

| | Human VPAC2 | Human VPAC1 | Human PAC1 |
|---|---|---|---|
| Agonist # | Receptor Binding | Receptor Binding | Receptor Binding |
| PACAP-27 | 2.76 | 3.63 | 9.1 |
| VIP (SEQ ID | 5.06 | 3.28 | >25000 |
| NO: 1) | | | |
| P100 | 0.40 | 321.05 | 186.7 |
| P101 | 0.18 | 93.57 | 274.3 |
| P102 | 15.42 | 183.51 | 55.0 |
| P103 | 0.08 | 228.28 | 333.1 |
| P104 | 0.68 | 301.08 | 117.2 |
| P105 | 5.41 | 263.0 | 127.1 |
| P106 | 0.22 | 117.79 | 1817.2 |
| P107 | 0.16 | 121.9 | 225.2 |
| P108 | 0.45 | 331.9 | 485.8 |
| P109 | 0.11 | 274.3 | 257.3 |
| P110 | 0.10 | 424.6 | 320.9 |
| P111 | 0.19 | 300.0 | 258.3 |
| P112 | 0.49 | 192.65 | 164.0 |
| P113 | 0.69 | 293.29 | 189.3³ |
| P114 | 0.56 | 170.85 | 188.1³ |
| P115 | 0.10 | 266.82 | 189.6 |
| P116 | 0.74 | 369.05 | 145.3 |
| P117 | 0.51 | 442.36 | 243.2 |
| P119 | 0.48 | >3000 | >25000 |
| P120 | 0.44 | | 577.0 |
| P121 | 0.19 | 169.3 | 252.8³ |
| P122 | 0.17 | 77.6 | 303.9³ |
| P123 | 0.26 | 166.0 | 265.4³ |
| P124 | 0.2 | 211.2 | 264.2³ |
| P125 | 0.19 | 178.9 | 299.7³ |
| P127 | 0.23 | 289.84 | 209.2 |
| P129 | 0.38 | 461.64 | 678.2 |
| P130 | 0.43 | 232.66 | 444.3 |
| P131 | 5.67 | 1372.89 | 631.5 |
| P132 | 1.51 | 616.90 | 1022.7 |
| P133 | 5.30 | 469.91 | 1938.0 |
| P134 | 2.13 | 832.58 | 479.1 |
| P135 | 0.43 | 251.24 | 747.8 |
| P138 | 1.01 | 5.28 | 1113.2 |
| P139 | 0.34 | 234.71 | >25000 |
| P140 | 1.94 | 1136.71 | 485.3 |
| P141 | 0.64 | 25.91 | 658.0 |
| P142 | 0.58 | 363.55 | 1191.4 |
| P143 | 0.50 | 605.08 | 1082.0 |
| P144 | 0.57 | 4.43 | 1183.7 |
| P146 | 0.52 | 400.85 | 1540.8 |
| P147 | 0.24 | 106.79 | 464.0 |
| P148 | 0.77 | 247.37 | 1003.5 |
| P149 | 0.61 | 212.32 | 773.5 |
| P150 | 0.25 | 444.11 | 916.2 |
| P151 | 0.72 | 203.65 | 720.4 |
| P152 | 0.37 | 514.09 | 671.6 |
| P153 | 0.70 | 180.58 | 1127.1 |
| P154 | 0.31 | 140.90 | 1960.1 |
| P155 | 0.71 | 231.22 | 148.5 |
| P158 | 0.21 | 25.71 | |
| P159 | 0.17 | 131.23 | |
| P160 | 4.77 | 50.31 | 262.0 |
| P161 | 6.61 | 118.51 | 4259.0 |
| P162 | 3.72 | 5.14 | 186.1 |
| P163 | 2.00 | 35.68 | 237.9 |
| P164 | 2.46 | >3000 | 9368.8 |
| P165 | 18.38 | 486.02 | 2862.2 |
| P166 | 0.46 | 248.03 | 126.8 |
| P167 | 1.14 | 1153.0 | 1010.0 |
| P168 | 0.21 | 233.64 | 537.7 |
| P169 | 0.26 | 240.0 | 482.0 |
| P170 | 0.34 | 275.20 | 199.8 |
| P171 | 5.81 | 174.32 | 335.1 |
| P172 | 3.58 | 411.67 | 500.3 |
| P173 | 0.55 | 1787.03 | 755.6 |
| P174 | 55.07 | 302.47 | 620.8 |
| P175 | 13.31 | >3000 | >25000 |
| P176 | 6.88 | 93.05 | 226.8 |
| P177 | 0.70 | 127.68 | 276.0 |
| P179 | 0.42 | 390.05 | |
| P180 | 5.63 | >3000 | 589.3 |
| P181 | 0.29 | 325.19 | 521.5 |
| P182 | 0.17 | 205.03 | 428.8 |
| P183 | 0.26 | 423.64 | 505.5 |
| P184 | 0.23 | 324.43 | 338.9 |
| P185 | 0.41 | 358.01 | |
| P186 | 0.87 | 835.09 | 318.2 |
| P187 | 0.17 | 134.34 | 295.8 |
| P188 | 0.29 | 6.19 | 700.4 |
| P189 | 0.20 | 6.87 | 303.4 |
| P191 | 60.61 | 1277.23 | >25000 |
| P192 | 0.53 | 1432.29 | 14311.8 |
| P193 | 0.37 | 249.93 | 541.7 |
| P194 | 0.28 | 172.86 | 625.5 |
| P195 | 2.34 | >3000 | 139.3 |
| P196 | 0.85 | 453.46 | >25000 |
| P197 | 0.31 | 446.72 | |
| P199 | 8.26 | 429.11 | 244.3 |
| P20 | 2.50³ | 98.7³ | |
| P203 | 0.18 | | |
| P207 | 0.81 | 282.27 | |
| P209 | 0.20 | 130.14 | 342.7 |
| P21 | 4.94³ | 384.7³ | |
| P210 | 0.68 | 395.02 | |
| P212 | 0.19 | 197.91 | |
| P213 | 0.27 | 32.25 | |
| P214 | 0.19 | 14.51 | |
| P22 | 0.87³ | 123.0³ | |
| P220 | 0.25 | 5.86 | 848.8 |
| P222 | 1.05 | 176.98 | 465.7 |
| P224 | 0.35 | 207.86 | 209.7 |
| P23 | 0.92³ | 149.9³ | |
| P230 | 0.75 | 660.65 | 462.6 |
| P231 | 0.55 | 457.81 | 300.0 |
| P233 | 0.31 | 323.46 | 319.9 |
| P234 | 0.33 | 640.85 | 248.3 |
| P235 | 1.47 | 1335.57 | 1113.1 |
| P236 | 0.37 | 588.91 | 225.2 |
| P24 | 1.97³ | 198.4³ | |
| P240 | 6.08 | >3000 | >25000 |
| P241 | 18.09 | >3000 | >25000 |
| P242 | 6.78 | >3000 | >25000 |
| P243 | 5.51 | >3000 | >25000 |
| P244 | 26.86 | >3000 | >25000 |
| P249 | 6.59 | >3000 | >25000 |
| P25 | 1.65³ | 233.4³ | |
| P250 | 0.63 | 854.93 | >25000 |
| P251 | 2.33 | >3000 | >25000 |
| P252 | 0.98 | 1180.24 | >25000 |
| P253 | 1.78 | 2162.63 | >25000 |
| P258 | 0.63 | >3000 | >25000 |
| P259 | 13.21 | 3462.0 | >3000 |
| P260 | 4.79 | 3318.0 | >3000 |
| P261 | 0.2 | 1004.0 | >25000 |
| P262 | 0.61 | >3000 | >25000 |
| P263 | 0.38 | 2682.0 | 636 |
| P264 | 0.88 | >3000 | >25000 |
| P265 | 0.68 | >3000 | >25000 |
| P269 | 10.09 | >3000 | >25000 |
| P270 | 0.15 | 110.06 | 436.2 |
| P271 | 0.14 | 67.57 | 352.3 |
| P275 | 0.38 | 557.68 | 629.8 |
| P284 | 0.38 | >3000 | >25000 |
| P289 | 0.38 | 259.58 | 323.9 |
| P290 | 0.27 | 220.62 | 209.1 |
| P291 | 0.45 | >3000 | >25000 |
| P292 | 0.3 | >3000 | >25000 |
| P293 | 16.71 | >3000 | >25000 |
| P294 | 0.45 | >3000 | >25000 |
| P295 | 0.75 | >3000 | >25000 |
| P296 | >100 | >3000 | >25000 |
| P297 | 1.95 | >3000 | >25000 |
| P298 | 8.20 | >3000 | >25000 |
| P301 | 7.27 | >3000 | >25000 |
| P302 | 1.62 | >3000 | >25000 |
| P305 | 0.17 | 268.13 | 171.2 |
| P307 | 0.28 | 274.09 | 293.4 |
| P308 | 0.34 | 261.59 | |
| P31 | 0.23 | 145.13 | 405.7 |
| P314 | 0.57 | >3000 | >25000 |
| P315 | 0.29 | >3000 | |
| P316 | 0.38 | >3000 | |
| P317 | 1.23 | >3000 | >25000 |
| P318 | 0.63 | >3000 | >25000 |
| P319 | 0.69 | >3000 | >25000 |
| P320 | 1.20 | >3000 | |
| P321 | 0.29 | 429.44 | |
| P322 | 0.40 | >3000 | >25000 |
| P323 | 0.32 | >3000 | |
| P324 | 0.43 | >3000 | |
| P325 | 0.40 | >3000 | >25000 |
| P326 | 0.33 | >3000 | >25000 |
| P329 | 1.41 | >3000 | >25000 |
| P33 | 2.31³ | 120.0³ | |
| P333 | 9.71 | >3000 | |
| P335 | 0.39 | >3000 | |
| P338 | 0.76 | >3000 | |
| P34 | 0.92³ | 79.2³ | |
| P341 | 2.39 | >3000 | |
| P342 | 0.35 | >3000 | |
| P344 | 0.25 | >3000 | |
| P345 | 19.26 | >3000 | |
| P346 | 0.25 | >3000 | |
| P352 | 0.60 | >3000 | |
| P47 | 0.84 | 123.71 | |
| P6 | 2.58³ | 194.8³ | 334.4 |
| P71 | 1.52 | 73.07 | |
| P72 | 1.52 | 158.95 | |
| P82 | 1.9 | 195.0 | |
| P84 | 20 | 469.0 | |
| P85 | 1.7 | 226.0 | |
| P87 | 1106 | 636.0 | |
| P88 | 93.9 | 979.0 | |
| P89 | 0.15 | 162.0 | |
| P92 | 0.86 | 74.6 | |
| P94 | 71.5 | 271.0 | |
| P98 | 0.17 | 69.0 | 96.3 |
| P99 | 1.72 | 394.45 | 2056.7 |

¹No affinity for the human PAC1 receptor
²NA = Not assayed (for all NA entries)
³Mean of separate results for the given assay

Comparison of the Interaction of VIP, P31, P104, and P119 with the Recombinant Rat VPAC1, VPAC2 and PAC1 Receptors Expressed in CHO Cells:

The peptide samples are stored frozen and thawed prior to the assay. Reference compounds (Eg. VIP and the tracers) are not stored frozen. All peptide sample and reference compound dilutions are performed in PBS. Peptides solutions are kept in the cold room for four days. Stock solutions are stored at −80° C. New dilution curves are prepared every week.

All studies are performed on crude membranes prepared from three different cell cultures expressing the different recombinant receptors, using methodology that is known in the literature. Duplicate values are obtained for each assay.

The selectivity of the VPAC2 receptor peptide agonists of the present invention are tested on the rat VPAC1 and VPAC2 receptors recombinantly expressed in CHO cells. The compound of the invention were evaluated in receptor binding & adenylate cyclase activation assays.

Competion binding curves from 10⁻¹¹ to 10⁻⁵ M (two concentrations per log) of unlabelled peptide using ¹²⁵I-VIP (VPAC1-R) and ¹²⁵I-RO 25-1553 (VPAC2-R) as tracers; incubations performed at 25° C. for 30 minutes. In each series of assays, unlabelled VIP and RO 25-1553 are used as standards. Each assay is done in duplicate and performed on two different membrane preparations.

TABLE 3
Binding data are expressed as the IC₅₀ values of tracer inhibition.
A different reference compound is used as the tracer for the VPAC1
receptor and for the VPAC2 receptor. The last column represents the
ratio between the IC₅₀ values on VPAC1 and VPAC2 receptor
and is, thus, an index of selectivity for a receptor subtype. VIP has a three-
fold preference for the VPAC1 receptor whereas all the other molecules
tested indicated a clear preference for the VPAC2 receptor. The values
are given with the standard error.

| | RAT VPAC1 | RAT VPAC2 | | |
|---|---|---|---|---|
| Peptide | IC50 (log M) | IC50 (log M) | VPAC1/VPAC2 | N |
| VIP | 8.526 ± 0.190 | 8.108 ± 0.200 | 0.38 | 3 |
| P 31 | 6.678 ± 0.089 | 9.415 ± 0.090 | 546 | 3 |
| P 104 | 5.754 ± 0.236 | 8.744 ± 0.101 | 977 | 3 |
| P 119 | 7.534 ± 0.115 | 9.209 ± 0.040 | 47 | 3 |
| VIP, 2^{nd} run | 9.26 ± 0.40 | 8.54 ± 0.062 | 0.19 | 2 |
| P 261 | 6.89 ± 0.04 | 9.45 ± 0.16 | 363 | 3 |
| P 292 | 6.69 ± 0.22 | 9.12 ± 0.04 | 269 | 3 |

II Adenylate Cyclase Activation)

Dose-effect curves of adenylate cyclase activation were generated using the VPAC2 receptor peptide agonists (10⁻¹¹ to 10⁻⁶ M, two concentrations per log) of the present invention.

Adenylate cyclase activity was determined by the procedure of Salomon et al. (1974), A highly sensitive adenylate cyclase assay. Analytical Biochemistry 58 (1974. Membrane proteins (3-15 g) are incubated in a total volume of 60 l containing 0.5 mM [³²P]-ATP, 10 M GTP, 5 mM MgCl₂, 0.5 mM EGTA, 1 mM cAMP, 1 mM theophylline, 10 mM phospho(enol)pyruvate, 30 g/ml pyruvate kinase and 30 mM Tris-HCl at a final pH of 7.8. The reaction is initiated by membrane addition and is terminated after 15 min incubation at 37° C. by addition of 0.5 ml of 0.5% sodium dodecyl-sulfate solution containing 0.5 mM ATP, 0.5 mM cAMP and 20,000 g [³H]-cAMP. cAMP was separated from ATP by two successive chromatographies on Dowex 50Wx8 and neutral alumina.

TABLE 4
Functional data are obtained by measuring adenylate cyclase activation on
membrane preparations expressing the rat VPAC1 and VPAC2 receptors. The values are
provided as the EC₅₀ result (the ratio between the EC₅₀ and the maximal increase in cyclic
AMP production (over basal) per minute and per mg protein). The values are given with the
standard error.

| | VPAC1 | VPAC2 | | VPAC1 | VPAC2 |
|---|---|---|---|---|---|
| Peptide | EC₅₀ (log M) | EC₅₀ (log M) | VPAC1/VPAC2 | cAMP max | cAMP max |
| VIP | 8.831 ± 0.140 | 8.102 ± 0.080 | 0.19 | 224 ± 18 | 152 ± 15 |
| P 31 | 7.599 ± 0.091 | 9.840 ± 0.083 | 174 | 200 ± 13 | 134 ± 12 |
| P 104 | 6.731 ± 0.187 | 8.996 ± 0.098 | 184 | 195 ± 18 | 129 ± 19 |
| P 119 | 8.538 ± 0.317 | 10.040 ± 0.229 | 32 | 207 ± 13 | 133 ± 18 |
| VIP, 2^{nd} run | 9.23 ± 0.252 | 8.456 ± 0.178 | 0.17 | 135 | 80.7 |
| P 261 | 7.90 ± 0.43 | 10.20 ± 0.35 | 200 | 126 | 78.5 |
| P 292 | 7.94 ± 0.39 | 9.94 ± 0.48 | 100 | 124 | 79.1 |

VIP, PACAP, P31, P104 and P119 were also evaluated in binding and adenylate cyclase assays. For VIP, P31, P104 and P119 binding could not be reliably determined due to incomplete tracer displacement at the maximal concentration tested (10 μM peptide). In the adenylate cyclase assay the potency ratio compared to PACAP-27 was >200 for VIP, P31 and P119 and >1000 for P104.

TABLE 5
In vitro potency using DiscoveRx (See Example 3). CHO-PO cells
are transiently transfected with rat VPAC 1 or VPAC 2 receptor DNA.
The activity (EC₅₀ (nm)) for these receptors is reported in the
table below.

| | Rat VPAC 2 | Rat VPAC 1 |
|---|---|---|
| Agonist # | Receptor DiscoveRx | Receptor DiscoveRx |
| P31 | | 0.44 |
| P89 | | 0.02 |
| PACAP-27 | | 0.07 |
| VIP | 0.50 | 0.02 |
| 2121156 | | 0.34 |
| P104 | 0.51 | 6.75 |
| P115 | | 0.95 |
| P118 | | 0.47 |
| P119 | | 0.07 |
| P120 | | 0.42 |
| P121 | | 0.66 |
| P135 | 0.05 | 0.02 |
| P140 | | 5.81 |
| P143 | | 3.81 |
| P150 | 0.10 | |
| P154 | | 0.85 |
| P167 | 0.24 | 6.88 |
| P168 | 0.05 | |
| P169 | 0.07 | 1.32 |
| P170 | 0.15 | |
| P172 | 0.11 | |
| P177 | 0.10 | |
| P222 | 34.92 | 43.89 |
| P240 | 0.36 | 24.54 |
| P241 | | 61.06 |
| P242 | 0.46 | 72.21 |
| P243 | 0.79 | 156.85 |
| P244 | 1.85 | 127.40 |
| P251 | | 212.12 |
| P261 | 0.04 | 0.35 |
| P264 | 0.10 | 4.01 |
| P270 | | 0.67 |
| P284 | 0.07 | 0.44 |
| P291 | 0.06 | 1.12 |
| P292 | 0.07 | 1.91 |
| P305 | 0.04 | 3.71 |
| P308 | 0.09 | 9.74 |
| P314 | 0.12 | 29.25 |
| P315 | 0.04 | 1.18 |
| P318 | 0.09 | 9.96 |
| P321 | 0.04 | 0.11 |
| P322 | 0.04 | 0.50 |
| P323 | 0.07 | 4.41 |
| P325 | 0.04 | 1.46 |
| P326 | 0.09 | 4.53 |
| P329 | 0.15 | 4.87 |
| P333 | 0.13 | 1.37 |
| P335 | 0.05 | 0.94 |
| P338 | 0.07 | 5.16 |
| P342 | 0.06 | 2.39 |
| P344 | 0.06 | 1.56 |
| P346 | 0.06 | 3.02 |
| P352 | 0.09 | 4.35 |
| P356 | NA | NA |

### EXAMPLE 5

### In Vivo Assays

Intravenous glucose tolerance test (IVGTT): Normal Wistar rats are fasted overnight and are anesthetized prior to the experiment. A blood sampling catheter is inserted into the rats. The compound is given in the jugular vein. Blood samples are taken from the carotid artery. A blood sample is drawn immediately prior to the injection of glucose along with the compound. After the initial blood sample, glucose mixed with compound is injected intravenously (i.v.). A glucose challenge of 0.5 g/kg body weight is given, injecting a total of 1.5 mL vehicle with glucose and agonist per kg body weight. The peptide concentration vary to produce the desired dose in μg/kg. Blood samples are drawn at 2, 4, 6 and 10 minutes after giving glucose. The control group of animals receives the same vehicle along with glucose, but with no compound added. In some instances, a 30 minute post-glucose blood sample is drawn. Aprotinin is added to the blood sample (250 kIU/ml blood). The serum is then analyzed for glucose and insulin using standard methodologies.

The assay uses a formulated and calibrated peptide stock in PBS. Normally, this stock is a prediluted 100 μM stock. However, a more concentrated stock with approximately 1 mg agonist per mL is used. The specific concentration is always known. Variability in the maximal response is mostly due to variability in the vehicle dose.

Protocol details are as follows:

| SPECIES/STRAIN/WEIGHT | Rat/Wistar Unilever/approximately 275-300 g |
|---|---|
| TREATMENT DURATION | Single dose |
| DOSE VOLUME/ROUTE | 1.5 mL/kg/iv |
| VEHICLE | 8% PEG300, 0.1% BSA in water |
| FOOD/WATER REGIMEN | Rats are fasted overnight prior to surgery. |
| LIVE-PHASE PARAMETERS | Animals are sacrificed at the end of the test. |
| IVGTT: Performed on rats (with two | Glucose IV bolus: 500 mg/kg as 10% |
| catheters, jugular vein and carotid | solution (5 mL/kg) at time = 0. |
| artery) of each group, under | Compound iv: Just after glucose. |
| pentobarbital anesthesia. | Blood samplings (300 μL from carotid artery; |
| | EDTA as anticoagulant; aprotinin and PMSF |
| | as antiproteolytics; kept on ice): 0, 2, 4, 6, and |
| | 10 minutes. |
| | Parameter determined: Insulin. |
| TOXICOKINETICS | Plasma samples remaining after insulin |
| | measurements are kept at −20° C. and are sent |
| | to Hamburg for determination of compound |
| | levels. |
| NUMBER OF SAMPLES | 150 |

TABLE 6

| | % increase | % increase | % increase | IVGTT |
|---|---|---|---|---|
| | AUC: Dose = | AUC: Dose = | AUC: Dose = | (ED50; |
| Peptide | 0.1 μg/kg | 0.5 μg/kg | 10 μg/kg | μg/kg) |
| P31 | NA | 250 | NA | NA |
| P44 | 19 | NA | 194 | NA |
| P89 | NA | 110 | 280 | NA |
| P104 | NA | 142, 205² | 198, 370² | 0.09 |
| P119 | NA | 118 | 240 | 0.3 |
| P261 | NA | 136 | 320 | NA |
| P264 | NA | 10 | 95 | NA |
| P292 | NA | 292 | 370 | NA |

¹NA = Not assayed (for all NA entries)
²Analysis from two independent experiments.
AUC = Area under curve

Delayed IVGTT: Perform IVGTT as described above, making the following changes. After the initial blood sample, compound or vehicle is injected i.v. Glucose is injected i.v. 30 minutes later in a separate injection. Blood samples are taken immediately prior to administration of the compound, at 15 minutes after administration of the compound, and at 30 minutes after administration of the compound. The sample at 30 minutes after administration of the compound is taken immediately prior to glucose administration. Blood samples are drawn 2, 4, 6, 10, and 30 minutes after giving glucose (i.e. 32, 34, 36, 40 and 60 minutes after compound administration). The blood samples at 15 and 60 minutes are not essential to the study and not always taken. Aprotinin is added to the blood sample (250 kIU/ml blood). The serum is then analyzed for glucose and insulin using standard methodologies.

The assay uses a formulated and calibrated peptide stock in PBS. Normally, this stock is a prediluted 100 μM stock. However, a more concentrated stock with approximately 1 mg agonist per mL is used. The specific concentration is always known.

TABLE 7

| | Dose | | Effects on | Effects on | |
|---|---|---|---|---|---|
| Peptide | (μg/kg) | Route | insulin | glucose | Exposure |
| P31 | 10 | iv | +91% AUC_{(0-10 min)} | None | Not |
| (2212924) | | | | | measured |

Oral Glucose Tolerance Test (OGTT):

The effect of a selective VPAC2 receptor peptide agonist (P31) on plasma insulin and glucose is evaluated during OGTT in conscious Wistar rats. The maximal dose of agonist is 10 μg/kg. Since the peptide is given intravenously and has a very short half-life, a delay between glucose and compound administrations is applied.

Protocol details are as follows:

| SPECIES/STRAIN/ | Rat/Wistar Unilever/approximately 275-300 g | | | | |
|---|---|---|---|---|---|
| WEIGHT | | | | | |
| TREATMENT | Single dose | | | | |
| DURATION | | | | | |
| GROUP/COMPOUND/ | | | Dose | Injection Time | |
| DOSE/NUMBER/ | Group | Compound | (μg/kg) | (min) | Number/Sex |
| SEX | 1 | vehicle | 0 | 15 | 6M |
| | 2 | vehicle | 0 | 30 | 6M |
| | 5 | P31 | 10 | 15 | 6M |
| | 6 | P31 | 10 | 30 | 6M |

| | |
|---|---|
| DOSE | 1.5 mL/kg/iv |
| VOLUME/ROUTE | |
| VEHICLE | 8% PEG300, 0.1% BSA in water. |
| FOOD/WATER | Fasted overnight prior to the test. |
| REGIMEN | |
| LIVE-PHASE | Animals will be trained for contention, gavage, and tail |
| PARAMETERS | massage 2 days before the experiment. Animals will be |
| | sacrificed at the end of the test. Two animals of each group |
| | are tested on each day. |
| OGTT: Performed on | Glucose orally: 2.5 g/kg as 50% solution (5 mL/kg) at time = 0. |
| conscious, non- | Compound IV: 15 or 30 min after glucose. |
| cannulated rats of each | Blood samplings: (300 μL from tail tip; EDTA as |
| group. | anticoagulant; aprotinin and PMSF as antiproteolytics; kept on |
| | ice): Groups 1, 3, and 5: before glucose (time 0), at 15 min |
| | (just before compound), and at 20, 30, 45, 75, 105, and 135 |
| | minutes. |
| | Groups 2, 4, and 6: before glucose (time 0), at 30 min (just |
| | before compound), and at 35, 45, 60, 90, 120, and 150 |
| | minutes. |
| | Parameters determined: Insulin, glucose |
| TOXICOKINETICS | Plasma samples remaining after insulin measurements will be |
| | kept at −20° C. and sent to Hamburg for determination of |
| | compound levels. |

Results are as follows:

TABLE 8

| | | Dose | | Effects on | |
|---|---|---|---|---|---|
| Study | Peptide | (μg/kg) | Route | insulin | Effects on glucose |
| OGTT (15 min), | P31 | 10 | iv | +71% AUC_{(0-75 min)} | −22% AUC_{(0-135 min)} |
| #1 | (2212924) | | | | |
| OGTT (15 min), | P31 | 0.5 | iv | +16% AUC_{(0-75 min)} | −5% AUC_{(0-135 min)} |
| #2 | (2212924) | | | | |
| OGTT (15 min), | P31 | 10 | iv | +17% AUC_{(0-75 min)} | −2% AUC_{(0-135 min)} |
| #2 | (2212924) | | | | |
| OGTT (30 min) | P31 | 10 | iv | +85% AUC_{(0-90 min)} | −1% AUC_{(0-150 min)} |
| | (2212924) | | | | (NS) |

### EXAMPLE 6

### Rat Serum Stability Studies

In order to determine the stability of VPAC2 receptor peptide agonists in rat serum, obtain CHO-VPAC2 cells clone #6 (96 well plates/50,000 cells/well and 1 day culture), PBS 1× (Gibco), the peptides for the analysis in a 100 μM stock solution, rat serum from a sacrificed normal Wistar rat, aprotinin, and a DiscoveRx assay kit. The rat serum is stored at 4° C. until use and is used within two weeks.

On Day 0, prepare two 100 μL aliquots of 10 μM peptide in rat serum by adding 10 μL peptide stock to 90 μL rat serum for each aliquot. Add 250 kIU aprotinin/mL to one of these aliquots. Store the aliquot with aprotinin at 4° C. Store the aliquot without aprotinin at 37° C. Allow the aliquots to incubate for 18 hours.

On Day 1, after incubation of the aliquots prepared on day 0 for 18 hours, prepare an incubation buffer containing PBS+1.3 mM CaCl₂, 1.2 mM MgCl₂, 2 mM glucose, and 0.25 mM IBMX. Prepare a plate with 11 serial 5× dilutions of peptide for the 4° C. and 37° C. aliquot for each peptide studied. Use 2000 nM as the maximal concentration if the peptide has an EC₅₀ above 1 nM and 1000 nM as maximal concentration if the peptide has an EC₅₀ below 1 nM from the primary screen (see Example 3). Wash the plate(s) with cells twice in incubation buffer. Allow the plates to hold 50 μL incubation media per well for 15 minutes. Transfer 50 μL solution per well to the cells from the plate prepared with 11 serial 5× dilutions of peptide for the 4° C. and 37° C. aliquot for each peptide studied, using the maximal concentrations that are indicated by the primary screen, in duplicate. This step dilutes the peptide concentration by a factor of two. Incubate in room temperature for 30 minutes. Remove the supernatant. Add 40 μL/well of the DiscoveRx antibody/extraction buffer. Incubate on the shaker (300 rpm) for 1 hour. Proceed as normal with the DiscoveRx kit. Include cAMP standards in column 12. Determine EC₅₀ values from the cAMP assay data. The remaining amount of active peptide is estimated by the formula EC_{50, 4C}/EC_{50, 37C} for each condition.

TABLE 9

| | Rat Serum Stability |
|---|---|
| | (estimated purity in % |
| Peptide | after 18 hours) |
| P104 | 1.01 |
| P106 | 0.11 |
| P119 | 19 |
| P119 | 37.23 |
| P120 | 5 |
| P133 | <2.00 |
| P138 | 0.48 |
| P139 | 0.33 |
| P142 | 0.51 |
| P146 | 0.53 |
| P147 | 0.07 |
| P148 | <0.10 |
| P149 | 0.29 |
| P150 | 0.21 |
| P151 | 0.35 |
| P152 | 0.06 |
| P153 | <0.16 |
| P155 | 0.25 |
| P158 | 0.32 |
| P159 | 0.15 |
| P160 | <4.25 |
| P163 | <1.25 |
| P165 | 7.28 |
| P167 | 14.98 |
| P168 | 10.47 |
| P169 | 0.40 |
| P170 | 0.31 |
| P171 | 1.78 |
| P172 | 5.95 |
| P173 | 1.04 |
| P174 | 1.91 |
| P175 | <0.11 |
| P176 | <0.14 |
| P177 | 0.49 |
| P179 | 1.66 |
| P180 | 1.14 |
| P181 | <0.53 |
| P182 | 0.15 |
| P183 | 0.18 |
| P184 | 0.29 |
| P185 | 0.27 |
| P186 | 0.25 |
| P193 | 0.25 |
| P194 | 1.11 |
| P197 | 5.68 |
| P203 | <0.09 |
| P205 | 35.02 |
| P207 | <0.29 |
| P209 | 0.13 |
| P210 | <0.91 |
| P212 | 0.21 |
| P213 | 0.04 |
| P214 | 0.07 |
| P215 | 0.08 |
| P216 | 0.06 |
| P220 | 0.08 |
| P221 | 0.87 |
| P222 | 0.23 |
| P223 | 1.52 |
| P228 | <5.00 |
| P229 | <5.00 |
| P230 | 0.53 |
| P231 | 0.57 |
| P232 | <5.00 |
| P233 | 1.27 |
| P234 | 0.69 |
| P235 | 2.62 |
| P236 | 0.79 |
| P240 | 43.87 |
| P241 | 62.55 |
| P242 | 40.28 |
| P243 | 12.12 |
| P244 | 30.64 |
| P249 | 12.9 |
| P250 | 7.11 |
| P251 | 6.63 |
| P252 | 1.19 |
| P253 | 1.27 |
| P258 | 9.47 |
| P259 | 17.65 |
| P260 | 28.57 |
| P261 | 27.06 |
| P262 | 10.14 |
| P263 | 11.72 |
| P264 | 19.22 |
| P265 | 8.70 |
| P269 | 23.85 |
| P284 | 27.89 |
| P289 | 0.04 |
| P290 | 0.11 |
| P291 | 28.09 |
| P292 | 33.43 |
| P293 | 32.70 |
| P294 | 24.25 |
| P295 | 22.22 |
| P296 | 42.26 |
| P297 | 21.17 |
| P298 | 30.97 |
| P301 | 6.86 |
| P302 | 21.43 |
| P305 | 0.23 |
| P307 | 0.32 |
| P308 | 0.97 |
| P31 | <1 |
| P31 | 0.88 |
| P314 | 8.44 |
| P315 | 24.00 |
| P316 | 15.06 |
| P317 | 33.34 |
| P318 | 29.95 |
| P319 | 13.53 |
| P320 | 28.59 |
| P322 | 42.18 |
| P323 | 16.81 |
| P324 | 13.64 |
| P325 | 13.13 |
| P326 | 90.10 |
| P329 | 47.98 |
| P333 | 40.90 |
| P335 | 98.66 |
| P338 | 36.61 |
| P341 | 12.82 |
| P342 | 25.96 |
| P344 | 50.19 |
| P345 | 20.23 |
| P346 | 43.44 |
| P349 | 41.22 |
| P350 | 25.83 |
| P351 | 18.52 |
| P352 | 45.09 |
| P353 | 31.23 |
| P354 | 15.10 |
| P355 | 35.94 |
| P356 | 45.17 |
| P364 | 86.23 |
| P365 | 91.13 |
| P366 | 92.00 |
| P99 | 0.61 |

TABLE 10

| | Rat Serum Stability |
|---|---|
| Peptide | (Estimated purity in % after 72 hours) |
| P89 | <0.4 |
| P240 | 10.4 |
| P242 | 3.8 |
| P244 | 6.2 |
| P264 | 0.9 |
| P322 | 1.3 |
| P326 | 49.6 |
| P329 | 11.2 |
| P333 | 61.2 |
| P335 | 22.0 |
| P344 | 43.0 |
| P346 | 27.6 |
| P352 | 2.2 |
| P317 | 21.5 |

### EXAMPLE 7

### Pharmacokinetic Assay

An analysis of active peptide levels in rat plasma is conducted after IV injection of 10 μg/kg of each peptide. An IVGTT with glucose is given immediately after T=0 to 6 animals per condition. Samples are taken at 0, 2, 4, 6, and 10 minutes after injection.

For cell handling, Plate 50,000 cells/well and keep in culture over night. Wash cell twice in PBS and add 50 μl/well stimulation medium consisting of PBS+1.2 MgCl2, 1.3 CaCl2, 2 glucose and 0.5 IBMX. Incubate 15 minutes and add 50 μl/well of the plasma samples (see layout below). Incubate 30 minutes, remove the supernatant and proceed as normal with the DiscoveRx assay. Prepare plates in duplicate. Protease and peptidase inhibitor are present in all plasma samples.

TABLE 11

| | Exposure | | Exposure (10 μg/kg; | | |
|---|---|---|---|---|---|
| | (10 μg/kg | Exposure | clearance | Exposure (10 μg/kg, | Exposure |
| | i.v., Cmax; | (10 μg/kg; | in | Vdist in | (AUC last, |
| Peptide | nM) | t½, min) | μg/(min * nM * kg) | μg/(nM * kg) | nM * min) |
| P31 | 4.37 | 12 | 0.65 | 10 | 18.5 |
| P89 | 5.45 | 1.32 | 0.33 | 0.64 | 31.41 |
| P104 | 3.19 | 2.35 | 0.87 | 2.64 | 21.3 |
| P264 | 0.96 | 1.3 | 2.8 | 5.1 | 3.6 |
| P261 | 12.1 | 3.2 | 0.18 | 0.8 | 53.9 |
| P292 | 5.37 | 5.5 | 0.2 | 1.9 | 41.3 |

TABLE 12

| | Exposure | | Exposure (0.5 μg/kg; | | |
|---|---|---|---|---|---|
| | (0.5 μg/kg | Exposure | clearance | Exposure (0.5 μg/kg, | Exposure |
| | i.v., Cmax; | (0.5 μg/kg; | in | Vdist in | (AUC last, |
| Peptide | nM) | t½, min) | μg/(min * nM * kg) | μg/(nM * kg) | nM * min) |
| P292 | 0.12 | 15.0 | 0.4 | 9.5 | 1.0 |

### EXAMPLE 8

### DPP-IV HPLC Assays

Part 1: Formulation of Selective VPAC2 Receptor Peptide Agonists:

Approximately 2 mg of lyophilized peptide is weighed and dissolved in approximately 1.6 mL de-ionized water. If the peptide does not dissolve, the pH is adjusted with 1M NaOH to between pH 10.0 and 10.5. After incubation at room temperature for 30 minutes, 1/10^{th} of the original volume 10×PBS is added. The pH is adjusted to between pH 7.2 and 7.6. The peptide solution is filtered through a 0.22 μm Millex-GV syringe filter (Millipore, Bedford Mass., USA). The peptide concentration is determined through absorption at 280 nm. The peptide concentration is then adjusted to 100 μM. The peptides are frozen at −20° C. for further use.

Part 2: In Vitro Incubation of Selective VPAC2 Receptor Peptide Agonists with Purified Dipeptidyl-Peptidase IV (DPP-IV):

The stability of selective VPAC2 receptor peptide agonists against proteolysis by DPP-IV is determined using 100 μL of a 100 μM peptide solution in 1×PBS. A 10 μL solution is removed and quenched with 40 μL of 0.1% trifluoroacetic acid (TFA)/20% acetonitrile (ACN). This solution (20 μL) is analyzed by reversed-phase HPLC. The reversed-phase analysis consists of a Zorbax 300SB-C8 column (3.5 micron, 4.6×50 mm, Alltech Associates, Inc., Deerfield Ill., USA) running a 15-40% B gradient over 15 minutes at 60° C. where A-buffer is 0.1% (v/v) TFA in water and B-buffer is 0.085% (v/v) TFA in ACN. The peak area is integrated. This peak area serves as an internal control as 100% intact peptide.

A 10 μL aliquot of a 1.12 mU/μL solution of DPP-IV (Sigma, St. Louis, La., USA) is added to 90 μL of a 100 μM solution of peptide, resulting in a substrate concentration of 90 μM peptide. The reaction mixture is then stored at 37° C. At various time-points, 10 μL of solution is removed, quenched with 40 μL 0.1% TFA/20% ACN, and analyzed by reversed-phase HPLC as described above. The remaining full length peptide concentration (nM) at each timepoint, except time=0, is calculated using following formula:

$$\frac{peak area [ time x ] * concentration [ t 0 ]}{peak area [ time 0 ] * 0.9}$$

For the time=0 timepoint, the concentration (nM) is calculated using the following formula:

$$\frac{peak area [ time x ] * initial substrate concentration [ 9 nM ]}{peak area [ time 0 ]}$$

TABLE 13
Concentration (nM) of Remaining Main Peak by RP-HPLC after
Incubation of Peptide with Purified Porcine DPP-IV

| Study | | | | | Time¹ = |
|---|---|---|---|---|---|
| Number | Sample | Time¹ = 0 | Time¹ = 2 | Time¹ = 6 | 24 |
| 1 | VPAC2-P31 | 9.0 | 6.7 | 5.6 | 3.9 |
| 1 | VPAC2-P44 | 9.0 | 6.5 | 3.5 | 0.8 |
| 1 | VPAC2-P47 | 9.0 | 7.9 | 6.1 | 2.8 |
| 2 | VPAC2-P44, 1 | 9.0 | 6.4 | 5.5 | 1.5 |
| 2 | VPAC2-P44, 2 | 9.0 | 7.9 | 4.8 | 1.4 |
| 3 | VPAC2-P31 | 9.0 | 8.0 | 6.7 | 2.2 |
| 4 | VPAC2-P31 | 9.0 | 7.0 | 6.1 | 2.6 |
| 4 | VPAC2-P44 | 9.0 | 4.5 | 2.3 | 0.4 |
| 4 | VPAC2-P104 | 9.0 | 7.8 | 6.6 | 4.1 |
| 4 | VPAC2-P119 | 9.0 | 8.0 | 6.6 | 4.2 |

¹Time = Hours at 37° C.

Other modifications of the present invention will be apparent to those skilled in the art without departing from the scope of the invention.

1-36. (canceled)

37. A VPAC2 receptor peptide agonist, comprising the amino acid sequence:
(SEQ ID NO: 20)
Xaa₁-Xaa₂-Xaa₃-Xaa₄-Xaa₅-Xaa₆-Thr-Xaa₈-Xaa₉-Xaa₁₀-
Thr-Xaa₁₂-Xaa₁₃-Xaa₁₄-Xaa₁₅-Xaa₁₆-Xaa₁₇-Ala-Xaa₁₉-
Xaa₂₀-Xaa₂₁-Xaa₂₂-Xaa₂₃-Xaa₂₄-Xaa₂₅-Xaa₂₆-Xaa₂₇-
Xaa₂₈-Xaa₂₉-Xaa₃₀-Xaa₃₁-Xaa₃₂
wherein:
Xaa₁ is: His, dH, or is absent;
Xaa₂ is: dA, Ser, Val, Gly, Thr, Leu, dS, Pro, or Aib;
Xaa₃ is: Asp or Glu;
Xaa₄ is: Ala, Ile, Tyr, Phe, Val, Thr, Leu, Trp, Gly, dA, Aib, or NMeA;
Xaa₅ is: Val, Leu, Phe, Ile, Thr, Trp, Tyr, dV, Aib, or NMeV;
Xaa₆ is: Phe, Ile, Leu, Thr, Val, Trp, or Tyr;
Xaa₈ is: Asp, Glu, Ala, Lys, Leu, Arg, or Tyr;
Xaa₉ is: Asn, Gln, or Glu;
Xaa₁₀ is: Tyr, Trp, or Tyr(OMe);
Xaa₁₂ is: Arg, Lys, hR, Orn, Aib, Cit, or Ala;
Xaa₁₃ is: Leu, Phe, Glu, Ala, or Aib;
Xaa₁₄ is: Arg, Leu, Lys, Ala, hR, Orn, Phe, Gln, Aib, or Cit;
Xaa₁₅ is: Lys, Ala, Arg, Glu, Leu, hR, Orn, Phe, Gln, Aib, K(Ac), or Cit;
Xaa₁₆ is: Gln, Lys, Ala, hR, Orn, or Cit;
Xaa₁₇ is: Val, Ala, Leu, Ile, Met, Nle, Lys, or Aib;
Xaa₁₉ is: Ala, Gly, or Leu;
Xaa₂₀ is: Lys, Gln, hR, Arg, Ser, Orn, Ala, Aib, Trp, Thr, Leu, Ile, Phe, Tyr, Val, K(Ac), or Cit;
Xaa₂₁ is: Lys, Arg, Ala, Phe, Aib, Leu, Gln, Orn, hR, K(Ac) or Cit;
Xaa₂₂ is: Tyr, Trp, Phe, Thr, Leu, Ile, Val, Tyr(OMe), Ala, or Aib;
Xaa₂₃ is: Leu, Phe, Ile, Ala, Trp, Thr, Val, or Aib;
Xaa₂₄ is: Gln, or Asn;
Xaa₂₅ is: Ser, Asp, Phe, Ile, Leu, Thr, Val, Trp, Gln, Asn, Tyr, Aib, or Glu;
Xaa₂₆ is: Ile, Leu, Thr, Val, Trp, Tyr, Phe or Aib;
Xaa₂₇ is: Lys, hR, Arg, Gln, Orn, or dK;
Xaa₂₈ is: Asn, Gln, Lys, Arg, Aib, Orn, hR, Cit, Pro, or dK;
Xaa₂₉ is: Lys, Ser, Arg, Asn, hR, Orn, Cit, Aib or is absent;
Xaa₃₀ is: Arg, Lys, Ile, hR, Cit, Aib, Orn, or is absent;
Xaa₃₁ is: Tyr, His, Phe, or is absent; and
Xaa₃₂ is: Cys, or is absent;
provided that if Xaa₂₉, Xaa₃₀, or Xaa₃₁ is absent, the next amino acid present downstream is the next amino acid in SEQ ID NO: 20,
and a C-terminal extension wherein the N-terminus of said C-terminal extension is linked to the C-terminus of said peptide of SEQ ID NO: 20, and wherein said C-terminal extension is selected from the group consisting of GGPSSGAPPPS (SEQ ID NO: 10), GGPSSGAPPPS-NH₂ (SEQ ID NO: 11), GGPSSGAPPPC (SEQ ID NO: 22), GGPSSGAPPPC-NH₂, (SEQ ID NO: 23), GRPSSGAPPPS (SEQ ID NO: 24), and GRPSSGAPPPS-NH₂ (SEQ ID NO: 25), or a pharmaceutically acceptable salt thereof.

38-40. (canceled)

41. The VPAC2 receptor peptide agonist according to claim 37, further comprising an N-terminal modification, wherein said N-terminal modification is the addition of a group selected from the group consisting of acetyl, propionyl, butyryl, pentanoyl, hexanoyl, methionine, methionine sulfoxide, 3-phenylpropionyl, phenylacetyl, benzoyl, norleucine, D-histidine, isoleucine, and 3-mercaptopropionyl.

42. The VPAC2 receptor peptide agonist according to claim 41, wherein said N-terminal modification is the addition of acetyl or hexanoyl.

43. The VPAC2 receptor peptide agonist according to claim 37, comprising the amino acid sequence:
(SEQ ID NO: 21)
His-Xaa₂-Xaa₃-Xaa₄-Xaa₅-Phe-Thr-Xaa₈-Xaa₉-Tyr-Thr-
Xaa₁₂-Leu-Xaa₁₄-Xaa₁₅-Xaa₁₆-Xaa₁₇-Ala-Xaa₁₉-Xaa₂₀-
Xaa₂₁-Xaa₂₂-Leu-Xaa₂₄-Xaa₂₅-Xaa₂₆-Xaa₂₇-Xaa₂₈-
Xaa₂₉-Xaa₃₀-Xaa₃₁
wherein:
Xaa₂ is: dA, Ser, Val, dS, or Aib;
Xaa₃ is: Asp or Glu;
Xaa₄ is: Ala, dA, or Aib;
Xaa₅ is: Val, Leu, dV, or Aib;
Xaa₈ is: Asp, Glu, or Ala;
Xaa₉ is: Asn, Gln, or Glu;
Xaa₁₂ is: Ala, Arg, Lys, hR, or Orn;
Xaa₁₄ is: Arg, Leu, Lys, Ala, hR, Orn, Phe, Gln, Aib, or Cit;
Xaa₁₅ is: Lys, Ala, Arg, Leu, Orn, Phe, Gln, Aib, or K(Ac);
Xaa₁₆ is: Gln, or Lys;
Xaa₁₇ is: Val, Ala, Leu, Ile, Met, Nle, or Lys;
Xaa₁₉ is: Ala, or Leu;
Xaa₂₀ is: Lys, Gln, hR, Arg, Ser, Ala, Aib, Trp, Thr, Leu, Ile, Phe, Tyr, Val, or K(Ac);
Xaa₂₁ is: Lys, Arg, Ala, Phe, Aib, Leu, Gln, K(Ac), or Orn;
Xaa₂₂ is: Tyr, Trp, Phe, Leu, Ile, or Val;
Xaa₂₄ is: Gln, or Asn;
Xaa₂₅ is: Ser, Phe, Ile, Leu, Thr, Val, Trp, Gln, Asn, Tyr, or Aib;
Xaa₂₆ is: Ile, Leu, Thr, Val, Trp, Tyr, Phe or Aib;
Xaa₂₇ is: Lys, hR, Arg, dK, or Orn;
Xaa₂₈ is: Asn, Gln, Lys, hR, Aib, Orn, dK, or Pro;
Xaa₂₉ is: Lys, Ser, Arg, hR, Orn, or is absent;
Xaa₃₀ is: Arg, Lys, or is absent; and
Xaa₃₁ is: Tyr, Phe, or is absent;
provided that if Xaa₂₉, or Xaa₃₀ is absent, the next amino acid present downstream is the next amino acid in SEQ ID NO: 21,
and a C-terminal extension wherein the N-terminus of said C-terminal extension is linked to the C-terminus of said peptide of SEQ ID NO: 21, and wherein said C-terminal extension is selected from the group consisting of GGPSSGAPPPS (SEQ ID NO: 10), GGPSSGAPPPS-NH₂ (SEQ ID NO: 11), GGPSSGAPPPC (SEQ ID NO: 22), GGPSSGAPPPC-NH₂, (SEQ ID NO: 23), GRPSSGAPPPS (SEQ ID NO: 24), and GRPSSGAPPPS-NH₂ (SEQ ID NO: 25), or a pharmaceutically acceptable salt thereof.

44-46. (canceled)

47. The VPAC2 receptor peptide agonist according to claim 43, further comprising an N-terminal modification, wherein said N-terminal modification is the addition of a group selected from the group consisting of acetyl, propionyl, butyryl, pentanoyl, hexanoyl, methionine, methionine sulfoxide, 3-phenylpropionyl, phenylacetyl, benzoyl, norleucine, D-histidine, isoleucine, and 3-mercaptopropionyl.

48. The VPAC2 receptor peptide agonist according to claim 47, wherein said N-terminal modification is the addition of acetyl or hexanoyl.

49. A method of treating non-insulin-dependent diabetes or insulin-dependent diabetes in a patient in need thereof, comprising administering to said patient a VPAC2 receptor peptide agonist according to claim 37.

50-51. (canceled)

52. The VPAC2 receptor peptide agonist according to claim 37, comprising the amino acid sequence:
(SEQ ID NO: 285)
Hexanoyl-HSDAVFTDNYTOrnLRAibQLAAAibKYLQSIOrnNOrnGG
PSSGAPPPS.
